(19) United States
(12) Patent Application Publication
(10) Pub. No.: US 2024/0390446 A1
(43) Pub. Date: Nov. 28, 2024

(54) COMPOSITIONS AND METHODS FOR TREATING OR PREVENTING INFLAMMATORY DISEASES INCLUDING DIABETES AND THYROID DISEASES

(71) Applicant: Rachel ALKALAY, Tel Aviv (IL)

(72) Inventor: Rachel ALKALAY, Tel Aviv (IL)

(21) Appl. No.: 18/694,656

(22) PCT Filed: Sep. 22, 2022
(86) PCT No.: PCT/IB2022/058945

§ 371 Date: Mar. 22, 2024

Related U.S. Application Data
(60) Provisional application No. 63/247,328, filed on Sep. 23, 2021.

(51) Int. Cl. A61K 36/71; A61K 36/185; A61K 36/22; A61K 36/258; A61K 36/324; A61K 36/424; A61K 36/53; A61K 36/534; A61K 36/537; A61K 36/54; A61K 36/738; A61K 36/82; A61K 36/85; A61K 36/899; A61K 36/9064; A61K 45/06; A61P 3/10

(52) U.S. Cl. CPC A61K 36/71; A61K 36/185; A61K 36/22; A61K 36/258; A61K 36/324; A61K 36/424; A61K 36/53; A61K 36/534; A61K 36/537; A61K 36/54; A61K 36/738; A61K 36/82; A61K 36/85; A61K 36/899; A61K 36/9064; A61K 45/06; A61P 3/10

(57) ABSTRACT

A method of preventing or treating an inflammatory disease and/or conditions associated with inflammation and/or biomarkers associated with inflammation in a subject in need thereof is provided. Also provided are pharmaceutical compositions, articles and foods for the treatment of an inflammatory disease using natural active compounds such as herb and flower extracts.

### FIELD AND BACKGROUND OF THE INVENTION

The present invention, in some embodiments thereof, relates to compositions and methods for treating or preventing inflammatory diseases.

Inflammation involves the activation of the immune system in response to harmful stimuli, such as, e.g., a pathogen, infection, irritant, or damage to cells. As a stereotyped response, inflammation is a mechanism of innate immunity, as compared to adaptive immunity, which is specific for each pathogen. Inflammation can be classified as either acute or chronic. Generally speaking, acute inflammation is mediated by granulocytes, while chronic inflammation is mediated by mononuclear cells such as monocytes and lymphocytes.

Acute inflammation is an initial protective response of the body to remove an injurious stimulus by maintaining tissue integrity and contributing to tissue repair. It is a part of the body's natural defense system against injury and disease, and in the absence of acute inflammation, wounds and infections would never heal and progressive destruction of the tissue would compromise the survival of the organism.

The process of acute inflammation is initiated by cells already present in all tissues, mainly resident macrophages, dendritic cells, histiocytes, Kupffer cells, mastocytes, vascular endothelial cells, and vascular smooth muscle cells. At the onset of a harmful stimulus, these cells undergo activation and release inflammatory mediating and sensitizing molecules, such as, e.g., pro-inflammatory cytokines, pro-inflammatory prostaglandins, leukotrienes, histamine, serotonin, neutral proteases, bradykinin and nitric oxide. These inflammatory molecules modulate a complex series of biological events involving cellular and acellular components of the local vascular system, the immune system, and the injured tissue site to propagate and mature the inflammatory response. These events are responsible for eliciting an acute inflammatory response, typically characterized by 1) vasodilatation, which increases blood flow into the tissue thereby causing erythema (redness and warmth), which may extend beyond this site (the flare response); 2) blood vessel permeability, which increases plasma leakage into the tissue thereby causing edema (swelling); 3) altering the excitability of certain sensory neurons causing hypersensitivity and pain; 4) stimulating the release of inflammation inducing molecules, such as, e.g., neuropeptides like substance P (SP) and calcitonin gene-related peptide (CGRP), prostaglandins, and amino acids like glutamate, from the peripheral nerve endings; and 5) increasing migration of leukocytes, mainly granulocytes, from the blood vessels into the tissue. An acute inflammatory response requires constant stimulation to be sustained and must be actively terminated when no longer needed. Hence, acute inflammation ceases once the injurious stimulus has been removed.

However, severe or prolonged noxious stimulation results in a chronic inflammatory response that leads to a progressive shift in the type of cells present at the site of tissue injury. Chronic inflammation may be characterized as the simultaneous destruction and healing of tissue from the inflammatory process, with the net result of provoking injury rather than mediating repair. As such, chronic inflammation is a disease. As an inflammatory response can occur anywhere in the body, chronic inflammation has been implicated in the pathophysiology of a wide range of seemingly unrelated disorders which underlay a large and varied group of human diseases. For example, chronic inflammation is involved in diseases as diverse as cardiovascular diseases, cancers, allergies, obesity, diabetes, digestive system diseases, liver diease, degenerative diseases, auto-immune disorders, anemia, sickle cell anemia, fibromyalgia, and Alzheimer's disease or other related dementia e.g., vascular dementia, mixed dementia, fronto-temporal dementia, Lewy-body dementia, memory loss, cholesterol disorders, hair loss, depression, hormonal disorders. PMS (premenstrual Syndrome), Menopause Symptoms, PCOS (Polycystic ovary syndrome), sleep disruptions/disorders, headaches, loss of weight, overweight, vertigo, dizziness, lack of focus, erectile disfucntion, fatigue and weakness, muscle pain, diarrhea, loss of smell, loss of taste, skin rash, back pain, chest pain, coughing, runny nose, sore throat, hoarsness, nausea, vomiting, fever, eye infection, ear infection, Schizophrenia, delusions, hallucinations, paranoia, disorganized speech, Autism, PTSD, Aspergers, ADD, ADHD, depression, anxiety, delliurm, glaucoma, fibrotic formation/function in the cornea, reactive attachment disorder, intellectual disability, Neurodegenerative diseases, includingAmyotrophic lateral sclerosis, (ALS, Lou Gehrig's disease), Charco-Marie-Tooth disease, Chronic traumatic encephalopathy, cystic fibrosis, cytochrome c oexidase, Ehlers-Danlos syndrome, Fibrodysplais ossificans progressive, Friedreich's ataxis, Frontotemporal deminatia, Huntington disease, infantile neuroaxonal dystrophy, keratoconus, Leukodystrophies, maculardegeneration, marfans syndrome, mitochondrial myppathies and mitochondrial DNA depletion syndrome, multiple sclerosis, multiple system atrophy, muscular dystrophies, neuronal ceroid lipfuscinosis, Niemann-Pick diseases, osteoarthritis, osteoporosis, pulmonary arterial hypertension, all prion diseases, progressive supranuclear palsy, retinitis pigmentosa, rheumatoid arthritis, Sandoff diseases, spinal muscular atrophy, substance use disorder, subacute sclerosing, Tay-Sachs disease,

Parkinson disease, essential tremor, Parkinsonism, NPH (Normal pressure hydrocelphalus).

Chronic inflammation is also associated with aging disorders, including disrupted communication between cells, genome mutations, telomere shortening, hanges in DNA's chemical structure, degradation of cellular proteins, impaired mitochondrial functioning, and nonrenewal of stem cells progeria. Aging and skin damage include, acne skin wounds, skin scarring, loosening of the skin and darker pigmentation spots forming on the skin.

Attempts to treat chronic inflammation have met with limited success. This is due, in part, to the fact that the etiology of chronic inflammation is a complex response based in part on the various inflammation inducing molecules and the multitude of inflammation mediating and sensitizing molecules that appear to elicit inflammation via redundant mechanisms. In addition, besides blocking pro-inflammatory molecules, many anti-inflammatory drugs, also inhibit regulatory loops that release endogenous anti-inflammatory molecules. For example, NSAIDs reduce inflammation by blocking the enzymatic activity of cyclooxygenase, a key enzyme that catalyzes the conversion of arachidonic acid to prostaglandins and leukotrienes. Thus, NSAIDs reduce inflammation by preventing the synthesis of all prostaglandins. However, NSAIDs not only prevent the synthesis of proinflammatory prostaglandins, these compounds also prevent the synthesis of anti-inflammatory prostaglandins. Hence, NSAIDs have limited success as they block endogenous anti-inflammatory response, which in some instances may prolong chronic inflammation. Therefore, compounds, compositions, uses, and methods preferentially inhibiting pro-inflammatory responses would be highly desirable for the treatment of inflammation.

Type I diabetes mellitus (T1DM) is a multi-factorial autoimmune disease characterized by an immune-mediated destruction of pancreatic B cells. Studies have shown that environmental factors contribute to the constant rise off TIDM all over the world. Viral infections represent one of the environmental risks and epidemiological data show that TIDM incidence increases after epidemics due to enteroviruses, and that enteroviral RNA can be detected in the blood of >50% of T1DM patients at the time of disease onset (See Galleri et al, 2012).

Studies have shown that chronic HCV infection is associated with an increased risk of developing insulin resistance (IR) and type 2 diabetes (T2D). Clinical and experimental data suggest that HCV contributes to the pathogenesis of T2D (Negro et. al, 2009). Patients with chronic HCV infection have an increased prevalence of type 2 diabetes, and this prevalence is independent of cirrhosis (see Knobler et. al 2000). 20) It is clear that there is a long felt and unmet need to provide effective compositions for inflammatory diseases, including diabetes I, II and gestational diabetes.

The thyroid is a gland of the endocrinic system. It is located at the front of the neck consisting of two lobes, connected by a thin band called the thyroid isthmus. The thyroid secretes three hormones: triiodothyronine (T3), thyroxine (T4) and calcitonin. T3 and T4 influence the metabolic rate, protein synthesis, and in children, growth and development. Calcitonin plays a role in calcium homeostasis. Apart from thyroid types of cancer, there are several specific diseases of the thyroid, that may result in either Hyperthyroidism or Hypothroidism according to the effect on the thyroid. Hyperthyroidismrelated dieases inclulde Hashimoto's disease, Grave's disease, Goieter disease, Thyroid Nodules disease. Hypothyroidism mainly related to Hashimoto's disease.

US 2008/0038367 describes nutritional supplements containing extracts of naturally occurring sources that contain nutrients that show biological and therapeutic activity. Particularly, such supplements are effective in treating viral, bacterial and microbial infections, as well as inflamation. In particular, extracts of these herbs and flowers such as Black Cumin (Nigella sativa) and Sumac (Rhus coriara) are provided in the range of about 0.5 g to 60 g per liter. Tryptophan may also be added to such extracts.

WO 2020/044123 describes cannabinoid furmulations, particularly in combination with Boswellia serrata extract, for treating conditions and disorders implicated by the physiological functions of what are believed to be cannabinoid receptors. One such function is inflammation, for which these formulations are found to be effective, when accompanied by diabetes. Extracts of Boswellia serrata are provided with cannbinoids in oral formulations in amounts ranging between 100 to 1000 mg. Particularly, the active compound in the Boswellia serrata is 3-O-actetyl-1 1-ketobeta-boswellic acid (AKBA). Cannabinoid compounds are administered in doses between 0.1 to 750 mg, but their daily amounts vary per the treated individual and the synergistic effect in combination with other health products obtained from other herbs such as Siberian Ginseng (Eleuthero root) and Thyme (Thymus vulgaris).

US 2012/022053 describes formulations that contain theaflavin (TF) compositions and glucosamine compositions for treating conditions associated with chronic inflammation that reslt from chronic viral or bacterial infections. The TF compositions are obtained from extracts of Black tea, and polyphenols of fermented TF found in green tea. Natural oils may be added to these compositions, including those that contain a high concentration of phenols, for example origanum, thyme and rosemary oils. Vehicles of the active agents include naturally obtained seasame oil.

US 2011/00586052 describes formulations for treating innate immunity signaling that with ginseng fractions made from Panax quinquefolius. These formulations are suitable for treating viral infections from a respiratory or mucusally transmitted virus, such as influenza and corona virus.

US 2010/0119558 describes pharmaceutical compositions that contain NGNA (n-glycolylneuraminic acid), which is a naturaceutical, namely bioactive, compound that is found in sea cucumber. Other biocactive compounds can be found in various natural sources including Siberian Ginseng. Such compositions are effective in treating viral infections such as influenza, rhinoviruses, adenoviruses, herpes viruses and noroviruses.

EP 2 482 831 describes compositions from extracts of Cridothymus capitatus, Origanum didtamnus and Slavia fruticosa or Salvia pomifera. Such compositions are found to be effective in treating viruses that adversely affect the respiratory system in a human. Preferably, these ingredients are present in the composition in corresponding ratios of 40-80%, 3-15% and 17-45%.

The pharmaceutical compositions and food additives in these publications are essentially combinations of particular herbal extracts that comprise particular active compounds or combinations of naturally occurring active compounds for treating certain medical disorders and conditions. Additionally, particular relative and/or absolute amounts of such active compounds or extracts are used in these combinations to specifically target such conditions and disorders, especially viral or bacterial effected inflammation. Further, these compositions and extracts are produced in particular methods to obtain such effective amounts of the active compounds to treat these disorders and conditions. Therefore, to obtain any pharmaceutical effect from herb extracts, their active compounds and combinations, particular relative and/or absolute amounts of selected combinations of extract sources should be obtained in appropriate methods. In addition, effective treatment should be shown to prove equivalence or at least resemblance of such combinations to any other combination with similar or even same ingredients.

None of these publications describes or suggests the particular combinations of herb and flower extracts or extracts any other naturally occurring sources as in the present application. In particular, none of these publications describes or suggests the particular combinations of such extracts and natural sources as in the present application, especially their particular relative and absolute amounts in any pharmaceutically acceptable composition. Most particularly, none of these publications describes or suggests the effective amounts of the naturally occurring active compounds in such extracts and their effective pharmaceutical activity as in the present invention. Further, these publications do not describe or suggest synergistic, amplifying and/or complementing effects in particular combinations of the extracts and their corresponding effective compounds as in the present invention.

It is, therefore, an object of the present invention to provide pharmaceutically acceptable compositions that comprise effective amounts of combinations of extracts of herbs, flowers and any other naturally occurring sources for treating, preventing, inhibiting and ameliorating conditions and disorders in a person. Particularly, such conditions are related to inflammation, in particular chronic inflammation.

It is still another object of the present invention to provide a method of producing such pharmaceutically acceptable compositions.

It is still another object of the present invention to provide a method of treating disorders, conditions and diseases and regimens of pharmaceutical treatment thereof. More particularly, the present invention provides pharmaceutical compositions and corresponding treatment regimens for treating inflammation related to or initiated by diabetes, high levels of blood glucose and viral infections with such pharmaceutically acceptable compositions.

It is still another object of the invention to provide a method of treating disorders, diseases and conditions, having a correlation with chronic inflammation, including, but not limited to, cardiovascular diseases, cancers, allergies, obesity, diabetes, hypoglycemia, digestive system diseases, liver disease, including e.g. fatty liver disease and cirrhosis of the liver chronic liver disease, jaundice, ascites, varices, alcoholic liver disease, non-alcoholic liver disease etc., gall stones, degenerative diseases, auto-immune disorders, anemia, sickle cell anemia, fibromyalgia, and Alzheimer's disease or other related dementia e.g., vascular dementia, mixed dementia, fronto-temporal dementia, Lewy-body dementia, memory loss, cholesterol disorders, hair loss, depression, hormonal disorders, PMS (premenstrual Syndrome), Menopause Symptoms, PCOS (Polycystic ovary syndrome), sleep disruptions/disorders, headaches, loss of weight, overweight, loss of appetite, vertigo, dizziness, lack of focus, erectile disfunction, fatigue and weakness, muscle pain, diarrhea, loss of smell, loss of taste, skin rash, back pain, chest pain, coughing, runny nose, sore throat, nausea, vomiting, fever, eye infection, ear infection, Schizophrenia, delusions, hallucinations, paranoia, disorganized speech, Autism, PTSD, Aspergers, ADD, ADHD, depression, anxiety, delliurm, glaucoma, fibrotic formation/function in the cornea, reactive attachment disorder, intellectual disability, Neurodegenerative diseases, including Amyotrophic lateral sclerosis, (ALS, Lou Gehrig's disease), Charco-Marie-Tooth disease, Chronic traumatic encephalopathy, cystic fibrosis, cytochrome c oexidase, Ehlers-Danlos syndrome, Fibrodysplais ossificans progressive, Friedreich's ataxis, Frontotemporal deminatia, Huntington disease, infantile neuroaxonal dystrophy, keratoconus, Leukodystrophies, maculardegeneration, marfans syndrome, mitochondrial myppathies and mitochondrial DNA depletion syndrome, multiple sclerosis, multiple system atrophy, muscular dystrophies, neuronal ceroid lipfuscinosis, Niemann-Pick diseases, osteoarthritis, osteoporosis, pulmonary arterial hypertension, all prion diseases, progressive supranuclear palsy, retinitis pigmentosa, rheumatoid arthritis, Sandoff diseases, spinal muscular atrophy, substance use disorder, subacute sclerosing, Tay-Sachs disease, Parkinson disease, essential tremor, Parkinsonism, NPH (Normal pressure hydrocelphalus), aging disorders, aging and skin damage including skin wounds, skin scarring, loosening of the skin and darker pigmentation spots forming on the skin.

This and other objects, embodiments, compositions, diseases and disorders that are treatable with such compositions and methods of producing them will become apparent as the descriptions proceeds.

### SUMMARY OF THE INVENTION

According to an aspect of the invention there is provided a method of preventing or treating an inflammatory disease in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating inflammation and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an aspect of the invention there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of ameliorating inflammation and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating an inflammatory disease.

According to an aspect of the invention there is provided a composition of matter comprising at least 2 of a plant species or genus thereof-derived components selected from the group consisting of a plant part, extract thereof, fraction thereof, active ingredient thereof, synthetic analog thereof, mimetic thereof or combination thereof, wherein the component is capable of ameliorating inflammation and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an aspect of the invention there is provided a food supplement comprising a combination of at least 2 of a plant species or genus thereof-derived component selected from the group consisting of a plant part, extract thereof, fraction thereof, active ingredient thereof, synthetic analog thereof, mimetic thereof or combination thereof, wherein the component is capable of ameliorating inflammation and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an aspect of the invention there is provided a food supplement, composition or extracts further including “Beduin Tea” comprising Rose Leaves Micromeria fruticose, Salvia, cymbopgon (Citral,) Aloysia, verbena officinalis, origanum majorana, and menthe.

According to an aspect of the invention there is provided a food supplement, composition or extracts further including “Beduin Tea” comprising Thyme, sage, cardamom, cinnamon, black tea, habuk, Marmaya.

According to some embodiments, the component comprises at least 2 components from the above-listed plant species.

According to some embodiments, the component comprises at least 3 components from the above-listed plant species.

According to some embodiments, the component comprises at least 4 components from the above-listed plant species.

According to some embodiments, the component comprises at least 5 components from the above-listed plant species.

According to some embodiments, the component comprises 5-10 components from the above-listed plant species.

According to some embodiments, the component comprises thymoquinone or an analog thereof.

According to some embodiments, the component comprises thymol or an analog thereof.

According to some embodiments, the component comprises carvacrol or an analog thereof.

In some embodiments of the present invention the component comprises tryptophan, analogs of tryptophan or extract of plants containing tryptophan such as sesame or oregano.

According to some embodiments, the inflammatory disease comprises an autoimmune disease.

According to some embodiments, the inflammatory disease comprises an acute inflammatory disease.

According to some embodiments, the inflammatory disease comprises an autoimmune disease.

According to some embodiments, the inflammatory disease comprises diabetes.

According to some embodiments, the diabetes comprises type I diabetes.

According to some embodiments, the diabetes comprises type II diabetes.

According to some embodiments, the diabetes comprises gestational diabetes.

According to some embodiments, the invention causes a reduction in C-Reactive protein.

According to some embodiments, the invention inhibits ACE2 binding by coronaviruses.

According to some embodiments, the invention reduces and/or eliminates allergic responses, including, but not limited to, allergens from animals (including dogs, cats, horses, poultry etc.), dust, tree nuts, peanuts, eggs, milk, trees, grasses, flowers, etc.)

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C shows embodiments in plant extraction methods as taken from berkem (dot) com. FIG. 1A—scheme describing the general principle of plant extraction; FIG. 1B—scheme describing the main separation process according to some embodiments; FIG. 1C—scheme describing parameters that may influence the process.

FIG. 2—depicting SDS-page of the SARS-COV-2 S1 subunit protein digestion assay with the tested extracts following an incubation time of 6 h at 37° C.

FIG. 3—depicting SDS-page of the SARS-COV-2 S2 subunit protein digestion assay with the tested extracts following an incubation time of 6 h at 37° C.

FIG. 4—depicting SDS-page of the SARS-COV-2 Nucleocapsid digestion assay with the tested extracts following an incubation time of 6 h at 37° C.

FIG. 5—depicting a graphic representation of the densitometry test of the SARS-CoV-2 S1 subunit protein digestion assay with the tested extracts following an incubation time of 6 h at 37° C.

FIG. 6—depicting a graphic representation of the densitometry test of the SARS-CoV-2 S2 subunit protein digestion assay with the tested extracts following an incubation time of 6 h at 37° C.

FIG. 7—depicting a graphic representation of the densitometry test of the SARS-CoV-2 Nucleocapsid digestion assay with the tested extracts following an incubation time of 6 h at 37° C.

### DESCRIPTION OF SPECIFIC EMBODIMENTS OF THE INVENTION

The present invention, in some embodiments thereof, relates to compositions and methods for treating or preventing inflammatory diseases, in particular chronic inflammatory diseases, as well as disease and disorders associated with inflammation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Inflammatory diseases and autoimmune diseases exert a devastating personal and economic burden. Inflammatory diseases occur when an inflammatory response is initiated that is inappropriate and/or does not resolve in the normal manner but rather persists and results in a chronic inflammatory state, which is cureless.

Thus, according to an aspect of the invention, there is provided a method of preventing or treating an inflammatory disease in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein said component is capable of ameliorating inflammation and wherein said plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an additional or an alternative aspect there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein said component is capable of ameliorating inflammation and wherein said plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating an inflammatory disease.

According to an additional or an alternative aspect there is provided a composition of matter comprising at least 2 of a plant species or genus thereof-derived components selected from the group consisting of a plant part, extract thereof, fraction thereof, active ingredient thereof, synthetic analog thereof, mimetic thereof or combination thereof, wherein said component is capable of ameliorating inflammation and wherein said plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an additional or an alternative aspect there is provided a food supplement comprising a combination of at least 2 of a plant species or genus thereof-derived component selected from the group consisting of a plant part, extract thereof, fraction thereof, active ingredient thereof, synthetic analog thereof, mimetic thereof or combination thereof, wherein said component is capable of ameliorating inflammation and wherein said plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an aspect of the present invention there is provided compositions or food supplements comprising Bromelain or pineapple extracts comprising Bromelain.

As early as 1899 H. F. Harris have reported “A case of diabetes mellitus quickly following Mumps”. A significant number of viruses have been associated with type 1 diabetes, including enteroviruses such as Coxsackievirus B (CVB), but also rotavirus, mumps virus, and cytomegalovirus. Rubella virus has been suggested to cause type 1 diabetes (see Filippi and. Von Herrath, 2008). The combined incidence of diabetes and latent diabetes in this group of patients was nine out of forty-four (20%). These findings suggest a causative relationship between congenital rubella infection and diabetes mellitus. Further more, epidemiological studies have suggested a linkage between type 2 diabetes and chronic hepatitis C virus (HCV) infection (Yoshizumi Shinatai et. Al, 2005).

It is acknowledged herein that the glycoproteins that are on the surfaces of many viruses, including coronavirus, help them to bind to host cells. There are sugars/glycans on the surface of the Coronavirus Spike Protein (“Sugary Camouflage on Coronavirus offers vaccine clues” Sugars on Coronavirus Spike Protein Offer Vaccine Clues | Quanta Magazine).

Tryptophan is important in glycan-protein interaction (“The Sugar Code”). The study of glycan-protein interactions provides insight into the mechanism of cell signaling and allows to create better-diagnosing tools for many diseases including cancer. “Indeed there are no known types of cancer that do no not involve erratic patterns of protein glycosylation. The sweet spot: defining virus-sialic acid interactions” | Nature Reviews Microbiology.

It is further herein acknowledged that SGLT2 inhibitor drugs are diabetes medications. In order for glucose to reach cells in the human body, there are proteins on the cell membranes that are carriers of glucose and sodium. These carrier proteins are called Sodium-Glucose Transport proteins. There are about five types of SGLT, with the kidneys having a protein from this family called SGLT2. This protein is responsible for nearly 90% reuptake of kidney glucose [2]. Knowing this mechanism was the groundwork for the formation of the drugs that inhibit SGLT2. SGLT2 inhibitors inhibit the protein responsible for absorbing glucose from urine into the blood and cause:
Secretion of glucose in the urine
Lowering blood glucose levels

Tryptophan is important in glycan-protein interaction, as explained above. Thus, according to an aspect of the invention, there is provided a method of reducing the infectivity of a diabetes mediated virus by modifying the viral entry mechanism proteins in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein said component is capable of attenuating viral entry and wherein said plant species is selected from the group consisting of Nigella sativa, Thymus capitatus, Thymus vulgaris, Origanum syriacum, Thymbra spicata, Satujera thymbra,

Sesamum indicum, Rhus coriaria, Gynostemma pentaphyllum, Boswellia sacra and Panax ginseng.

It is further herein acknowledged that, in some embodiments of the present invention, Tryptophan, or tryptophan analogues or extracts of plants containing tryptophan are used in the method of treatment or composiitons of the present invention.

According to an aspect of the invention there is provided a food supplement, composition or extracts further including “Beduin Tea” comprising Rose Leaves Micromeria fruticose, Salvia, cymbopgon (Citral), Aloysia, verbena officinalis, origanum majorana, and/or menthe.

According to an aspect of the invention there is provided a food supplement, composition or extracts further including “Beduin Tea” comprising Thyme, sage, cardamom, cinnamon, black tea, habuk, Marmaya.

The term “plant” as used herein encompasses whole plants, a grafted plant, ancestors and progeny of the plants and plant parts, including seeds, flowers, bark, shoots, stems, roots (including tubers), fruit, rootstock, scion, and plant cells, tissues and organs.

According to a specific embodiment, the plant part is a seed.

According to a specific embodiment, the plant part is a fruit.

According to a specific embodiment, the plant part is a leaf.

According to a specific embodiment, the plant part is a stem.

According to a specific embodiment, the plant part is a flower.

The plant part can be a solid part or a non-solid part such as oil or aqueous portions of the plant.

The plant may be in any form including suspension cultures, embryos, meristematic regions, callus tissue, leaves, gametophytes, sporophytes, pollen, and microspores.

The term plant refers to a wild plant or a cultivated variety thereof.

As used herein the term “plant species” refers to a sub-group of one or more plants within the genus. These plants will share similar characteristics with each other. There may be a single plant within a species, or there may be many hundreds of plants. The term intends to include subspecies, such as grown or can be found in different geographical location, e.g., Lebanese Sumac and Syrian Sumac or Korean Ginseng and American Ginseng.

As used herein “plant genus” refers to a taxonomic rank below family and above species.

It will be appreciated that the relevant species and genera and listed below and each option or combination thereof represents a different embodiment of the invention.

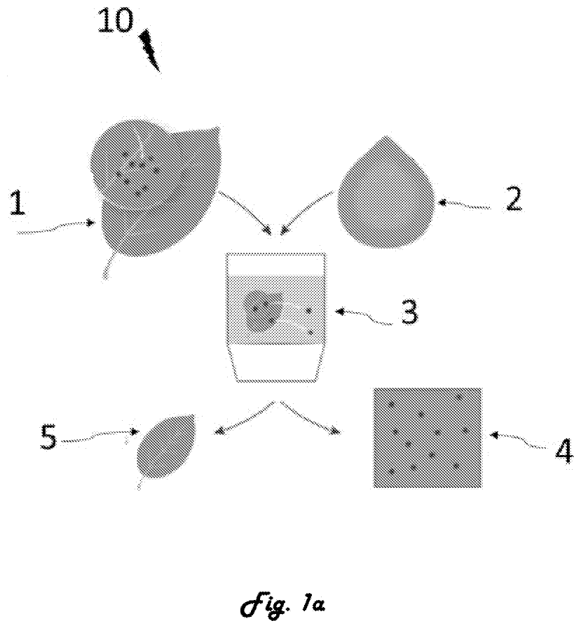

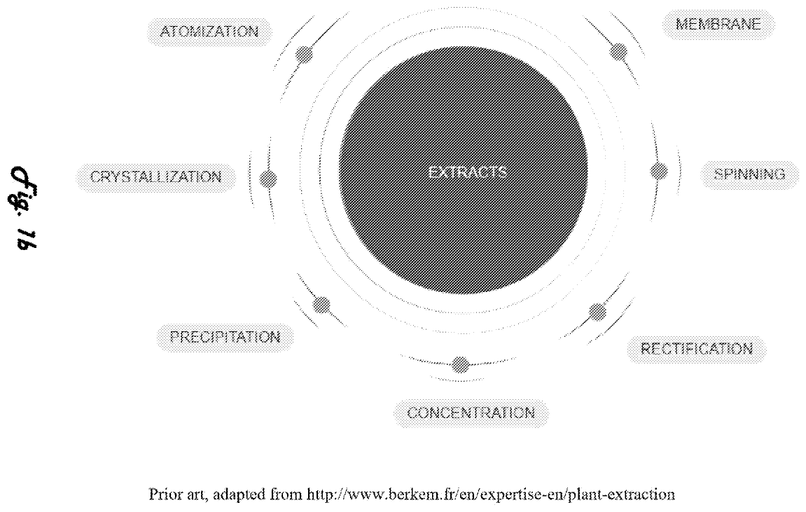

The term ‘extraction” refers to a separation process which relies on the separation of one or more analytes from the components of a sample other than the one or more analytes. Extractions are processes that typically use two immiscible phases to separate one or more solutes from one phase into the other. The distribution of a solute between two phases is an equilibrium condition described by partition theory. For example, boiling tea leaves in water extracts the tannins, theobromine, and caffeine out of the leaves and into the water. More typical extractions preformed typically but not only in a laboratory are settings of organic compounds out of an aqueous phase and into an organic phase. Common extractants are arranged from ethyl acetate to water (ethyl acetate<acetone<ethanol<methanol<acetone: water (7:3)<ethanol: water (8:2)<methanol: water (8:2)<water) in increasing order of polarity according to the Hildebrand solubility parameter. Procedures for plant extraction are provided in FIGS. 1A-C.

The term “extract” as used herein refers to the result of such process of separation that can take the form of a solution formulation or other chemical form depending on the extraction process. In particular, the term extract can relate to a substance made by extracting a part of a sample (e.g. a raw material), such as by using a solvent such as ethanol or water. In various instances an extract relates to a solvent that is enriched in one or more solute. In particular, a “plant extract” in the sense of the present disclosure typically comprises a concentrated preparation of a plant material obtained by isolating or purifying desired active constituents with one or more extraction processes.

The choice of the solvent depends on the desired component to be obtained. For example, to extract polar components in an extraction process suggested solvents include, but are not limited to, water, ethanol methanol or butanol while for non polar compounds diethyl ether, hexane or chloroform depending on the use of the extract. For midpolar one may choose Ethyl acetate but other solvants can be used as well.

The general procedure of solid/liquid extraction can be scaled in five different ways:

Maceration: the contact stage is maintained at room temperature.

Decoction or reflux: the contact stage is maintained at the boiling point of the solvent.

Digestion: the contact stage is maintained at a temperature in between those of the previous two cases.

Infusion: the boiling solvent is poured over the solid, then left to cool for a set time.

Leaching or percolation: the solvent passes through the biomass.

It is also possible to combine these methods with each other or with other processes such as distillation, steam distillation, rectification, etc.

According to another embodiment, the use of various solvents, either successively or in combination is contemplated and the ordinary skilled of organic chemistry will know which to choose according to the active ingredient as described below.

Extraction may be further assisted by other means such as ultrafiltration, reverse osmosis, high pressure (supercritical CO₂), microwaves, ultrasound, etc.

In some embodiments, the plant part is contacted with a polar solvent (e.g. ethanol) or nonpolar solvent (e.g., hexane or pentane) for several minutes, e.g., 15 minutes or more, about 30 minutes or more, about 1 hour or more, about 2 hours or more, or about 5 hours or more.

Temperature can also be controlled during the contacting.

According to specific embodiments, the plant part is contacted with the solvent (e.g. ethanol) while being constantly mixed e.g. on a shaker.

It will be appreciated that the extraction process can also be solvent-free.

For example, solvent-free microwave extraction (SFME) has been proposed as a green method for the extraction of essential oil from aromatic herbs that are extensively used in the food industry. This technique is a combination of microwave heating and dry distillation performed at atmospheric pressure without any added solvent or water. The isolation and concentration of volatile compounds is performed in a single stage. In some embodiments, SFME and/or hydro-distillation (HD)), are used for the extraction of essential oil from the plants of the invention.

In some embodiments, the process of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According specific embodiments, the present invention contemplates drying (i.e. removal of the polar/non-polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment the polar solvent is evaporated with a vacuum evaporator.

The selection of the extraction process much depends on the component to be isolated.

It will be appreciated that following generation of the extract, specific embodiments of the present invention further contemplate additional purification steps so as to further isolate/purify active agents from the extract, for example, by fractionating the filtered extract.

in As used herein “a fraction” refers to a portion of the extract that contains only certain chemical ingredients of the extract but not all.

Fractionating can be performed by processes such as, but not limited to: column chromatography, preparative high performance liquid chromatography (“HPLC”), reduced pressure distillation, and combinations thereof.

According to a specific embodiment, fractionating is performed by HPLC.

In some embodiments, fractionating comprises re-suspending the filtered extract in a polar solvent (such as methanol, as discussed above), applying the polar extract to a separation column, and isolating the extract having the anti-respiratory disease (e.g. anti-fibrotic, anti-inflammatory) activity by column chromatography (preparative HPLC).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N, N-dimethylacetamide, and the like, and combinations thereof.

According to an alternative or an additional embodiment, liquid chromatography comprises high performance liquid chromatography (HPLC).

According to an alternative or an additional embodiment, liquid chromatography is performed on a reverse stationary phase.

The fractions may be characterized by analytical methods such as, but not limited to, spectroscopic methods such as, but not limited to, ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

The component (active ingredients, extract and/or fractions) obtained may be tested for ameliorating inflammation. Exemplary methods for testing the effect are further described herein below.

For example, there are a few so-called markers that help diagnose inflammation in the body. These include, but are not limited to: Serum protein electrophoresis (SPE), C-reactive protein (CRP), Erythrocyte sedimentation rate (ESR), plasma viscosity and more.

Alternatively or additionally, inflammation can be determined at the whole body level (e.g., by the determination of fecer, sweating, swelling, fever, redness).

However, to be mre accurate, the inflammation should be determined using a test specific for the disease. For instance, MRI in multiple sclerosis (MS), or molecular markers which are specific for MS, or in rheumatoid arthritis joint pain, swelling, stiffness, or loss of joint function.

The active ingredients, extract and/or fraction described herein may be immediately used or stored until further used.

According to specific embodiments, the active ingredients, extract and/or fractions is kept frozen, e.g. in a freezer, until further use (e.g. at about −20° C. to-90° C., at about −70° C. to −90° C., e.g. at −80° C.), for any required length of time.

According to other specific embodiments, the active ingredients, extract and/or fractions is immediately used (e.g. within a few minutes e.g., up to 30 minutes).

The active ingredients, extract and/or fractions may be used separately. Alternatively, different active ingredients, extract and/or fractions (e.g. from different plants or from separate extraction procedures) may be pooled together. Likewise, different active ingredients, extract and/or fractions (from the same extract, from different extracts, from different plants and/or from separate extraction procedures) may be pooled together.

Using the present teachings, the present inventor was able to identify not only plants and extracts that can be used to effectively treat or prevent inflammation, but also active ingredients thereof.

“Active ingredient” refers to a defined chemical composition which is responsible for the anti (preventive or therapeutic) effect against inflammation.

The active ingredient can be purified from a plant or chemically synthesized (artificial, man-made).

Also contemplated herein are analogs and derivatives of the active ingredients as long as the anti-inflammation effect is maintained, which are also referred to as mimetics.

Following are some non-limiting examples for extraction of active ingredients from selected plants of the present invention.

Extraction from leaves of T. capitatus—The Aerial parts of T. capitatus (leaves) samples are collected. Leaves separated from branches are dehydrated at room temperature for 7 days and slightly blended into fine powders for extractions.

Essential oil (EO) extraction-hydro-distillation is used to extract EO from the plant, e.g., dried aerial parts of T. capitatus. In brief, the extraction is conducted for several hours for example, 3 h, by mixing 100 g of plants in 500 mL of distilled water. The extract is dried and concentrated using sodium sulphate and rotatory evaporator under reduced pressure. The EO yield is established by quantity of the obtained oil in mL for 100 g of dried plant. Finally, the pure EO os stored at −4° C. until further analyzed.

Essential oil analysis—The chemical composition of EO is examined by GC and GC-MS. GC analysis is conducted using gas chromatograph. The proportion of the constituents is determined by the integration of peak areas. In addition, mass spectrometry (MS) can be used to analyze the EO typically under the same conditions as described above for gas chromatography. The identification of the different compounds is defined by comparison of their retention indexes (determined relatively to the retention times of a series of n-alkanes) with those of standards of the Wiley library search routines12, based on fit and purity of mass spectra. Such conditions are used for determining the active ingredients as described below.

Extraction from Satujera Thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil is dried using anhydrous magnesium sulfate and stored at 4° C. S. thymbra oil was analyzed by GC/MS.

Extraction from Rhus coriaria (Sumac)

In order to isolate, determine and identify the compounds from the Rhus coriaria fruits, different extracts are taken from the fruit or leaves of the Sumac plant. Some are isolated from aqueous extracts, others from alcoholic extracts and some from lipid extracts. Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. Analysis of the chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Extraction of thymoquinone from Nigella sativa

Various methods can be used including microwave-assisted extraction system having temperature controlling function as well as other extraction methods, Soxhlet and conventional solid/liquid extraction.

### Nigella Sativa

| CHEMICAL COMPOUND | Plant Part | Low ppm | High ppm |
|---|---|---|---|
| 24-ETHYL-LOPHENOL | Seed Oil | | |
| 24-METHYL-LOPHENOL | Seed Oil | | |
| 24-METHYLENE-CYCLOARTANOL | Seed Oil | | |
| 5-DEHYDRO-AVENASTEROL | Seed Oil | | |
| 7-DEHYDRO-AVENASTEROL | Seed Oil | | |
| ALANINE | Seed | 8,000 | 10,255 |
| ALPHA-SPINASTEROL | Seed Oil | | |
| | Seed | | |
| ARABIC-ACID | Seed | | |
| ARACHIDIC-ACID | Seed Oil | | 1,900 |
| ARACHIDONIC-ACID | Seed Oil | | 24,900 |
| ARGININE | Seed | 41,500 | 53,050 |
| ASCORBIC-ACID | Leaf | | 2,577 |
| ASH | Seed | 38,000 | 53,000 |
| ASPARAGINE | Seed | | 0 |
| ASPARTIC-ACID | Seed | 10,670 | 13,650 |
| ASTRAGALIN | Seed | | 200 |
| BETA-AMYRIN | Seed Oil | | |
| BETA-SITOSTEROL | Seed Oil | | |
| | Seed | | 3,218 |
| BUTYROSPERMOL | Seed Oil | | |
| CALCIUM | Seed | | 10,600 |
| CAMPESTANOL | Seed Oil | | |
| CAMPESTEROL | Seed Oil | | |
| | Seed | | |
| CARBOHYDRATES | Seed | | 339,600 |
| CARVONE | Seed Essent. | | |
| | Oil | | |
| | Seed | 2,250 | 9,600 |
| CHOLESTEROL | Seed Oil | | |
| | Seed | | |
| CITROSTADIENOL | Seed Oil | | |
| CYCLOARTENOL | Seed Oil | | |
| CYCLOEUCALENOL | Seed Oil | | |
| CYMENE | Seed | | |
| CYSTINE | Seed | | |
| D-LIMONENE | Seed | | |
| DAMASCENINE | Plant | | |
| DEHYDROASCORBIC-ACID | Leaf | | 295 |
| DITHYMOQUINONE | Seed Oil | | |
| EICOSADIENOIC-ACID | Seed Oil | | 25,000 |
| | Seed | 8,979 | 10,525 |
| EO | Seed Oil | | 4,500 |
| | Seed | 4,000 | 16,000 |
| FAT | Seed | 354,900 | 416,000 |
| FIBER | Seed | | 55,000 |
| FIXED-OIL | Seed | | 380,000 |
| GLUCOSE | Seed | | |
| GLUTAMIC-ACID | Seed | 28,085 | 35,900 |
| GLYCINE | Seed | 8,840 | 20,700 |
| GRAMISTEROL | Seed Oil | | |
| HEDERAGENIN | Seed | | 10,000 |
| INDOLE-3-ACETIC-ACID | Tissue Culture | | 14 |
| IRON | Seed | | 140 |
| ISOLEUCINE | Seed | 8,570 | 10,960 |
| KAEMPFEROL-3-O-BETA- | Seed | | 100 |
| GLUCOPYRANOSYL-(1,2)-O-BETA- | | | |
| GALACTOPYRANOSYL-(1,2)-BETA- | | | |
| GLUCOPYRANOSIDE | | | |
| LEUCINE | Seed | 23,130 | 29,595 |
| LINOLEIC-ACID | Seed Oil | 487,600 | 576,300 |
| | Seed | 128,124 | 233,459 |
| LINOLENIC-ACID | Seed Oil | 5,700 | 7,000 |
| | Seed | 2,484 | 2,912 |
| LIPASE | Seed | | |
| LOPHENOL | Seed Oil | | |
| LYSINE | Seed | 16,200 | 20,700 |
| MELANTHIGENIN | Seed | | |
| MELANTHIN | Plant | | 15,000 |
| | Seed | | 15,000 |
| METHIONINE | Seed | 13,100 | 16,750 |
| MYRISTIC-ACID | Seed Oil | 1,900 | 18,000 |
| | Seed | 567 | 1,082 |
| NIGELLICINE | Seed | | |
| NIGELLIDINE | Seed | | |
| NIGELLIMINE | Seed | | 0.13 |
| NIGELLIMINE-N-OXIDE | Seed | | 0.2 |
| NIGELLIN | Seed | | |
| NIGELLINE | Seed Essent. | | |
| | Oil | | |
| NIGELLONE | Seed Essent. | | |
| | Oil | | |
| | Plant | | |
| | Seed | | |
| OBTUSIFOLIOL | Seed Oil | | |
| OLEIC-ACID | Seed Oil | 244,600 | 262,400 |
| | Seed | 89,911 | 184,912 |
| PALMITIC-ACID | Seed Oil | 120,000 | 171,200 |
| | Seed | 22,464 | 50,523 |
| PALMITOLEIC-ACID | Seed Oil | | 2,000 |
| PHENYLALANINE | Seed | 16,850 | 21,560 |
| PHYTOSTEROLS | Seed | | 5,100 |
| POTASSIUM | Seed | | 5,820 |
| PROLINE | Seed | 11,350 | 14,520 |
| PROTEIN | Seed | 210,000 | 271,900 |
| QUERCETIN-3′-GLUCOSIDE | Seed | | 440 |
| QUERCETIN-3-O-(6-FERULOYL-BETA- | Seed | | 240 |
| GLUCOPYRANOSYL)-(1,2)-BETA- | | | |
| GALACTOPYRANOSYL-(1,2)-BETA- | | | |
| GLUCOPYRANOSIDE | | | |
| QUERCETIN-3-O-BETA- | Seed | | 1,380 |
| GLUCOPYRANOSYL-(1,2)-O-BETA- | | | |
| GALACTOPYRANOSYL-(1,2)-BETA- | | | |
| GLUCOPYRANOSIDE | | | |
| RESIN | Seed | | |
| RUTIN | Seed | | 200 |
| SERINE | Seed | 4,210 | 5,385 |
| SODIUM | Seed | | 980 |
| STEARIC-ACID | Seed Oil | 18,100 | 60,400 |
| | Seed | 8,722 | 10,192 |
| STIGMAST-7-EN-3-BETA-OL | Seed Oil | | |
| STIGMASTANOL | Seed Oil | | |
| STIGMASTEROL | Seed Oil | | |
| | Seed | | |
| TANNIN | Seed | | |
| TARAXEROL | Seed Oil | | |
| TELFAIRIC-ACID | Seed Oil | | |
| THREONINE | Seed | 2,615 | 3,345 |
| THYMOHYDROQUINONE | Seed | | |
| THYMOL | Seed Oil | | |
| THYMOQUINONE | Seed Essent. | | |
| | Oil | | |
| | Seed Oil | | |
| | Seed | | |
| TIRUCALLOL | Seed Oil | | |
| TRYPTOPHAN | Seed | | |
| TYROSINE | Seed | 12,925 | 16,530 |
| VALINE | Seed | 6,500 | 8,325 |

According to a specific embodiment, active ingredients (e.g., which can be obtained by supercritical carbon dioxide extraction method) include but are not limited to:

| Compound | RIₑₓₚ | RIₗᵢₜ | SFE 1 | SFE 2 | HD SF [symbol] | Identification |
|---|---|---|---|---|---|---|
| n-Nonane^{a} | 905 | 900 | 0.12 | — | — | RI, MS |
| Tricyclene | 926 | 926 | tr | — | — | RI, MS |
| Camphene | 953 | 953 | — | — | 1.64 | RI, MS |
| β-Pinene | 958 | 959 | — | — | 0.40 | RI, MS |
| 2,4,(10)-Thujadiene | 967 | 960 | 4.74 | 0.19 | — | RI, MS |
| Sabinene | 978 | 977 | 1.05 | — | — | RI, MS |
| β-Myrcene | 990 | 991 | 0.31 | — | — | RI, MS |
| 1,8-Cineole | 1013 | 1010 | — | — | 0.98 | RI, MS |
| α-Terpinene | 1025 | 1026 | 2.34 | — | — | RI, MS |
| Limonene | 1034 | 1034 | 0.18 | 0.38 | 1.03 | RI, MS |
| γ-Terpinene | 1054 | 1056 | 27.46 | 13.20 | 12.87 | RI, MS |
| cis-Sabinene hydrate | 1063 | 1068 | — | 0.38 | Tr | RI, MS |
| allo-Ocimenol^{a} | 1079 | 1071 | — | 0.11 | — | RI, MS |
| Linalool | 1087 | 1080 | 0.25 | 0.19 | — | RI, MS |
| Terpinolene | 1091 | 1088 | — | — | Tr | RI, MS |
| trans-Sabinene hydra [symbol] | 1099 | 1097 | 0.37 | — | — | RI, MS |
| Terpinen-1-ol^{a} | 1124 | 1120 | — | — | 0.11 | RI, MS |
| 1,5,8-p-Menthatriene^{a} | 1130 | 1135 | 0.43 | 0.38 | — | RI, MS |
| Borneol | 1152 | 1152 | — | — | 1.02 | RI, MS |
| Pinocarvone | 1167 | 1165 | 2.96 | 3.00 | — | RI, MS |
| trans-Dihydrocarvone [symbol] | 1208 | 1202 | — | 0.19 | — | RI, MS |
| Dihydrocarvone^{a} | 1215 | 1214 | 0.37 | 2.06 | — | RI, MS |
| Ocimenone (E)^{a} | 1249 | 1239 | 1.54 | 1.50 | — | RI, MS |
| Thymoquinone | 1250 | 1250 | 35.05 | 33.12 | 38.41 | RI, MS, NMR |
| Thymol | 1283 | 1288 | 7.43 | 5.30 | 16.95 | RI, MS, NMR |
| Carvacrol | 1299 | 1299 | 1.98 | 1.73 | 0.81 | RI, MS |
| 2-Undecanone | 1312 | 1315 | — | — | 13.72 | RI, MS |
| n-Octyl isobutyrate^{a} | 1323 | 1326 | — | — | 0.12 | RI, MS |
| α-Longipinene | 1330 | 1334 | 0.26 | — | — | RI, MS |
| Citronellyl acetate^{a} | 1339 | 1336 | — | — | 0.50 | RI, MS |
| Thymohydroquinone | 1353 | 1351 | — | — | Tr | RI, MS |
| methyl ether^{a} | | | | | | |
| Cyclosativene | 1367 | 1366 | — | — | 1.43 | RI, MS |
| α-Longicyclene | 1381 | 1380 | 0.43 | 5.25 | — | RI, MS |
| α-Copaene | 1385 | 1383 | 1.54 | 2.00 | 0.41 | RI, MS |
| α-Longifolene | 1391 | 1387 | — | — | 0.51 | RI, MS |
| (Z)-Caryophyllene^{a} | 1395 | 1395 | 0.23 | — | — | RI, MS |
| β-Caryophyllene | 1420 | 1417 | 2.89 | 5.07 | 4.80 | RI, MS |
| Thymohydroquinone | 1429 | 1425 | 0.43 | — | — | RI, MS |
| dimethylether^{a} | | | | | | |
| Aromadendrene^{a} | 1437 | 1438 | — | — | 1.04 | RI, MS |
| Thymohydroquinone | 1515 | 1509 | 1.17 | 1.12 | 2.31 | RI, MS, NMR |
| Davanone^{a} | 1587 | 1586 | 0.31 | — | — | RI, MS |
| 8-Heptadecene^{a} | 1683 | 1680 | 1.23 | 1.13 | 0.86 | RI, MS |
| Dihydrofarnesyl aceta [symbol] | 1841 | 1840 | 2.28 | 4.69 | — | RI, MS |
| Pimaradiene^{a} | 1934 | 1935 | 1.23 | 2.25 | — | RI, MS |
| Palmitic acid | 1947 | 1946 | 0.18 | — | — | RI, MS |
| Pimara-8(14),15-diene | 1968 | 1966 | 0.92 | — | — | RI, MS |
| Octadecanoic acid | 2145 | 2157 | 0.26 | 12.31 | — | RI, MS |
| Total identified | | | 99.94 | 95.55 | 99.92 | |

Grouped compounds:

| | | | |
|---|---|---|---|
| Quinones | 44.08 | 39.54 | 57.67 |
| Monoterpene hydrocarbons | 36.51 | 14.15 | 15.94 |
| Oxygenated monoterpenes | 7.47 | 9.16 | 17.14 |
| Sesquiterpene hydrocarbons | 5.35 | 12.32 | 8.19 |
| Oxygenated sesquiterpenes | 2.59 | 4.69 | — |
| Diterpenes | 2.15 | 2.25 | — |
| Alkane | 0.12 | — | — |
| Alkenes | 1.23 | 1.13 | 0.86 |
| Fatty acids | 0.44 | 12.31 | — |
| Fatty acid esters | — | — | 0.12 |

[symbol]

indicates data missing or illegible when filed

Additional plants that are contemplated herein are of the genus Nigella.

Nigella is a genus of 18 species of annual plants in the family Ranunculaceae, native to Southern Europe, North Africa, South Asia, Southwest Asia and Middle East. Common names applied to members of this genus are nigella, devil-in-a-bush or love-in-a-mist.
Nigella arvensis
Nigella carpatha
Nigella damascena
Nigella degenii
Nigella deserti
Nigella doerfleri
Nigella elata
Nigella fumariifola
Nigella hispanica
Nigella latisecta
Nigella nigellastrum
Nigella orientalis
Nigella oxypetala
Nigella papillosa
Nigella sativa
Nigella segetalis
Nigella stricta
Nigella unguicularis

According to a specific embodiment the active ingredient is thymoquinone.

Additional plants containing thymoquinone include, but are not limited to:
Monarda fistulos (of the genus Monarda);
Satureja montana (of the genus Satujera);

Additional families containing thymoquinone include, but are not limited to: Asteraceae—examples include, but are not limited to the subfamilies:
Barnadesioideae Bremer & Jansen
Carduoideae Sweet
Cichorioideae Chevallier
Corymbioideae Panero & Funk
Famatinanthoideae S. E. Freire, Ariza & Panero
Gochnatioideae Panero & Funk
Gymnarrhenoideae Panero & Funk
Hecastocleidoideae Panero & Funk
Mutisioideae Lindley
Pertyoideae Panero & Funk
Stifftioideae Panero
Wunderlichioideae Panero & Funk

Cupressaceae.
Cunninghamioideae
Taiwanioideae
Athrotaxidoideae
Sequoioideae
Taxodioideae
Callitroideae
Cupressoideae
Incertae sedis

Lamiacea

Ranunculacea
Hydrastidoideae
Glaucidioideae
Coptoideae
Thalictroideae
Ranunculoideae

List of plants that contain Carvacrol include, but are not limited to:
Monarda didyma
Nigella sativa
Origanum compactum
Origanum dictamnus
Origanum microphyllum
Origanum onites
Origanum scabrum
Origanum syriacum
Origanum vulgare
Plectranthus amboinicus
Thymus glandulosus
Lavandula multifida
Origanum minutiflorum
Satureja thymbra

Active ingredients found in Thymus Capitatus

| No | RI | Compound | % |
|---|---|---|---|
| 1 | 935 | α-Thujene | 0.54 |
| 2 | 940 | α-Pinene | 0.38 |
| 3 | 991 | Myrcene | 0.87 |
| 4 | 1019 | α.-terpinene | 1.11 |
| 5 | 1025 | p-Cymene | 6.25 |
| 6 | 1063 | γ-Terpinene | 6.75 |
| 7 | 1089 | α-terpinolene | 0.26 |
| 8 | 1101 | Linalool | 1.51 |
| 9 | 1179 | Terpinen-4-ol | 1.40 |
| 10 | 1185 | 4-Carvomenthenol | 0.94 |
| 11 | 1260 | Geraniol | 0.25 |
| 12 | 1309 | Carvacrol | 65.38 |
| 13 | 1310 | Thymol | 1.35 |
| 14 | 1358 | Eugenol | 0.21 |
| 15 | 1408 | Carvacryl Acetate | 0.45 |
| 16 | 1427 | β-Caryophyllene | 4.94 |
| 17 | 1461 | α-Humulene | 0.10 |
| 18 | 1487 | allo-aromadendrene | 0.18 |
| 19 | 1685 | α-Bisabolol | 0.35 |
| 20 | 1774 | α-Bisabolol oxide A | 0.11 |
| 21 | 1815 | Hexadecanal | 0.14 |
| 22 | 1870 | 1-Hexadecanol | 0.46 |
| 23 | 1879 | 1-Hexadecanol | 0.13 |
| 24 | 1894 | Rimuene | 0.28 |
| 25 | 1957 | Hexadecanoic acid | 0.68 |
| | | Total identified | 95.02 |
| | | Unknown | 4.98 |

Additional plants contemplated herein are of the genus Thymus.

The genus Thymus (/′taimes/TY-mes; thymes) contains about 350 species of aromatic perennial herbaceous plants and subshrubs to 40 cm tall in the family Lamiaceae, native to temperate regions in Europe, North Africa and Asia.

Stems tend to be narrow or even wiry; leaves are evergreen in most species, arranged in opposite pairs, oval, entire, and small, 4-20 mm long, and usually aromatic. Thyme flowers are in dense terminal heads with an uneven calyx, with the upper lip three-lobed, and are yellow, white, or purple.

Several members of the genus are cultivated as culinary herbs or ornamentals, when they are also called thyme after its best-known species, Thymus vulgaris or common thyme.

About 350 species, including:
Thymus adamovicii
Thymus altaicus
Thymus amurensis
Thymus boissieri
Thymus bracteosus
Thymus broussonetii
Thymus caespititius
Thymus camphoratus
Thymus capitatus
Thymus capitellatus
Thymus camphoratus
Thymus carnosus
Thymus cephalotus
Thymus cherlerioides
Thymus ciliatus
Thymus cilicicus
Thymus cimicinus
Thymus citriodorus (Thymus×citriodorus) syn. T. fragrantissimus, T. serpyllum citratus, T. serpyllum citriodorum. [7]-citrus thyme
Thymus comosus
Thymus comptus
Thymus curtus
Thymus decussatus
Thymus disjunctus
Thymus doerfleri
Thymus glabrescens
Thymus herba-barona
Thymus hirsutus
Thymus hyemalis
Thymus inaequalis
Thymus integer
Thymus lanuginosus, syn. T. serpyllum-woolly thyme
Thymus leucospermus
Thymus leucotrichus
Thymus longicaulis
Thymus longiflorus
Thymus mandschuricus
Thymus marschallianus
Thymus mastichina
Thymus membranaceus
Thymus mongolicus
Thymus moroderi
Thymus nervulosus
Thymus nummularis
Thymus odoratissimus
Thymus pallasianus
Thymus pallidus
Thymus pannonicus
Thymus praecox—creeping thyme
Thymus proximus
Thymus pseudolanuginosus, syn. T. serpyllum—woolly thyme
Thymus pulegioides-lemon thyme (8)
Thymus quinquecostatus
Thymus richardii
Thymus satureioides
Thymus serpyllum
Thymus sibthorpii
Thymus striatus
Thymus thracicus—lavender thyme
Thymus villosus
Thymus vulgaris—common thyme
Thymus zygis

List of plants that contain thymol include, but are not limited to:
Euphrasia rostkoviana
Lagoecia cuminoides
Monarda didyma
Monarda fistulosa
Mosla chinensis, Xiang Ru
Origanum compactum
Origanum dictamnus
Origanum onites
Origanum vulgare
Satureja thymbra
Thymus glandulosus
Thymus hyemalis
Thymus vulgaris
Thymus zygis
Trachyspermum ammi

Active ingredients in Thymus vulgaris:

| Chemical | Plant part | High ppm |
|---|---|---|
| 1-OCTEN-3-OL | Shoot | 150 |
| | Shoot | 65 |
| | Shoot | 80 |
| 2,5-DIETHYL-TETRAHYDROFURAN | Shoot | 0 |
| | Shoot | 6 |
| | Shoot | 6 |
| 3-OCTANOL | Shoot | 110 |
| | Shoot | 12 |
| | Shoot | 30 |
| ALPHA-GUAIENE | Shoot | 0.1 |
| | Shoot | 6 |
| | Shoot | 0 |
| ALPHA-HUMULENE | Shoot | 45 |
| | Shoot | 20 |
| | Shoot | 55 |
| ALPHA-PHELLANDRENE | Shoot | 0 |
| | Shoot | 40 |
| | Shoot | 12 |
| ALPHA-PINENE | Shoot | 0 |
| | Shoot | 265 |
| | Shoot | 325 |
| ALPHA-TERPINENE | Shoot | 840 |
| | Shoot | 990 |
| | Shoot | 990 |
| ALPHA-TERPINEOL | Shoot | 55 |
| | Shoot | 55 |
| | Shoot | 25 |
| ALPHA-THUJENE | Shoot | 320 |
| | Shoot | 0 |
| | Shoot | 0 |
| BETA-CARYOPHYLLENE | Shoot | 175 |
| | Shoot | 185 |
| | Shoot | 200 |
| BETA-GUAIENE | Shoot | 0.1 |
| | Shoot | 0 |
| | Shoot | 3 |
| | Shoot | 80 |
| BETA-PHELLANDRENE | Shoot | 60 |
| BETA-PHELLLANDRENE | Shoot | 70 |
| BETA-PINENE | Shoot | 30 |
| | Shoot | 30 |
| | Shoot | 560 |
| BORNEOL | Shoot | 55 |
| | Shoot | 30 |
| | Shoot | 15 |
| CAMPHENE | Shoot | 30 |
| | Shoot | 25 |
| | Shoot | 40 |
| CAMPHOR | Shoot | 0 |
| | Shoot | 0.1 |
| | Shoot | 0 |
| CARVACROL | Shoot | 1,285 |
| | Shoot | 24,850 |
| | Shoot | 23,765 |
| CARVONE | Shoot | 15 |
| | Shoot | 20 |
| | Shoot | 0.1 |
| CARYOPHYLLENE-OXIDE | Shoot | 75 |
| | Shoot | 55 |
| | Shoot | 45 |
| CIS-CARVEOL | Shoot | 0 |
| | Shoot | 0 |
| | Shoot | 3 |
| CIS-SABINENE-HYDRATE | Shoot | 20 |
| | Shoot | 0 |
| | Shoot | 55 |
| CITRONELLOL | Shoot | 12 |
| | Shoot | 0.1 |
| | Shoot | 0 |
| CITRONELLOL-BUTYRATE | Shoot | 0 |
| CITRONELLYL-BUTYRATE | Shoot | 0 |
| | Shoot | 15 |
| DIHYDROCARVONE | Shoot | 0 |
| | Shoot | 0 |
| | Shoot | 12 |
| EHTYL-CINNAMATE | Shoot | 0 |
| EO | Shoot | 31,000 |
| | Shoot | 31,000 |
| | Shoot | 31,000 |
| ETHYL-CINNAMATE | Shoot | 0 |
| | Shoot | 30 |
| GAMMA-TERPINENE | Shoot | 2,700 |
| | Shoot | 1,015 |
| | Shoot | 240 |
| GERANIOL | Shoot | 0 |
| | Shoot | 0 |
| | Shoot | 65 |
| GERANYL-ACETATE | Shoot | 0 |
| | Shoot | 0 |
| | Shoot | 15 |
| GERANYL-BUTYRATE | Shoot | 0 |
| | Shoot | 20 |
| | Shoot | 0 |
| GERANYL-HEXANOATE | Shoot | 0 |
| | Shoot | 0 |
| | Shoot | 6 |
| GERANYL-PROPIONATE | Shoot | 0 |
| | Shoot | 0 |
| | Shoot | 70 |
| GERMACRENE-D | Shoot | 0 |
| | Shoot | 0 |
| | Shoot | 50 |
| LIMONENE | Shoot | 110 |
| | Shoot | 55 |
| | Shoot | 90 |
| LINALOL | Shoot | 35 |
| | Shoot | 55 |
| | Shoot | 25 |
| METHYL-2-METHYL-BUTYRATE | Shoot | 6 |
| | Shoot | 12 |
| | Shoot | 9 |
| MYRCENE | Shoot | 750 |
| | Shoot | 565 |
| | Shoot | 0.1 |
| P-CYMENE | Shoot | 4,445 |
| | Shoot | 1,880 |
| | Shoot | 3,135 |
| TERPINEN-1-OL | Shoot | 6 |
| | Shoot | 15 |
| | Shoot | 0 |
| TERPINEN-4-OL | Shoot | 435 |
| | Shoot | 315 |
| | Shoot | 335 |
| TERPINOLENE | Shoot | 0 |
| | Shoot | 0.1 |
| | Shoot | 45 |
| THYMOL | Shoot | 18,560 |
| | Shoot | 385 |
| | Shoot | 280 |
| TRANS-BERGAMOTENE | Shoot | 9 |
| | Shoot | 9 |
| TRANS-BERGAMOTTENE | Shoot | 9 |
| TRANS-SABINENE-HYDRATE | Shoot | 25 |
| | Shoot | 120 |
| | Shoot | 0 |
| TRICYCLENE | Shoot | 0 |
| | Shoot | 0 |
| | Shoot | 3 |

Active ingredients on the EO of Thymus vulgaris according to some embodiments of the invention, include, but are not limited to:

| No. | RT (min) | Area % of total | Constituents* |
|---|---|---|---|
| 1 | 5.39 | 1.06 | alpha-Thujene |
| 2 | 5.63 | 1.07 | alpha-Pinene |
| 3 | 6.89 | 0.37 | beta-Pinene |
| 4 | 6.97 | 1.53 | beta-Myrcene |
| 5 | 7.53 | 0.33 | alpha-Phellandrene |
| 6 | 7.77 | 3.76 | Carene<δ-2-> |
| 7 | 8.04 | 0.29 | D-Limonene |
| 8 | 8.26 | 0.21 | beta-Phellandrene |
| 9 | 8.46 | 8.41 | para-Cymene |
| 10 | 8.96 | 30.90 | gamma-Terpinene |
| 11 | 9.48 | 0.47 | Terpineol |
| 12 | 12.55 | 0.46 | Terpinen-4-ol |
| 13 | 16.17 | 47.59 | Thymol |
| 14 | 17.32 | 2.68 | Caryophyllene |
| 15 | 19.03 | 0.78 | Cyclohexene, |
| | | | 1-methyl-4-(5-methyl-1- |
| | | | methylene-4-hexenyl) |
| Total | | 99.91% | |

Active ingredients of Satujera Thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil was dried using anhydrous magnesium sulfate and stored at 4° C. S. thymbra oil are analyzed by GC/MS. Nineteen compounds representing 98.8% of the oil sample are identified. The major components of Satureja thymbra L. oil are γ-terpinene (34.06%), carvacrol (23.07%) and thymol (18.82%). Also abundant are ρ-cymene (7.58%), caryophyllene (3.96%), α-terpinene (3.53%) and myrcene (1.70%).

Also contemplated herein are plants of the genus Satujera.

Satureja is a genus of aromatic plants of the family Lamiaceae, related to rosemary and thyme. It is native to North Africa, southern and southeastern Europe, the Middle East, and Central Asia. A few New World species were formerly included in Satureja, but they have all been moved to other genera. Several species are cultivated as culinary herbs called savory, and they have become established in the wild in a few places.

Examples include, but are not limited to:
Satureja adamovicii Šilic—Balkans
Satureja aintabensis P. H. Davis—Turkey
Satureja amani P. H. Davis—Turkey
Satureja atropatana Bunge—Iran
Satureja avromanica Maroofi—Iran
Satureja bachtiarica Bunge—Iran
Satureja boissieri Hausskn. ex Boiss.—Turkey, Iran
Satureja bzybica Woronow—Caucasus
Satureja x caroli-paui G. López—Spain (S. innota × S. montana)
Satureja cilicica P. H. Davis—Turkey
Satureja coerulea Janka—Bulgaria, Romania, Turkey
Satureja cuneifolia Ten—Spain, Italy, Greece, Albania, Yugoslavia, Iraq
Satureja x delpozoi Sánchez-Gómez, J. F. Jiménez & R. Morales—Spain (S. cuneifolia× S. intricata var. gracilis)
Satureja edmondii Briq.—Iran
Satureja× exspectata G. López—Spain (S. intricata var. gracilis × S. montana)
Satureja fukarekii Šilic—Yugoslavia
Satureja hellenica Halácsy—Greece
Satureja hortensis L.
Satureja horvatii Šilic-Greece, Yugoslavia
Satureja icarica P. H. Davis—Greek Islands
Satureja innota (Pau) Font Quer—Spain
Satureja intermedia C. A. Mey.—Iran, Caucasus
Satureja intricata Lange—Spain
Satureja isophylla Rech. f.—Iran
Satureja kallarica Jamzad—Iran
Satureja kermanshahensis Jamzad—Iran
Satureja khuzistanica Jamzad—Iran
Satureja kitaibelii Wierzb. ex Heuff.—Bulgaria, Romania, Yugoslavia
Satureja laxiflora K. Koch—Iran, Iraq, Turkey, Caucasus
Satureja linearifolia (Brullo & Furnari) Greuter—Cyrenaica region of Libya
Satureja macrantha C. A. Mey.—Iran, Iraq, Turkey, Caucasus
Satureja metastasiantha Rech.f.—Iraq
Satureja montana L.—winter savory—southern Europe, Turkey, Syria
Satureja mutica Fisch. & C. A. Mey.—Caucasus, Iran, Turkmenistan
Satureja nabateorum Danin & Hedge—Jordan
Satureja x orjenii Šilic—Yugoslavia (S. horvatii x S. montana)
Satureja pallaryi J. Thiébaut—Syria
Satureja parnassica Heldr. & Sart. ex Boiss.—Greece, Turkey
Satureja pilosa Velen.—Italy, Greece, Bulgaria
Satureja rumelica” Velen.—Bulgaria
Satureja sahendica Bornm.—Iran
Satureja salzmannii (Kuntze) P. W. Ball—Morocco, Spain
Satureja spicigera (K. Koch) Boiss.—Turkey, Iran, Caucasus
Satureja spinosa L.—Turkey, Greek Islands including Crete
Satureja subspicata Bartl. ex Vis.—Austria, Yugoslavia, Albania, Bulgaria, Italy
Satureja taurica Velen.—Crimea
Satureja thymbra L.—Libya, southeastern Europe from Sardinia to Turkey; Cyprus, Lebanon, Palestine
Satureja thymbrifolia Hedge & Feinbrun—Israel, Saudi Arabia
Satureja visianii Šilic.—Yugoslavia
Satureja wiedemanniana (Avé-Lall.) Velen.—Turkey

Active ingredients of Thymbra spicata:

| Compounds | % | RI¹ | RT² |
|---|---|---|---|
| α-pinene | 0.56 | 1028 | 3.64 |
| α-phellandrene | 0.64 | 1033 | 3.71 |
| camphene | 0.06 | 1073 | 4.36 |
| β-pinene | 0.10 | 1113 | 5.16 |
| δ-3-carene | 0.05 | 1155 | 6.10 |
| β-myrcene | 1.04 | 1170 | 6.51 |
| α-terpinene | 1.48 | 1184 | 6.90 |
| dl-limonene | 0.17 | 1202 | 7.43 |
| ß-phellandrene | 0.12 | 1212 | 7.69 |
| γ-terpinene | 10.73 | 1252 | 8.86 |
| p-cymene | 12.18 | 1276 | 9.69 |
| α-terpinolene | 0.05 | 1286 | 10.04 |
| oct-1-en-3-ol | 0.11 | 1454 | 16.17 |
| trans sabinene hydrate | 0.05 | 1465 | 16.59 |
| cis sabinene hydrate | 0.03 | 1547 | 19.73 |
| linalool | 0.03 | 1551 | 19.91 |
| trans caryophyllene | 1.28 | 1589 | 21.39 |
| 4-terpineol | 0.53 | 1598 | 21.79 |
| isoboreol | 0.21 | 1694 | 25.36 |
| d-carvone | 0.02 | 1728 | 26.55 |
| anethole | 0.04 | 1826 | 30.05 |
| caryophyllene oxide | 0.65 | 1968 | 34.87 |
| spathulenol | 0.15 | 2125 | 39.56 |
| thymol | 2.77 | 2218 | 41.80 |
| carvacrol | 66.86 | 2239 | 42.61 |
| naphthalene³ | 0.08 | 2281 | 44.26 |

¹RT—retention time;
²RI—retention index;
³naphthalene, 1,2,3,4,4a,5,6,7-octahydro-4a-methyl

Also contemplated herein are plants of the genus Thymbra.

Thymbra, common name Mediterranean thyme, is a genus of plants in the family Lamiaceae. As currently categorized, the genus has seven species and one subspecies. It is native to the Mediterranean region of southern Europe, North Africa, and the Middle East.

Examples include, but are not limited to:
Thymbra calostachya (Rech.f.) Rech.f.—Crete
Thymbra capitata (L.) Cav.—widespread from Morocco+Portugal to Turkey+Palestine
Thymbra sintenisii Bornm. & Azn.—Iraq, Turkey
Thymbra spicata L.—Greece, Turkey, Syria, Lebanon, Palestine, Israel, Iraq, Iran
Thymbra thymbrifolia (Hedge & Feinbrun) Bräuchler, comb. nov.—Israel, Palestine, Judean Desert, Khirbet el Mird
Thymbra nabateorum (Danin & Hedge) Brauchler, comb. nov.—W of Jordan and the adjacent N of Saudi Arabia
Thymbra linearifolia (Brullo & Furnari) Bräuchler, comb. nov.—Libya.
   Chemical Composition of Rhus coriaria (Sumac)

Characterization and identification of chemical compounds of Sumac using HPLC-MS method identified 191 compounds in Rhus coriaria and classified them as generally being:
78 hydrolysable tannins (e.g., gallotannins, e.g., penta, hexa, hepta, octa, nona and decagalloyl-glucoside)
59 flavonoids (e.g., Quercetin, Myrecetin 3-rhamnoside and Quercetin 3-glucoside)
9 anthocyanins (e.g., Delphidin-3-glucoside, Cyanidin 3-(2″-galloyl) galactoside, Cyanidin-3-glucoside, 7-methyl-cyanidin-3-(2″galloyl) galactoside, 7-methyl-cyanidin-3-galactoside)
2 isoflavonoids
2 terpenoids
1 diterpene
38 other unidentified compounds.

According to specific embodiments, the phenolic compounds in Sumac are the compounds that constitute its phytochemical activity along with anthocyanins. The most abundant phenolic compound in sumac fruits was found to be Gallic acid.

Hydrolysable tannins compose the highest percentage in the Sumac fruits, followed by flavonoids. This emphasizes the antioxidant potential of the fruit, a plant part contemplated herein as a specific embodiment. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Other active ingredients or any combinations thereof include, but are not limited to, methyla gallate, gathisflavone, sumaflavone, hinfikflavone, photocatechuic acid, penta-galloylglucose, hinokiflavone, β-caryophyllene, Delphidin-3-glucoside, Cyanidin 3-(2″-galloyl) galactoside, Cyanidin-3-glucoside, 7-methyl-cyanidin-3-(2″galloyl) galactoside, 7-methyl-cyanidin-3-galactoside, quercetin-3-glucoside, kampferol, myricetin, butein, D-limonine.

According to a specific embodiment, the active ingredient or combination thereof includes a volatile compound, e.g., terpene hydrocarbons, monoterpene and sesquiterpene hydrocarbons, specifically β-caryophyllene and α-pinene, Coririanaphthyl ether, Coriarioic acid and Coriariacthracenyl ester.

According to a specific embodiment, the active ingredient or combination thereof includes a fatty acid, e.g., oleic acid, linoleic acid, palmitic acid, β-caryophillene, cembrene stearic acid, Myristic acid, α-linolenic acid.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., thiamin B₁, riboflavin B₂, pyridoxine B₆, cyanocobalamin B₁₂, nicotinamide, biotin and ascorbic acid.

According to a specific embodiment, a methanol or ethanol extract is performed, e.g., ethanol concentration is 80%; extraction time is 1 h; extraction temperature is 40° C.; particle size 1.0 mm; and solvent to sumac ratios 15:1 ml/g. Other extraction procedures include, but are not limited to, those described in Sakhr and Khatib Heliyon. 2020 January; 6 (1): e03207, which is hereby incorporated by reference in its entirety.

According to another embodiment, the plant part is leaf.

Also contemplated herein are plants of the genus Rhus.

Examples include, but are not limited to:

### Asia and Southern Europe

Rhus chinensis Mill.—Chinese sumac
Rhus coriaria—Tanner's sumac
Rhus delavayi Franchet

### Australia, Pacific

Rhus taitensis Guill. (Northeast Australia, Malesia, Micronesia, French Polynesia)
Rhus sandwicensis A. Gray—neneleau (Hawaii)

### North America

Rhus aromatica—fragrant sumac
Rhus copallinum—winged or shining sumac
Rhus glabra—smooth sumac
Rhus integrifolia—lemonade sumac
Rhus kearneyi—Kearney sumac
Rhus lanceolata—prairie sumac
†Rhus malloryi Wolfe & Wehr—Ypresian, Washington
Rhus michauxii—Michaux's sumac
Rhus microphylla—desert sumac, littleleaf sumac
Rhus ovata—sugar sumac
tRhus republicensis Flynn, DeVore, & Pigg—Ypresian, Washington
tRhus rooseae Manchester—Middle Eocene, Oregon
Rhus trilobata Nutt.—skunkbush sumac
Rhus typhina—staghorn sumac
Rhus virens Lindh. ex A. Gray—evergreen sumac
   Chemical Composition of Panax ginseng (Ginseng)

Characterization and identification of chemical compounds of Ginseng using a variety of methods identified a large variety of compounds in Panax ginseng and classified them as generally being:
Saponin Glycosides (e.g., ginsenosides)
Phytosterols (e.g. stigmasterol, beta-sterol)
Sesquiterpenes (e.g. beta-alamene and beta-selinine)
Flavenoids (e.g. Kaempferol)
Polyacetylenes (e.g. panaxynol, ginsenoyne A)
Alkaloids (e.g. fumarine, girinimbin)
Polysaccharides
Phenolic compounds (e.g. elemicin, dauricin, maltol).

According to specific embodiments, the saponin compounds in Ginseng and the polysaccharide compounds are the compounds that constitute its phytochemical activity. The most abundant saponin compound in ginseng root was found to be ginsenoside. Polysaccharides from ginseng have been identified as NGP, WGP, 1-KGP, 4-KGP, WGPE and EGP, with WGP and WGPE being the most abundant, depending on the species of ginseng plant material used for extraction.

Most ginseng saponins belong to a family of steroids with a four trans-ring rigid steroid skeleton. They are also referred to as ginsenosides, triterpenoid saponins or dammarane derivatives. More than 200 saponins have been isolated from ginseng plants. In addition to ginseng root, saponins have been identified in ginseng leaves and stems, flower buds, fruits, berries, and seeds. Because steaming or heating changes the saponin profile of ginseng products, ginseng saponins have also been identified in the processed root, leaf, flower-bud and berry.

Ginseng saponins are divided into several groups. Two major groups are the protopanaxadiol (PPD)-type saponins with sugar moieties attached to the C-3 and/or C-20 and the protopanaxatriol (PPT) group with sugar moieties at C-6 and/or at C-20. Other groups include the ocotillol-type with a five-membered epoxy ring at C-20, the oleanane-type with a nonsteroidal structure, and the dammarane type with a modified C-20 side chain. As techniques are developed for chemical purification and structural identification, novel ginseng saponins continue to be discovered.

The table below shows ginsenoside compounds recovered from ginseng extracts prepared by different extraction procedures:

GINSENOSIDES

| | Solvent system^{a} | | Obtained | |
|---|---|---|---|---|
| Material | (volume ratio) | Detection | compound | Isolation efficiency^{c} |
| P. | Hex-n-BuOH-H₂O | TLC | Ginsenosides | 157, 13, 56, and 17 mg of Rb1, Re, |
| notoginseng, | (3:4:7) | | Rb1, Re, Rg1 | Rg1 and R1 from 283 mg MeOH |
| root | | | And | extract of five tablets |
| | | | notoginsenoside R1 | |
| P. ginseng, | CH₂Cl₂-MeOH-NH₄OAc-iPrOH | ELSD | Ginsenosides | 10.7, 11.0, 13.4 and 13.9 mg of Rf, |
| root | (6:2:4:3) | | Rf, Rd, Re, | Rd, Re and Rb1 from 480 mg |
| | | | and Rb1 | enriched fraction by macroporous resin |
| P. | CHCl₃-MeOH-2- | ELSD | Ginsenosides Rg1, | Not provided |
| notoginseng, | BuOH-H₂O (5:6:1:4) | | Rd, Rb1 and | |
| root | EtOAc-n-BuOH-H₂O | | notoginsenoside | |
| | (1:1:2) | | R1 | |
| Red P. | CH₂Cl₂-MeOH- | ELSD | Ginsenosides | 32.2, 26.6, 28.6 and 8.1 mg of Rg3, |
| ginseng, | H₂O-iPrOH | | Rg3, Rk1, | Rk1, Rg5 and F4 from 350 mg |
| steamed root | (6:6:4:1) | | Rg5 and F4 | enriched fraction by RP-C₁₈ |
| | | | | column |
| P. ginseng, | EtOAc-iPrOH-0.1% | UV | Ginsenoside Ro | 61 mg Ro from 100 mg enriched |
| root | formic acid H₂O (3:1:5) | | | sample by normal-phase MPLC |

^{a}Abbreviations: Hex: n-hexane; BuOH: butanol; CH₂Cl₂: methylene chloride; MeOH: methanol; NH₄OAc: ammonium acetate; iPrOH: isopropanol; CHCl₃: chloroform; EtOAc: ethyl acetate.
^{b}Abbreviations: TLC: thin layer chromatography; ELSD: evaporative light scattering detection; UV: ultraviolet.
^{c}Abbreviations: RP: reversed-phase; MPLC: medium-pressure liquid chromatography.

The table below shows the chemical formulae of 123 dammarane-type saponins isolated from various parts of Panax plants. They are placed in the order of the structure type.

Dammarane—type saponin ginsenosides

| No. | Name | Formula | Plant Material |
|---|---|---|---|
| 1 | Floralginsenoside M | C₅₃H₉₀O₂₂ | Flower buds of P. ginseng |
| 2 | Floralginsenoside N | C₅₃H₉₀O₂₂ | Flower buds of P. ginseng |
| 3 | Floralquinquenoside E | C₅₃H₉₀O₂₂ | Flower buds of P. quinquefolius |
| 4 | Ginsenoside Rh5 | C₃₇H₆₄O₉ | Roots and rhizomes of P. vietnamensis |
| 5 | Notoginsenoside FP1 | C₄₇H₈₀O₁₈ | Fruit pedicels of P. notoginseng |
| 6 | Notoginsenoside M | C₄₈H₈₂O₁₉ | Roots of P. notoginseng |
| 7 | Notoginsenoside N | C₄₈H₈₂O₁₉ | Roots of P. notoginseng |
| 8 | Notoginsenoside Rw1 | C₄₆H₇₈O₁₇ | Rhizomes of P. notoginseng |
| 9 | Notoginsenoside T3 | C₃₈H₆₆O₉ | Acid hydrolysate roots of P. notoginseng |
| 10 | Notoginsenoside U | C₄₂H₇₂O₁₄ | Roots of P. notoginseng |
| 11 | Quinquenoside L17 | C₄₇H₈₀O₁₈ | Leaves and stems of P. quinquefolius |
| 12 | Yesanchinoside D | C₄₄H₇₄O₁₅ | Underground part of P. japonicus |
| 13 | Yesanchinoside E | C₅₄H₉₂O₂₃ | Underground part of P. japonicus |
| 14 | Yesanchinoside F | C₅₆H₉₄O₂₄ | Underground part of P. japonicus |
| 15 | 20(S)-acetylated Rg2 | C₄₄H₇₄O₁₄ | Roots of P. quinquefolius |
| 16 | 20(R)-acetylated Rg2 | C₄₄H₇₄O₁₄ | Roots of P. quinquefolius |
| 17 | Malonylginsenoside Ra3 | C₆₂H₁₀₂O₃₀ | Fresh roots of P. ginseng |
| 18 | Malonylnotoginsenoside R4 | C₆₂H₁₀₂O₃₀ | Roots of P. ginseng |
| 19 | Notoginsenoside FP2 | C₅₈H₉₈O₂₆ | Fruit pedicels of P. notoginseng |
| 20 | Notoginsenoside FT1 | C₄₇H₈₀O₁₇ | Acid hydrolysate roots of P. notoginseng |
| 21 | Notoginsenoside L | C₅₃H₉₀O₂₂ | Roots of P. notoginseng |
| 22 | Notoginsenoside O | C₅₂H₈₈O₂₁ | Flower buds of P. notoginseng |
| 23 | Notoginsenoside P | C₅₂H₈₈O₂₁ | Flower buds of P. notoginseng |
| 24 | Notoginsenoside Q | C₆₃H₁₀₆O₃₀ | Flower buds of P. notoginseng |
| 25 | Notoginsenoside S | C₆₃H₁₀₆O₃₀ | Flower buds of P. notoginseng |
| 26 | Notoginsenoside T | C₆₄H₁₀₈O₃₁ | Flower buds of P. notoginseng |
| 27 | Quinquenoside L10 | C₄₇H₈₀O₁₇ | Leaves and stems of P. quinquefolius |
| 28 | Quinquenoside L14 | C₄₇H₈₀O₁₇ | Leaves and stems of P. quinquefolius |
| 29 | Yesanchinoside J | C₆₁H₁₀₂O₂₈ | Underground part of P. japonicus |
| 30 | Floralginsenoside A | C₄₂H₇₂O₁₆ | Flower buds of P. ginseng |
| 31 | Floralginsenoside C | C₄₁H₇₀O₁₅ | Flower buds of P. ginseng |
| 32 | Floralginsenoside H | C₅₀H₈₄O₂₁ | Flower buds of P. ginseng |
| 33 | Floralginsenoside J | C₄₈H₈₂O₂₀ | Flower buds of P. ginseng |
| 34 | Floralginsenoside Ka | C₃₆H₆₂O₁₁ | Flower buds of P. ginseng |
| 35 | Floralginsenoside Tc | C₅₃H₉₀O₂₄ | Flower buds of P. ginseng |
| 36 | Floralquinquenoside B | C₄₂H₇₂O₁₅ | Flower buds of P. quinquefolius |
| 37 | Floralquinquenoside D | C₄₂H₇₂O₁₅ | Flower buds of P. quinquefolius |
| 38 | Floranotoginsenoside B | C₅₃H₉₀O₂₄ | Flowers of P. notoginseng |
| 39 | Floranotoginsenoside C | C₅₃H₉₀O₂₄ | Flowers of P. notoginseng |
| 40 | Ginsenoside I | C₄₈H₈₂O₂₀ | Flower buds of P. ginseng |
| 41 | Ginsenoside II | C₄₈H₈₂O₂₀ | Flower buds of P. ginseng |
| 42 | Ginsenoside SL1 | C₃₆H₆₂O₁₁ | Steamed leaves of P. ginseng |
| 43 | Floralginsenoside B | C₄₂H₇₂O₁₆ | Flower buds of P. ginseng |
| 44 | Floralginsenoside D | C₄₁H₇₀O₁₅ | Flower buds of P. ginseng |
| 45 | Floralginsenoside E | C₄₂H₇₂O₁₅ | Flower buds of P. ginseng |
| 46 | Floralginsenoside F | C₄₂H₇₂O₁₅ | Flower buds of P. ginseng |
| 47 | Floralginsenoside G | C₅₀H₈₄O₂₁ | Flower buds of P. ginseng |
| 48 | Floralginsenoside I | C₄₈H₈₂O₂₀ | Flower buds of P. ginseng |
| 49 | Floralginsenoside K | C₄₈H₈₂O₂₁ | Flower buds of P. ginseng |
| 50 | Floralginsenoside O | C₅₃H₉₀O₂₄ | Flower buds of P. ginseng |
| 51 | Floralginsenoside P | C₅₃H₉₀O₂₃ | Flower buds of P. ginseng |
| 52 | Floralquinquenoside A | C₃₆H₆₂O₁₁ | Flower buds of P. quinquefolius |
| 53 | Floralquinquenoside C | C₄₂H₇₂O₁₅ | Flower buds of P. quinquefolius |
| 54 | Ginsenoside Rh6 | C₃₆H₆₂O₁₁ | Leaves of P. ginseng |
| 55 | Floralginsenoside La | C₄₈H₈₂O₁₉ | Flower buds of P. ginseng |
| 56 | Floralginsenoside Lb | C₄₈H₈₂O₁₉ | Flower buds of P. ginseng |
| 57 | Floranotoginsenoside D | C₅₃H₉₀O₂₃ | Flowers of P. notoginseng |
| 58 | Ginsenoside Rg7 | C₃₆H₆₀O₉ | Leaves of P. ginseng |
| 59 | Notopanaxoside A | C₃₆H₆₂O₁₀ | Roots of P. notoginseng |
| 60 | Notoginsenoside FT3 | C₄₇H₈₀O₁₈ | Acid hydrolysate roots of P. notoginseng |
| 61 | Floranotoginsenoside A | C₅₃H₉₀O₂₃ | Flowers of P. notoginseng |
| 62 | Ginsenoside ST2 | C₃₆H₆₂O₁₀ | Steamed leaves of P. ginseng |
| 63 | Notoginsenoside Rw2 | C₄₁H₇₀O₁₄ | Rhizomes of P. notoginseng |
| 64 | Notoginsenoside ST5 | C₄₇H₈₀O₁₈ | Steamed roots of P. notoginseng |
| 65 | Yesanchinoside H | C₅₃H₉₀O₂₃ | Underground part of P. japonicus |
| 66 | Ginsenoside Ki | C₃₆H₆₂O₁₀ | Leaves of P. ginseng |
| 67 | Ginsenoside Km | C₃₆H₆₂O₁₀ | Leaves of P. ginseng |
| 68 | Quinquenoside L2 | C₄₈H₈₂O₁₉ | Leaves and stems of P. quinquefolius |
| 69 | Dammar-25(26)-ene- | C₃₀H₅₂O₆ | Leaves of P. ginseng |
| | 3,6,12,20,22,24-hexanol | | |
| 70 | Floralginsenoside Kb | C₄₅H₇₆O₁₉ | Flower buds of P. ginseng |
| 71 | Floralginsenoside Kc | C₄₅H₇₆O₂₀ | Flower buds of P. ginseng |
| 72 | Floralginsenoside Ta | C₃₆H₆₂O₁₀ | Flower buds of P. ginseng |
| 73 | Vina-ginsenoside R25 | C₄₂H₇₀O₁₅ | Roots and rhizomes of P. vietnamensis |
| 74 | Floralginsenoside Tb | C₃₅H₆₂O₁₁ | Flower buds of P. ginseng |
| 75 | Quinquenoside L9 | C₄₂H₇₄O₁₅ | Leaves and stems of P. quinquefolius |
| 76 | Quinquenoside L16 | C₅₄H₉₄O₂₅ | Leaves and stems of P. quinquefolius |
| 77 | 25-OCH3-PPD | C₃₁H₅₆O₄ | Leaves of P. notoginseng |
| 78 | 25-OH-PPD | C₃₀H₅₄O₄ | Fruits of P. ginseng |
| 79 | 25-OH-PPT | C₃₀H₅₄O₅ | Fruits of P. ginseng |
| 80 | Notoginsenoside FT2 | C₄₇H₈₂O₁₈ | Acid hydrolysate roots of P. notoginseng |
| 81 | Notoginsenoside T4 | C₃₆H₆₂O₁₁ | Acid hydrolysate roots of P. notoginseng |
| 82 | Quinquenoside L1 | C₄₈H₈₀O₁₈ | Leaves and stems of P. quinquefolius |
| 83 | Quinquefoloside La | C₅₄H₉₂O₂₃ | Leaves of P. quinquefolius |
| 84 | Quinquefoloside Lc | C₅₄H₉₂O₂₃ | Leaves of P. quinquefolius |
| 85 | Dammar-(E)-20(22)- | C₃₀H₅₂O₃ | Acid hydrolysate roots of P. ginseng |
| | ene-3,12,25-triol | | |
| 86 | Notoginsenoside ST1 | C₃₆H₆₂O₁₀ | Steamed roots of P. notoginseng |
| 87 | Ginsenoside Rg6 | C₄₂H₇₀O₁₂ | Stem-leaves of P. ginseng |
| 88 | Ginsenoside Rs4 | C₄₂H₇₀O₁₂ | Steamed roots of P. notoginseng |
| 89 | Ginsenoside Rs6 | C₄₂H₇₀O₁₂ | Steamed roots of P. notoginseng |
| 90 | Isoginsenoside Rh3 | C₃₆H₆₀O₇ | Fruits of P. ginseng |
| 91 | Ginsenoside Rh5 | C₃₆H₆₀O₉ | Leaves of P. ginseng |
| 92 | Ginsenoside SL2 | C₄₂H₇₀O₁₄ | Steamed leaves of P. ginseng |
| 93 | Ginsenoside ST1 | C₃₆H₆₀O₁₀ | Steamed leaves of P. ginseng |
| 94 | Notoginsenoside ST2 | C₄₃H₇₄O₁₅ | Steamed roots of P. notoginseng |
| 95 | Notoginsenoside ST3 | C₄₃H₇₄O₁₅ | Steamed roots of P. notoginseng |
| 96 | Ginsenoside Rg8 | C₄₂H₇₀O₁₂ | Roots of P. quinquefolius |
| 97 | Notoginsenoside T1 | C₃₆H₆₀O₁₀ | Acid hydrolysate roots of P. notoginseng |
| 98 | Notoginsenoside T2 | C₃₆H₆₂O₁₀ | Acid hydrolysate roots of P. notoginseng |
| 99 | Ginsenoside Rg1-12,23- | C₄₂H₇₀O₁₄ | Leaves of P. ginseng |
| | epoxy | | |
| 100 | Ginsenoside Rh9 | C₃₆H₆₀O₉ | Leaves of P. ginseng |
| 101 | Quinquefoloside-Lb | C₅₃H₈₈O₂₂ | Leaves of P. quinquefolius |
| 102 | Ginsenoside Rk1 | C₄₂H₇₀O₁₂ | Processed roots of P. ginseng |
| 103 | Ginsenoside Rk2 | C₃₆H₆₀O₇ | Processed roots of P. ginseng |
| 104 | Ginsenoside Rk3 | C₃₆H₆₀O₈ | Processed roots of P. ginseng |
| 105 | Ginsenoside Rs5 | C₃₈H₆₂O₉ | Steamed roots of P. notoginseng |
| 106 | Ginsenoside Rs7 | C₃₈H₆₂O₉ | Steamed roots of P. notoginseng |
| 107 | Notoginsenoside T5 | C₄₁H₆₈O₁₂ | Acid hydrolysate roots of P. notoginseng |
| 108 | Ginsenoside Rz1 | C₄₂H₇₀O₁₂ | Steamed roots of P. notoginseng |
| 109 | Ginsenoside SL3 | C₄₂H₇₀O₁₄ | Steamed leaves of P. ginseng |
| 110 | Ginsenoside Rh8 | C₃₆H₆₀O₉ | Leaves of P. ginseng |
| 111 | Ginsenoside Rh7 | C₃₆H₆₀O₉ | Leaves of P. ginseng |
| 112 | Yesanchinoside G | C₅₃H₈₈O₂₃ | Underground part of P. japonicus |
| 113 | Yesanchinoside I | C₅₉H₁₀₀O₂₆ | Underground part of P. japonicus |
| 114 | Hexanordammaran | C₂₄H₄₀O₄ | Leaves of P. ginseng |
| 115 | Notoginsenoside R10 | C₃₀H₅₀O₉ | Steamed leaves of P. ginseng |
| 116 | Yesanchinoside A | C₄₄H₇₄O₁₆ | Underground part of P. japonicus |
| 117 | Yesanchinoside B | C₄₈H₈₂O₂₀ | Underground part of P. japonicus |
| 118 | Yesanchinoside C | C₄₇H₈₀O₁₉ | Underground part of P. japonicus |
| 119 | Panaxadione | C₃₀H₄₈O₅ | Seeds of P. ginseng |
| 120 | Polyacetyleneginsenoside | C₆₅H₁₀₀O₂₁ | Roots of P. ginseng |
| | Ro | | |
| 121 | Isodehydroprotopanaxatriol | C₃₀H₅₀O₃ | Acid hydrolysate roots of P. ginseng |
| 122 | 20,25-epoxy-dammaran- | C₃₀H₅₀O₃ | Acid hydrolysate roots of P. ginseng |
| | 2-en-6,12-diol | | |
| 123 | 3-methyl-28-nordammaran- | C₃₀H₅₀O₃ | Acid hydrolysate roots of P. ginseng |
| | 2-en-6,12-diol | | |

Analysis of ginseng root (Japanese ginseng) has indicated (per 100 grams root) 0.17 g (0.17%) total fat, 50 mg sodium, 8.82 g (8.82%) total carbohydrates comprising 2.3 g dietary fiber and 3.85 g sugars and 0.71 g (0.71%) protein content. calorimetric calculation showed that 100 g of ginseng root contains 37 kcal.

According to a specific embodiment, the active ingredient or combination thereof includes a ginsenoside, e.g. a protopanaxadiol (PPD)-type saponin with sugar moieties attached to the C-3 and/or C-20, a protopanaxatriol (PPT) saponin with sugar moieties at C-6 and/or at C-20, an ocotillol-type saponin with a five-membered epoxy ring at C-20, an oleanane-type saponin with a nonsteroidal structure, and a dammarane type saponin. Some specific ginsenosides include, but are not limited to notoginsenosides, yesanchinosides, panaxodione, floralginsenosides and ginsenosides Rg1, Rd, Re, Rb1, R1, Rg3, Rk1, Rf, Rg5, F4, Ro.

According to a specific embodiment, the active ingredient or combination thereof includes a volatile compound, e.g., terpene hydrocarbons, monoterpene and sesquiterpene hydrocarbons, specifically β-alamene and β-selenine.

According to a specific embodiment, the active ingredient or combination thereof includes a phytosterol, e.g., stigmasterol, beta-sterol.

According to a specific embodiment, the active ingredient or combination thereof includes a polyacetylene, e.g., panaxynol, ginsenoyne A.

According to a specific embodiment, the active ingredient or combination thereof includes a flavenoid, e.g., Kaempferol.

According to a specific embodiment, the active ingredient or combination thereof includes an alkaloid, e.g., fumarine, girinimbin.

According to a specific embodiment, the active ingredient or combination thereof includes a polysaccharide, e.g., WGP, KGP-1, KGP-4, WGPE, NGP, EGP.

According to a specific embodiment, the active ingredient or combination thereof includes a phenolic compound, e.g., elemicin, dauricin, maltol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 80%; extraction time is 24 h; extraction temperature is 80-90° C.; particle size 1.0 mm; and solvent to ginseng ratio of 20:1 ml/g. Other extraction procedures include, but are not limited to, those described in Dong et al. 2017 Phytother Res Aug; 19 (8): 684-688, which is hereby incorporated by reference in its entirety.

According to another embodiment, the plant part is leaf.

Also contemplated herein are plants of the genus Panax.

Examples include, but are not limited to:

| | Common name and |
|---|---|
| Ginseng Species | geographical designation |
| P. gensing | Korean ginseng |
| P. quinquefolius | American ginseng |
| P. notoginseng | Chinese ginseng |
| P. japonicas | Japanese ginseng |
| P. omiensis | Omei gensing |
| P. pseudoginseng | Himalayan ginseng |
| P. assamicus | N/A |
| P. shangianus | N/A |
| P. sinensis | N/A |
| P. stipuleanatus | Pingpien ginseng |
| P. trifolius | Dwarf ginseng |
| P. variabilis | N/A |
| P. vietnamensis | Vietnamese ginseng |
| P. wangianus | Narrow-leaved |
| P. bipinnatifidus | Feather-leaf bamboo ginseng |
| P. sokpayensis | N/A |
| P. zingiberensis | Ginger ginseng |

Korean ginseng cultivars suitable for use with the present invention include, but are not limited to: Chunpoong, Yunpoong, Gopoong, Sunpoong, Gumpoong, Cheongsun, Sunhyang, Sunun, Sunone, K-1, G-1 and Kowon. Chinese ginseng cultivars suitable for use with the present invention include, but are not limited to Jilin Huangguo Reshen, Jishen 01, Fuxing 01, Fuxing 02, Kangmei 01, Xinkaihe 01, Xinkaihe 02, Zhongnong Huangfengshen and Zhongda Linxiashen.

### Milk Thistle (Silybum Marianum L)

Milk thistle has two types: White and Blue. The main bioactive component of medicinal plant (milk thistle) is silymarin. Silymarin, which is a mixture of different flavonolignans such as, silybinin A and B (SBN A&B), isosilybinin A and B (ISBN A&B), silychristin (SCN), and silydianin (SDN).

Aziz et al. Food Sci Nutr. 2020 Nov. 9;9 (1): 244-250. doi: 10.1002/fsn3.1990. eCollection 2021 January, report the following biochemical profile for milk thistle: Mean Values for silymarin quantification (mg/g) by MAE (microwave assisted extraction) and soxhlet method in different M. T varieties as well as locations

| Locations | Varieties | MAE | Soxhlet |
|---|---|---|---|
| Islamabad | Blue | 1,840.6 ± 0.51 | 1669.5 ± 1.05 |
| | White | 1812.3 ± 1.30 | 1653.7 ± 1.71 |
| Faisalabad | Blue | 1804.9 ± 1.18 | 1644.9 ± 1.74 |
| | White | 1798.4 ± 1.27 | 1634.2 ± 1.14 |
| Jhang | Blue | 1792.9 ± 1.61 | 1624.7 ± 0.75 |
| | White | 1765.9 ± 1.50 | 1607.6 ± 0.99 |

Mean Values for antioxidant activity in extract of different M. T varieties as well as locations

| | Vari- | | | | |
|---|---|---|---|---|---|
| Locations | eties | TPC | TFC | DPPH | FRAP |
| Islamabad | Blue | 35.07 ± 1.75 | 29.09 ± 1.45 | 25.01 ± 1.25 | 17.69 ± |
| | | | | | 0.88 |
| | White | 32.60 ± 1.63 | 27.12 ± 1.35 | 24.93 ± 1.49 | 16.01 ± |
| | | | | | 0.80 |
| Faisalabad | Blue | 30.09 ± 1.50 | 22.09 ± 1.10 | 23.10 ± 1.15 | 15.03 ± |
| | | | | | 0.75 |
| | White | 28.65 ± 1.43 | 20.15 ± 1.00 | 22.01 ± 1.10 | 13.93 ± |
| | | | | | 0.55 |
| Jhang | Blue | 26.90 ± 1.34 | 17.61 ± 0.88 | 20.53 ± 0.82 | 11.90 ± |
| | | | | | 0.36 |
| | White | 24.17 ± 1.20 | 16.01 ± 0.80 | 18.9 ± 0.56 | 9.73 ± |
| | | | | | 0.29 |

TPC: total phenol content; TFC: total flavonoid content; DPPH: 2,2-Diphenyl-1-picrylhydrazyl assay; FRAP: ferric reducing antioxidant power assay

Anise (Pimpinella anisum)

The biochemical make up of anise as disclosed by Singh et al., “Chemical composition and antioxidant potential of essential oil and oleoresins from anise seeds (Pimpinella anisum L.)”, International Journal of Essential Oil Therapeutics (2008) 2, 122-130 is as follows:

TABLE 1
Chemical composition of Pimpinella anitum essential oil
analysed by GC/MS.

| compound | % MS | RI # | identification [symbol] |
|---|---|---|---|
| α-pinene | 0.1 | 927 | MS, RI, co-GC |
| sabinene | [symbol] | 964 | MS, RI, co-GC |
| myrcene | [symbol] | 983 | MS, RI, co-GC |
| α-phellandrene | [symbol] | 998 | MS, RI, co-GC |
| p-cymene | 0.1 | 1018 | MS, RI, co-GC |
| limonene | 0.8 | 1023 | MS, RI, co-GC |
| 1,8-cineole | 0.1 | 1026 | MS, RI, co-GC |
| [symbol] | [symbol] | 1029 | MS, RI, co-GC |
| fenchone | 5.0 | 1083 | MS, RI, co-GC |
| camphor | 0.2 | 1138 | MS, RI, co-GC |
| methyl [symbol] | 2.3 | 1192 | MS, RI |
| endo-fenchyl acetate | 0.1 | 1224 | MS, RI, co-GC |
| [symbol] | 0.5 | 1247 | MS, RI |
| [symbol] | 0.5 | 1253 | MS, RI |
| trans-anethole | 90.1 | 1294 | MS, RI |
| total | 99.9% | | |

Trace <0.05;
# the retention index (RI) was calculated using a homologous series of n-alkanes C₉-C₁₀; [symbol]
co-GC: co-injection with an authentic sample. Percentages are the mean of three runs and were obtained from electronic integration measurements using selective mass detector. [symbol]
indicates data missing or illegible when filed

TABLE 2
Chemical composition (%) of [symbol] resins (in
various solvents) of [symbol] analysed by GC/MS.

| compound | PS2 | PS3 | PS4 | PS5 | RI# | idendification [symbol] |
|---|---|---|---|---|---|---|
| [symbol] -cymene | — | — | t | — | 1028 | MS, RI, co-GC |
| [symbol] onene | t | — | 0.5 | t | 1031 | MS, RI, co-GC |
| [symbol] ,B- [symbol] | — | — | t | — | 1034 | MS, RI, co-GC |
| [symbol] -β- [symbol] | — | — | t | — | 1039 | MS, RI, co-GC |
| f [symbol] nchone | 1.2 | 0.3 | 0.9 | 0.4 | 1093 | MS, RI, co-GC |
| [symbol] | t | — | — | 0.3 | 1100 | MS, RI, co-GC |
| camphor | t | t | t | — | 1149 | MS, RI, co-GC |
| methyl chavicol | t | — | 0.5 | t | 1201 | MS, RI |
| endo- [symbol] acerate | t | t | t | t | 1226 | MS, RI |
| [symbol] -anisaldehyde | t | — | t | t | 1258 | MS, RI |
| trans-anethole | 14.1 | 5.7 | 19.7 | 6.4 | 1295 | MS, RI |
| [symbol] acid, | t | 0.3 | t | t | 1914 | MS, RI |
| methyl ester | | | | | | |
| [symbol] acid | 8.5 | 7.9 | 5.3 | 5.7 | 1968 | MS, RI, co-GC |
| [symbol] acid, | 1.2 | — | — | — | 1981 | MS, RI, co-GC |
| ethyl ester | | | | | | |
| linoleic acid, | t | 0.7 | 0.1 | t | 2080 | MS, RI, co-GC |
| methyl ester | | | | | | |
| oleic acid | t | 2.8 | 0.4 | 0.5 | 2086 | MS, RI, co-GC |
| methyl ester | | | | | | |
| oleic acid | 57.9 | 75.5 | 57.5 | 63.5 | 2130 | MS, RI, co-GC |
| oleic acid, ethyl | 11.0 | — | — | — | 2146 | MS, RI, co-GC |
| ester | | | | | | |
| stearic acid | 1.9 | — | — | — | 2157 | MS, RI, co-GC |
| 2- [symbol] ylglycerol | 2.3 | 2.8 | 2.1 | 3.6 | — | MS |
| squalene | — | t | 0.2 | t | — | MS |
| octacosan [symbol] l | t | t | 0.4 | 0.6 | — | MS |
| stigmasterol | t | t | 0.4 | 0.5 | — | MS |
| [symbol] | — | t | t | — | — | MS |
| total % | 98.1 | 96.0 | 88.0 | 81.5 | | |

PS2: ethanol extract;
PS3: methanol extract;
PS4: n-hexene extract;
PS5: petroleum benzene extract
Trace: <0.05;
# the retention index (RI) was calculated using a homologous series of n-alkanes C [symbol] -C [symbol] ; [symbol]
co-GC: co-infection with an authentic sample. [symbol] are the mean of three runs and were obtained from electronic integration measurements using a selective mass detector. [symbol]
indicates data missing or illegible when filed

Star Anise (Illicium verum)

Star anise is a woody species commonly known as ba jiao hui xiang in China. The major components of star anise are phenylpropanoids, flavonoids, neolignans, monoterpenoids and sesquiterpenoids. Star anise contains the following essential oid ocmpounds:

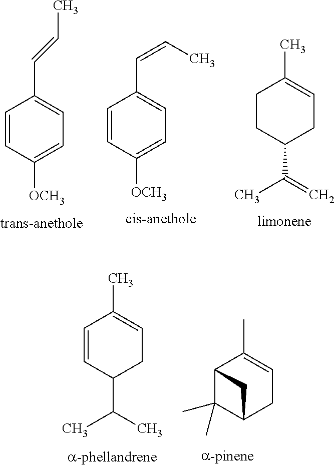

The biochemical makeup of star anise as disclosed by Sharafan et al., Illicium verum (Star Anise) and Trans-Anethole as Valuable Raw Materials for Medicinal and Cosmetic Applications, Molecules 2022, 27, 650, is as follows:

| Group of | Raw | |
|---|---|---|
| Compounds | Material | Compound Name |
| Phenolic | Essential oil | Trans-anethole, cis-anethole, |
| compounds | | estragole |
| | Fruit | Shikimic acid |
| | Root | Bliverin A, 4-allyl-2- |
| | | (3-methylbut-2-enyl)-1,6- |
| | | methylenedioxybenzene-3-ol, illicinole, |
| | | 3-hydroxy-4,5-me-thylenedioxyallyl- |
| | | benzene, (—)-illicinone-A, |
| | | 4-allyl-4-(3-methylbut-2-enyl)- |
| | | 1,2-methylenedioxycyclohexa- |
| | | 2,6-dien-5-one, 3,4-seco-(24Z)- |
| | | cycloart-4(28),24-diene-3,26-dioic |
| | | acid, 26-methyl ester |
| Monoterpenoids | Essential oil | α-Pinene, p-cymene, limonene, |
| | | linalool, terpinen-4-ol, |
| | | α-terpineol, eugenol, γ-terpineol, |
| | | o-3-canene, camphene, |
| | | β-myrcene, trans-ocymene, |
| | | terpinolene, γ-terpinene |
| Sesquiterpenoids | Essential oil | α-Phellandrene, α-muurolene, |
| | | β-caryophyllene, α-copaene, |
| | | trans-α-bergamotene, foeniculin, |
| | | β-elemene, cyperene, |
| | | α-caryophyllene, |
| | | (+)-9-epiledene, cubebene |
| | Root | Tashironin, tashironin A, |
| | | 11-O-debenzoyl-11α-O-2- |
| | | methylcyclopent-1- |
| | | enecarboxyltashironin, |
| | | veranisatins A-C |
| Flavonoids | Essential oil | Trans-chalcone |
| | Fruit | Kaempferol and glucosides, |
| | | quercetin and glucosides |
| Fatty acids | Fruit | Linoleic acid, stearic acid, |
| | | myristic acid |
| Alkylglucosides | Fruit | R-sec-butyl-D-glucopyranoside |
| Biphenyl-type | Leaf | Verimol G and verimol H, |
| neolignans | | 4,4′-dihydroxy-3,3′- |
| | | dimethoxy-9,9′-epoxylignan |
| Aldehydes | Essential oil | p-Anisaldehyde |
| Other | Essential oil | Anisoxide, 2-(1-cyclopentenyl)-furan, |
| | | isobornyl thiocyanoacetate |

### Curcumin

Curcumin is a bright yellow chemical produced by plants of the Curcuma longa species. It is the principal curcuminoid of turmeric (Curcuma longa), a member of the ginger family, Zingiberaceae. Curcumin may be extracted from tumeric and mango ginger. Chemically, curcumin is a diarylheptanoid, belonging to the group of curcuminoids, which are phenolic pigments responsible for the yellow color of turmeric. Curcumin incorporates a seven carbon linker and three major functional groups: an α, β-unsaturated β-diketone moiety and an aromatic O-methoxy-phenolic group. The aromatic phenols are connected by two α,β-unsaturated carbonyl groups. Curcumin is a diketone tautomer, existing in enolic form in organic solvents and in keto form in water. The diketones form stable enols and are readily deprotonated to form enolates; the α,β-unsaturated carbonyl group is a good Michael acceptor and undergoes nucleophilic addition. Curcumin has the following structure.

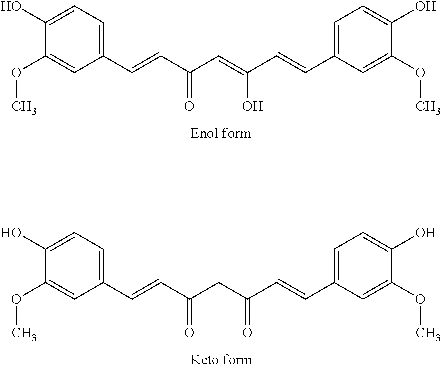 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

Chemical Composition of Boswellia species (Frankincense, Olibanum)

Olibanum, also known as frankincense, is a natural oleo-gum-resin that exudes from tappings in the bark of Boswellia trees. There are approximately 23 species of trees in the genus Boswellia, which grow mainly in Arabia, on the eastern coast of Africa and in India. Characterization and identification of chemical compounds of Olibanum using a variety of methods identified a large variety of compounds in the gum resin of Boswellia tree species and classified them as generally being:
Alcohol-soluble resins (e.g. diterpenes, triterpenes)
Highly aromatic essential oils (e.g. mono- and sesquiterpenes)
Water soluble gums

According to specific embodiments, Olibanum comprises 65-85% alcohol-soluble resins, about 5-9% highly aromatic essential oils and the remainder water soluble gums.

In India, the main commercial sources of Boswellia serrata are Andhra Pradesh, Gujarat, Madhya Pradesh, Jharkhand and Chhattisgarh. Regionally, it is also known by different names. The botanical origin and vernacular names of Boswellia serrata are given in below Table 1. Salai, an oleo gum-resin, is a plant exudate of genus Boswellia (Family: Burseraceae). It is tapped from the incision made on the trunk of the tree, which is then stored in specially made bamboo basket. The semi-solid gum-resin is allowed to remain in the basket for about a month during which its fluid content locally known as ‘ras’ keeps flowing out. The residue, semi-solid to solid part, is the gum-resin which hardens slowly into amorphous, tear-shaped products with an aromatic scent. Then, it is broken into small pieces by wooden mallet or chopper and during this process all impurities including bark pieces etc. are removed manually. The gum-resin is then graded according to its flavour, colour, shape and size. Generally four grades i.e. Superfine, Grade I, Grade II and Grade III are available in the market. The fresh gum obtained from the tree is hot with pleasant flavour and slightly bitter in taste. It had been the ‘frankincense’ of ancient Egyptians, Greeks and Romans who used it as prized incense, fumigant as well as a multipurpose aromatic. It is generally used in making incense powder and sticks.

TABLE 1
BOTANICAL ORIGIN AND VERNACULAR NAMES OF BOSWELLIA
SERRATA

| Botanical origin | Vernacular names |
|---|---|
| Division: Spermatophyta | English: Indian [symbol] or Indian frankincense |
| Sub-division: Angiospermae | Hindi: [symbol] |
| Tribe: Rosopsida | Bengali: [symbol] |
| Sub tribe: Rosidae S. lat. | Gujarati: Dhup, Gugali |
| Over-class: [symbol] | Kannada: Chitta, [symbol] |
| Class: Anacardiales | Malaysian: Parangi, Saambraani |
| Family: [symbol] | Tamil: Parangi, Saambraani |
| Genus: Boswellia | Telugu:Phirangi, Saambraani |
| Species: serrata | Sanskrit: [symbol] , Kundara, Shallaki. |

[symbol]

indicates data missing or illegible when filed

The oleo gum-resins contain 30-60% resin, 5-10% essential oils, which are soluble in the organic solvents, and the rest is made up of polysaccharides (˜65% arabinose, galactose, xylose) which are soluble in water. The resins have a fragrant aroma because of the presence of essential oils, and this accounts for their commercial importance.

According to specific embodiments, the common components of Olibanum belonging to the terpene and sesquiterpene families, or their terpenoid derivatives include, but are not limited to α- and β-pinene, α-limonene, myrcene, linalool, α-cubebene, γ-cadinene, β-bourbonene, and α-phellandrene dimer compounds in Olibanum are the compounds that constitute its phytochemical activity. Several oxygenated isoprenoid derivatives have also been identified, such as carbonyl derivatives (e.g., carvone, fenchone) and alcohol-containing terpene and sesquiterpene derivatives (e.g., transpinocarveol, cis-verbenol, and cembrenol), as well as ester-containing compounds (e.g., α-terpinyl acetate and bornyl acetate).

Diverse investigators have reported that limonene is the most abundant volatile in Olibanum, while others have identified octanol acetate, α-pinene and α-thujene as most abundant depending on the species of Boswellia plant material used for extraction.

More than 300 essential oils have been isolated from Boswellia ssp.

The table below shows the essential oils recovered from Olibanum extracts prepared by different extraction procedures, from diverse Boswellia ssp.:

| Number | Compound |
|---|---|
| 1 | 5,5-Dimethyl-1-vinylbicyclo-[2.1.1]-hexane |
| 2 | Anethol |
| 3 | Benzyl tiglate |
| 4 | trans-α-Bergamotene |
| 5 | Bornyl acetate |
| 6 | β-Bourbonene |
| 7 | Cadinene |
| 8 | γ-Cadinene |
| 9 | Camphene |
| 10 | Camphor |
| 11 | m-Camphorene |
| 12 | p-Camphorene |
| 13 | Carene-3 |
| 14 | (E)-β-Caryophyllene |
| 15 | Cembrene A |
| 16 | Cembrenol |
| 17 | 1,8 Cineol |
| 18 | Citronellol |
| 19 | α-Copaene |
| 20 | β-Copaene |
| 21 | p-Cymene |
| 22 | m-Cymene |
| 23 | Elemol |
| 24 | Elemicine |
| 25 | epi-Cubenol |
| 26 | Estragol |
| 27 | Eudesmol |
| 28 | 10-epi-γ-Eudesmol |
| 29 | Fenchone |
| 30 | Geraniol |
| 31 | Germacrene D |
| 32 | Humulene epoxide |
| 33 | Isoincensole |
| 34 | Isomenthone |
| 35 | Kessane |
| 36 | Limonene |
| 37 | Linalool |
| 38 | Linalyl acetate |
| 39 | Menthone |
| 40 | Methylchavicol |
| 41 | Methylisoeugenol |
| 42 | Methyleugenol |
| 43 | γ-Muurolene |
| 44 | Myrcene |
| 45 | Neocembrene A |
| 46 | Nerolidol |
| 47 | cis-β-ocimene |
| 48 | (Z)-Ocimene |
| 49 | (E)-β-Ocimene |
| 50 | Perillene |
| 51 | α-Phellandrene |
| 52 | β-Phellandrene |
| 53 | α-Pinene |
| 54 | β-Pinene |
| 55 | trans-Pinocarveol |
| 56 | Sabinene |
| 57 | cis-Sabinol |
| 58 | Terpinin-4-ol |
| 59 | Terpinen-4-ol |
| 60 | Terpinolene |
| 61 | α-Terpineol |
| 62 | α-Terpinene |
| 63 | α-Terpinene |
| 64 | γ-Terpinene |
| 65 | Terpinyl acetate |
| 66 | Terpinyl isobutyrate |
| 67 | Tetrahydrolinalool |
| 68 | α-Thujene |
| 69 | α-Thujone |
| 70 | β-Thujone |
| 71 | Tricyclene |
| 72 | Undecenol |
| 73 | trans-Verbenol |
| 74 | β-Ylangene |
| 75 | Zingiberene |
| 76 | Abieta-8, 12-diene |
| 77 | α-Amorphene |
| 78 | alloaromadendrene |
| 79 | Benzyl benzoate |
| 80 | Beyerene |
| 81 | Bisabolene |
| 82 | Isopentyl-2-methylbutanoate |
| 83 | cis-Calamenene |
| 84 | α-Cadinene |
| 85 | τ-Cadinol |
| 86 | 2-Carene |
| 87 | Campholenealdehyde |
| 88 | Caryophyllene oxide |
| 89 | cis-Carveol |
| 90 | (+) trans-Carveol |
| 91 | Carvone |
| 92 | α-Cedrene |
| 93 | Cedrol |
| 94 | Cembra-1,3,7,11-tetraene |
| 95 | Cembra-3,7,11,15-tetraene |
| 96 | Cembrene |
| 97 | Cembrene C |
| 98 | Citronellyl acetate |
| 99 | α-Cubebene |
| 100 | β-Cubebene |
| 101 | o-Cymene |
| 102 | Chrysanthenone |
| 103 | 1,4-Cyclohexadiene |
| 104 | p-Cymen-8-ol |
| 105 | Decanol |
| 106 | Decyl acetate |
| 107 | 2,6-Dimethoxytoluene |
| 108 | 3,5-Dimethoxytoluene |
| 109 | Duva-3,9,13-trien-1,5α-diol |
| 110 | Duva-4,8, 13-trien-1a,3α-diol |
| 111 | Duva-3,9, 13-trien-1,5α-diol-1-acetate |
| 112 | Duva-3,9, 13-triene-1α-ol-5,8-oxide-1-acetate |
| 113 | β-Elemene |
| 114 | Farnesyl acetate |
| 115 | Geranyl acetate |
| 116 | α-Gurjunene |
| 117 | Hedycariol |
| 118 | 1,3,6-Trimethylencycloheptane |
| 119 | 1-Hexanol |
| 120 | Hexyl acetate |
| 121 | Hexyl hexanoate |
| 122 | α-Humulene |
| 123 | Incensole |
| 124 | Incensole acetate |
| 125 | Isodurene |
| 126 | Isocembrene |
| 127 | Isophyllocladene (kaur-15-ene) |
| 128 | Kaurene |
| 129 | Ledol |
| 130 | Maaliane |
| 131 | p-Mentha-1,5-dien-8-ol |
| 132 | o-Methyl anisole |
| 133 | α-Muurolene |
| 134 | α-Muurolol |
| 135 | Myrtenal |
| 136 | Naphthalene |
| 137 | Naphthalene 1,2,3,4,4a,7-hexahydro- |
| | 1,6-dimethyl-4-(1-methylethyl |
| 138 | Neryl acetate |
| 139 | cis-Nerolidol |
| 140 | (S)-trans-Nerolidol |
| 141 | (E)-Nerolidol |
| 142 | 1-Octanol |
| 143 | n-Octanol |
| 144 | Octanol acetate |
| 145 | Octyl acetate |
| 146 | Octyl formate |
| 147 | allo-Ocimene |
| 148 | Phenanthrene-7-ethenyl-9, 10, 10a- |
| | dodeca-hydro-1-1-4a- |
| | 7-tetramethyl |
| 149 | α-Phellandrene epoxide |
| 150 | Phyllocladene |
| 151 | α-Pinene-epoxide |
| 152 | 1-6-Pinene |
| 153 | 2-6-Pinene |
| 154 | Isopinocampheol |
| 155 | Piperitone |
| 156 | Pyrimidine |
| 157 | Sabinyl acetate |
| 158 | Sandaracopimara-8(14)-15-diene |
| 159 | Sclarene |
| 160 | α-Selinene |
| 161 | β-Selinene |
| 162 | δ-Selinene |
| 163 | trans-Terpine |
| 164 | 4-Terpineol |
| 165 | Terpinolene |
| 166 | Isoterpinolene |
| 167 | 2,4(10)-Thujadiene |
| 168 | Thujopsene |
| 169 | Thunbergol |
| 170 | Isomyl-valerate |
| 171 | Verticilla-4(20),7,11-triene |
| 172 | Verbenone |
| 173 | cis-Verbenol |
| 174 | Verticiol |
| 175 | Viridiflorol |
| 176 | Benzene, 1methoxγ-2-methyl |
| 177 | endo-Borneol |
| 178 | γ-Campholene aldehyde |
| 179 | α-Campholene aldehyde |
| 180 | Cara-2,4-diene |
| 181 | Carvacrol |
| 182 | Carvotanacetone |
| 183 | trans-Dihydrocarvone |
| 184 | Cumin alcohol |
| 185 | m-Cymene-8-ol |
| 186 | p-Cymene-9-ol |
| 187 | p-Cymenene |
| 188 | Dodecanol |
| 189 | Eucalyptol |
| 190 | Eucarvone |
| 191 | Isopropyl benzaldehyde |
| 192 | Isopropyl benzalcohol |
| 193 | cis-1,2-Limonene epoxide |
| 194 | 8,9-Limonene epoxide II |
| 195 | 8,9-Limonene-epoxide I |
| 196 | trans-1,2-Limonene epoxide |
| 197 | cis-Linalool oxide |
| 198 | trans-Linalool oxide |
| 199 | p-Mentha-1,5-diene-7-ol |
| 200 | p-Mentha-1,8-diene-4-ol |
| 201 | cis-p-Menth-2-en-1-ol |
| 202 | cis-p-Mentha-1(7),8-diene-2-ol |
| 203 | cis-p-Mentha-2,8-diene-1-ol |
| 204 | trans-p-Menth-2-en-1-ol |
| 205 | trans-p-Mentha-1(7),8-diene-2-ol |
| 206 | trans-p-Mentha-2,8-diene-1-ol |
| 207 | 2,4(8)-p-Menthadiene |
| 208 | p-Mentha-6,8-dien-2-one |
| 209 | p-Methylanisole |
| 210 | Myrtenol |
| 211 | Nerol |
| 212 | trans-Ocimene |
| 213 | (E)-β-Ocimene epoxide |
| 214 | α-Phellandrene-dimer |
| 215 | α-Phellandrene-8-ol |
| 216 | α-Pinene oxide |
| 217 | Pinocamphone |
| 218 | Pinocarvone |
| 219 | Piperitenone |
| 220 | Isopiperitenone |
| 221 | trans-Piperitol |
| 222 | α-Terpineol |
| 223 | Sabina ketone |
| 224 | cis-Sabinene hydrate |
| 225 | trans-Sabinene hydrate |
| 226 | trans-Sabinol |
| 227 | 2,5-Dimethylstyrene |
| 228 | cis-1,2-Epoxγ-terpin-4-ol |
| 229 | Thuj-3-en-10-al |
| 230 | Thujanol |
| 231 | Thunbergene |
| 232 | Thymol |
| 233 | Umbellulone |
| 234 | Verticellol |
| 235 | 5,5-Dimethyl-1-vinylbicyclo-[2.1.1]-hexane |
| 236 | p-Anisaldehyde |
| 237 | Aromadendrene |
| 238 | Benzyl tigilate |
| 239 | p-Camphorene |
| 240 | Isocaryophyllene |
| 241 | Cumaldehyde |
| 242 | Cyclosativene |
| 243 | γ-Eudesmol |
| 244 | Guaioxide |
| 245 | 5-Guaiene-11-ol |
| 246 | Isogermacrene D |
| 247 | 4-Methylene-1-(1-methylethyl)-bicyclo[3.1.0]hex-2-ene |
| 248 | -Methyl-5-(1-methylethyl)-1,3-cyclohexadiene monoepoxide |
| 249 | n-Pentadecan |
| 250 | Perilla alcohol |
| 251 | Perillol |
| 252 | Thujol |
| 253 | m-Thymol |
| 254 | α-Ylangene |
| 255 | γ-Campholene aldehyde |
| 256 | n-Decanoic acid |
| 257 | β-Eudesmene |
| 258 | β-Cyclogeranylacetate |
| 259 | n-Hexanoic acid |
| 260 | Hexylcaprylate |
| 261 | Incensyl acetate |
| 262 | Incensole oxide |
| 263 | Incensole oxide acetate |
| 264 | Lauric acid |
| 265 | p-Methylacetophenone |
| 266 | p-Methyleugenol |
| 267 | β-Myrcene |
| 268 | n-Nonanoic acid |
| 269 | n-Octanoic acid |
| 270 | 3,4-Dimethoxystyrene |
| 271 | α-Cadinol |
| 272 | 1,Hydroxγ-1,7-dimethyl-4-isopropyl-2,7- |
| | cyclodecadiene |
| 273 | 1,5,5,8-Tetramethyl-12-oxabicyclo-[9.1.0]- |
| | dodeca-3,7-diene |
| 274 | 1-Methyl-4-(1-methylethenyl)-1,2-cyclohexanediol |
| 275 | trans-p-Mentha-2,8-dienol |
| 276 | 1,2,3,4,6,8a-hexahydro-1-isopropyl-4,7-dimethyl- |
| | naphthalene |
| 277 | 2-Isopropenyl-4a,8-dimethyl-1,2,3,4,4a,5,6,8a- |
| | ctahydronaphthalene |
| 278 | 3,5-Dimethoxytoluene |
| 279 | (Z)-α-Hydroxymanool |
| 280 | Hydroxγ-manool |
| 281 | Methyl linoleate |
| 282 | 1-Acetyl-4-isopropenylcyclopentene |
| 283 | 2,4-Dimethylacetophenone |
| 284 | α-Amyrenone |
| 285 | β-Amyrenone |
| 286 | 10-Hydroxγ-4-cadinen-3-one |
| 287 | 2-Hydroxγ-1,4-cineole |
| 288 | Cryptone |
| 289 | Eucarvone |
| 290 | Isopropylidencyclohexane |
| 291 | 1,2,4-Trihydroxγ-p-menthane |
| 292 | Δ⁴-p-Menthen-2-one |
| 293 | 5-Hydroxγ-p-menth-6-en-2-one |
| 294 | Myrtenoic acid |
| 295 | Nopinone |
| 296 | 3,6,6,-Trimethyl-norpinan-2-one |
| 297 | o-Methylacetophenone |
| 298 | Perillaaldehyde |
| 299 | Phellandra |
| 300 | Pinocamphone/isopinocamphone |
| 301 | Thujone |
| 302 | 24-Noroleana-3, 12-diene |
| 303 | 24-Noroleana-3,9(11), 12-triene |
| 304 | 24-Norursa-3, 12-diene |
| 305 | 24-Norursa-3,9(11), 12-triene |
| 306 | 24-Norursa-3.12-dien-11-one |
| 307 | α-Amyrine |
| 308 | epi-α-Amyrine |
| 309 | β-Amyrine |
| 310 | Lupeol |
| 311 | Terpinenyl acetate |
| 312 | 1,5-Isopropyl-2-methylbicyclo[3.1.0]hex-3-en-2-ol |
| 313 | α-Campholenal |
| 314 | (3E,5E)-2,6-Dimethyl-1,3,5,7-octatetraene |
| 315 | (E)-2,3-Epoxycarene |
| 316 | 3,4-Dimethylstyrene |
| 317 | 1-(2,4-Dimethylphenyl)ethanol |
| 318 | 4-Methylbenzoic acid |
| 319 | p-Menth-1(7)-en-2-one |
| 320 | Caryophyllene |
| 321 | Methylcycloundecanecarboxylate |
| 322 | Nonanoic acid |
| 323 | Hexadecanoic acid |
| 324 | 1,4-Cineol |
| 325 | Sabinene hydrate |
| 326 | Methyl-trans-2-cis-4-decadienoate |
| 327 | 2-Hydroxγ-5-methoxγ-acetophenone |
| 328 | (E)-β-Farnesene |
| 329 | 2-Dodecenoic acid methyl ester |
| 330 | Calacorene |
| 331 | n-Dodecanoic acid |
| 332 | α-Guaiol |
| 333 | Caryophylla-3(15), 7(14)-dien-6-ol |
| 334 | Cadalene |
| 335 | Eudesma-4(15),7-dien-1β-ol |
| 336 | n-Heptadecane |
| 337 | n-Tetradecanoic acid |
| 338 | n-Octadecane |
| 339 | Galaxolide |
| 340 | Manool |

Although many Boswellia species produce Olibanum, the major sources of commercial Olibanum are B. sorrata (India), B. sacra (Oman), and B carteri (Somalia). The table below shows the major components of Olibanum derived from diverse Boswolia species, according to their percentage representation:

| Boswellia | | Predominant | Percentage |
|---|---|---|---|
| specie | Source of resin | compound(s) | (%) |
| B. serrata | Commercial | Myrcene | 38 |
| | (Hamburg, Germany) | | |
| B. serrata | NA | α-Thujene | 22.7-47.4 |
| B. serrata | NA | α-Thujene | 29.3 |
| B. serrata | NA | α-Thujene | 61.36 |
| B. carteri | Purchased from the local | Duva-3,9, 13-trier- | 21.4 |
| | market of herbs | 1a-ol-5,8-oxide- | |
| | and spices in Egypt | 1-aceta [symbol] | |
| B. sacra | Botanically certified | E-β-Ocimene | 32.3 |
| | oleogum resin | | |
| B. carteri/ | NM | Octanol acetate | 45.2 |
| sacra | | | |
| B. carteri | Authentic sample | Octyl acetate | 39.3 |
| | from Ethiopia certi [symbol] | | |
| | for its authenticity | | |
| | from the Agriculture | | |
| | Department of the | | |
| | Ethiopian government | | |
| B. rivae | NA | Limonene | 28.0 |
| B. rivae | Authentic sample | α-Pinene | 16.7 |
| | from Ethiopia | | |
| B. rivae | NA | α-Pinene | 13.3 |
| B. rivae | NA | Octanol | 17.8 |
| B. neglecta | NA | α-Pinene | 16.7 |
| B. neglecta | Authentic sample | α-Pinene | 21.3 |
| | from Ethiopia | | |
| B. papyrifer | NA | Octyl acetate | 63.5 |
| B. papyrifer | NA | Octyl acetate | 56.0 |
| B. pirottae | NA | Trans-Verbenol | 15.5 |
| B. pirottae | NA | Terpinen-4-ol | 14.6 |
| B. frereana | Commercial (Hamburg, | α-Pinene | 38.0 |
| | Germany) | | |

[symbol]

indicates data missing or illegible when filed

One exemplary analysis of Olibanum has indicated the following components
Acid resin (6%), soluble in alcohol and having the formula C20H3204
gum (similar to gum arabic) 30-36%
3-acetyl-beta-boswellic acid (Boswellia sacra)
alpha-boswellic acid (Boswellia sacra)
incensole acetate, C₂₁H₃₄O₃
phellandrene

Another analysis of B. serrata resin revealed that the resinous part of Boswellia serrata contains monoterpenes (a-thujene); diterpenes (macrocyclic diterpenoids such as incensole, incensole oxide, iso-incensole oxide, a diterpene alcohol [serratol]); triterpenes (such as α- and β-amyrins); pentacyclic triterpenic acids (boswellic acids); tetracyclic triterpenic acids (tirucall-8,24-dien-21-oic acids). The structures of four major pentacyclic triterpenic acids (boswellic acids) as also some of their characteristic features of four pentacyclic triterpene acids (Boswellic acid) are given in the following table:

| | | Acetyl-β-Boswellic | 11-keta-β-Boswellic | Acetyl-11-keto-β- |
|---|---|---|---|---|
| Properties | β-Boswellic acid | acid | acid | Boswellik acid |
| Molecular formula | [symbol] | [symbol] | [symbol] | [symbol] |
| Molecular weight | 456.7 | 408.74 | 470.69 | 542.73 |
| Chemical name | [symbol] | [symbol] | [symbol] | [symbol] |
| | [symbol] | [symbol] | [symbol] | [symbol] |
| Melting point | [symbol] | [symbol] | [symbol] | [symbol] |
| Specific rotation | [symbol] | [symbol] | [symbol] | [symbol] |
| UV-MeOH | Maxima at 208 nm | Maxima at 208 nm | Maxima at 250 nm | Maxima at 250 nm |
| HNR (in CDCl₃, δ ppm) | [symbol] | [symbol] | [symbol] | [symbol] |
| | [symbol] | [symbol] | [symbol] | [symbol] |
| | [symbol] | [symbol] | [symbol] | [symbol] |
| | [symbol] | [symbol] | [symbol] | [symbol] |
| | | [symbol] | [symbol] | |
| FTIR (in NBr, cm²) | [symbol] | [symbol] | [symbol] | [symbol] |
| | | | | [symbol] |
| | | | | [symbol] |
| GC-MS | [symbol] | [symbol] | [symbol] | [symbol] |
| | [symbol] | [symbol] | and 18 due to H₂O)); 232 | and 18 due to H₂O)); 232 |
| | [symbol] | [symbol] | (base peak); | 232 (base peak); Other |
| | [symbol] | | Other fragrances: 217, 179 | fragrances: 217, 175 |
| | | | 161, 135 | 161, 135 |

[symbol]

indicates data missing or illegible when filed

The Olibanum gum component contains polysaccharides and polymeric components. The proteoglycans in Olibanum comprise mainly D-galactose units in the main chain and glucuronic acid, uronic acids, 4-O-methyl-glucuronic acid and arabinose in the side chains.

According to a specific embodiment, the active ingredient or combination thereof includes an alcohol soluble acid resin, a water soluble gum, an alpha-boswellic acid, anincensole acetate and a phellandrene.

According to a specific embodiment, the active ingredient or combination thereof includes a volatile compound, e.g. a-Thujene, Duva-3,9,13-triene-1a-ol-5,8-oxide-1-acetate, E-6-Ocimene, Octanol acetate, Octyl acetate, Limonene, α-Pinene, Octanol, Trans-Verbenol and Terpinen-4-ol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, a water or alcohol extract is performed.

In some embodiments, the Olibanum is prepared by water extract. An exemplary water extract is described herein:

Preparation of olibanum extract by water. At first, Olibanum is carefully powdered. The powder (25 g) is mixed with 200 ml of deionized water and stirred with 800 rpm overnight at room temperature. This mixture is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at-20 C and then freeze-dried-58 C and 0.5 Torr for 24 h to yield 4.02 gr of water soluble extract. At the next step, the resulted powder is dissolved in 100 ml methanol and stirred for 12 hr. at room temperature, then allowed to settle. The precipitate phase is collected and dried in an oven. Again the powder is dissolved in deionized water, centrifuged repeatedly and refiltered. The filtrates can be stored and then freeze-dried.

In some embodiments, the Olibanum is prepared by alcohol extract. An exemplary alcohol extract is described herein:

Preparation of olibanum extract by alcohol: In this method, 100 gr of Olibanum powder with 400 ml of methanol is mixed. This mixture is then stirred at 650 rpm for 24 hours. The resulting mixture is made up of two phases, the upper phase is alcoholic and yellow, and contains substances that are soluble in alcohol. The material is then dried in an oven at 50 C. The bottom phase has a sedimentary and white state, which is set to in the oven until dry. The resulting powder in the water is well dissolved and the obtained solution is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at −20 C and then freeze-dried.

Other extraction procedures include, but are not limited to, those described in Mertens et al, et al. 2009, Flavor and Fragrance, 24:279-300 and Hamm et al, Phytochemistry 2005, 66:1499-1514, which are hereby incorporated by reference in their entirety.

Also contemplated herein are Olibarum and other compositions from trees of the genus Boswellia.

Examples include, but are not limited to:

Some Boswellia Species
B. socotrana
B. elongata
B. ameero
B. carteri
B. neglecta
B. sacra
B. thurifera
B. frereana
B. dioscorides
B. rivae
B. papyrifera
B. serrata

### Chemical Composition of Gynostemma Pentaphyllum (Jiaogulan)

Gynostemma pentaphyllum is a perennial herb from the Cucurbitaceae family, with 5-lobed leaves and a gourd-like, inedible fruit which grows in forests, thickets or roadsise on mountain slopes in many areas of Northeast and Southeast Asia, including China, Taiwan, S Korea, Japan, Thailand, Vietnam and Laos. G. pentphyllum also grows in Bangladesh, Bhutan, India, Indonesia, Malaysia, Myanmar, Nepal, New Guinea and Sri Lanka. Jiaogulan is prized for its reputation as a “longevity plant”. Characterization and identification of chemical compounds of Gynostemma pentaphyllum using a variety of methods identified a large variety of compounds in Gynostemma pentaphyllum (Thun.) Makino and classified them as generally being:
Saponin Glycosides (e.g., gypenosides)
Phenolic compounds
Flavenoids (e.g. Kaempferol, quercetin, rutin, ombuin, isorahmnetin)
Polysaccharides
Sterols (e.g. ergostane, cholestane, stigmastane)
Trace elements (e.g. Cu, Fe, Zn, Mn, Co, Ni, Se, Mo and Sr)
Carotenoids
Volatiles (e.g. malonic acid, benzyl-O-beta-D-glucopyranoside, lutein, vomifoliol, palmitic acid, linoleic acid)

According to specific embodiments, the saponin compounds in Jiaogulan and the polysaccharide compounds are the compounds that constitute its phytochemical activity. The most abundant saponin compound in Jiaogulan was found to be gypenoside.

Most Jiaogulan saponins belong to a family of triterpenoid saponins. They are also referred to as gypenosides, and dammarane derivatives. More than 150 saponins have been isolated from G. pentaphyllum plants. Saponins have been identified in Jiaogulan leaves and stems, flower buds, fruits, berries, and seeds.

The table below shows the phytochemical properties of 5 different Gynostemma pentaphyllum samples from different sources:

| | | TPC | TSC | TFC | RUTIN | QUERCITIN | R + Q |
|---|---|---|---|---|---|---|---|
| | | (mg | (mg | (mg | CONTENT | CONTENT | (umol |
| SAMPLE | SOLVENT | GAE/g) | GE/g) | RE/g) | (ug/g) | (ug/g) | QE/g) |
| GP1 | 50% aceto [symbol] | 44.3 | 38.02 | 21.44 | 3049.5 | 4906.5 | 21.2 |
| | 50% ethan [symbol] | 37.5 | 41.39 | 26.40 | 7948.2 | 7431.8 | 37.6 |
| | 100% etha [symbol] | 33.6 | 87.28 | 26.87 | 11235.4 | 7279.1 | 42.5 |
| GP2 | 50% aceto [symbol] | 14.9 | 90.17 | 10.6 | 2527.3 | 117.5 | 4.5 |
| | 50% etha [symbol] | 12.9 | 114.48 | 14.27 | 3588.1 | 136.2 | 6.3 |
| | 100% etha [symbol] | 6.9 | 132.57 | 13.84 | 2131.9 | 166.2 | 4.0 |
| GP3 | 50% aceto [symbol] | 12.3 | 47.62 | 10.52 | 8614.9 | 358.9 | 15.3 |
| | 50% ethan [symbol] | 10.6 | 59.13 | 9.51 | 9954.0 | 411.0 | 17.7 |
| | 100% etha [symbol] | 6.7 | 64.57 | 8.05 | 7193.0 | 549.4 | 13.6 |
| GP4 | 50% aceto [symbol] | 43.2 | 77.64 | 63.48 | 1409.2 | 241.3 | 3.1 |
| | 50% ethan [symbol] | 30.4 | 82.12 | 54.04 | 680.2 | 150.8 | 1.6 |
| | 100% etha [symbol] | 17.7 | 104.1 | 36.47 | 579.4 | 151.3 | 1.4 |
| GP5 | 50% aceto [symbol] | 13.1 | 23.61 | 14.55 | nd | nd | |
| | 50% ethan [symbol] | 10.2 | 60.7 | 16.53 | nd | nd | |
| | 100% etha [symbol] | 8.9 | 123.97 | 22.11 | nd | nd | |

[symbol]

indicates data missing or illegible when filed

GP1-5 represents G. pentaphyllum samples from different sources. Data are per gram of dry botanical basis and are expressed as mean (SD. Different letters represent significant differences (P<0.05). nd stands for not detectable. TPC, TSC, and TFC stand for total phenolic content, total saponin content, and total flavonoid content by spectrometric methods, respectively. GAE, GE, RE, and QE stand for gallic acid equivalents, gypenoside equivalents, rutin equivalents, and quercetin equivalents. Rutin and quercetin contents were flavonoid profile obtained by HPLC. R+Q stands for total amount of rutin and quercetin.

Ethanol extraction: 12 g sample in 250 ml 100% ethanol, 5 hours in Soxhlet apparatus.

50% acetone extraction and 75% ethanol extraction: 2 g sample in 20 ml solvent at ambient temperature and filtration through 45 micron filter.

Water content of the Jiaogulan samples ranged from 3.79 to 7.57 g/100 g sample. Dietary fiber content ranged from 0.6 g/g to 0.24 g/g sample. Selenium content ranged from 1.7 mg/kg to 0.94 mg/kg.

According to a specific embodiment, the active ingredient or combination thereof includes a gypenoside. Some specific gypenosides include, but are not limited to CP-1-6.

According to a specific embodiment, the active ingredient or combination thereof includes a volatile compound, e.g., malonic acid, benzyl-O-beta-D-glucopyranoside, lutein, vomifoliol, palmitic acid, linoleic acid.

According to a specific embodiment, the active ingredient or combination thereof includes a phytosterol, e.g., stigmasterol, ergostane.

According to a specific embodiment, the active ingredient or combination thereof includes a flavenoid, e.g., Kaempferol, quercetin, rutin.

According to a specific embodiment, the active ingredient or combination thereof includes a phenolic compound.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 100 or 75%; 5 hours in Soxhlet apparatus, or 50% acetone extraction and 75% ethanol extraction: 2 g sample in 20 ml solvent at ambient temperature and filtration through 45 micron filter. Other extraction procedures include, but are not limited to, those described in Yantao et al. 2016 Chi Med 11:43, which is hereby incorporated by reference in its entirety.

According to another embodiment, the plant part is leaf.

Also contemplated herein are plants of the genus Gynostemma.

### Origanum Syriacum

According to a specific embodiment, the plants of this species include flavones, monoterpenoids and monoterpenes. Over 60 different compounds have been identified, with the primary ones being carvacrol and thymol ranging to over 80%, while lesser abundant compounds include p-cymene, γ-terpinene, caryophyllene, spathulenol, germacrene-D, β-fenchyl alcohol and δ-terpineol.

The table below shows a profile of the organic compounds identified in Origanum extract through fractional distillation:

Profile of the organic compounds found in the fractions analyzed.

| | Boiling | | % de Relative Area | | | Unoil | | |
|---|---|---|---|---|---|---|---|---|
| Compound | Point ° C. | Code | F1 | F2 | F3 | F4 | | Ooil |
| α-thujene | 150-152 | MH1 | 5.03 | 0.389 | ND | ND | | 1.74 |
| α-pinene | 156 | MH2 | 3.01 | ND | ND | ND | ND | 1.07 |
| β-myrcene | 166-168 | MH3 | 11.62 | 6.93 | 1.08 | ND | ND | 5.50 |
| Phellandrene | 172 | MH4 | 1.32 | 1.00 | ND | ND | ND | 0.72 |
| α-terpinene | 174 | MH5 | 8.91 | 8.32 | 2.90 | ND | ND | 5.57 |
| o-cymene | 174 | MH6 | 47.96 | 53.97 | 38.14 | 1.31 | 0.973 | 39.13 |
| Limonene | 175 | MH7 | 2.29 | 2.71 | 1.25 | ND | ND | 1.58 |
| 1,8-cineole | 177 | MO1 | 1.51 | 1.77 | 2.74 | ND | ND | 1.53 |
| γ-terpinene | 181-183 | MH8 | 15.59 | 24.43 | 40.57 | 1.40 | 0.94 | 22.34 |
| Thymol | 232 | MO2 | ND | ND | ND | 5.08 | 3.77 | 1.71 |
| Carvacrol | 237-238 | MO3 | ND | ND | 4.58 | 60.03 | 64.31 | 12.60 |
| Trans- | 268 | SeH1 | ND | ND | 2.97 | 18.96 | 13.78 | 3.47 |
| caryophyllene | | | | | | | | |
| α-humulene | 276 | SeH2 | ND | ND | 0.34 | 6.16 | 8.36 | 1.56 |

| | | | | | | | |
|---|---|---|---|---|---|---|---|
| Monoterpene hydrocarbons | (MH) | 95.73 | 97.75 | 83.94 | 2.71 | 1.91 | 77.65 |
| Monoterpene oxygenated | (MO) | 1.51 | 1.77 | 7.32 | 65.11 | 68.08 | 15.84 |
| Sesquiterpene hydrocarbons | (SeH) | ND | ND | 3.31 | 25.12 | 22.14 | 5.03 |
| Total identified components | | 97.24 | 99.52 | 94.57 | 92.94 | 92.13 | 98.52 |

Oregano essential oil (Ooil) was obtained through the steam entrainment method and the oil fractions through a fractional distillation system. The first fraction started to distill at a temperature of 82° C. and the last fraction distilling at 140° C., finally undistilled oil (Unoil) was obtained. At the end of the process, five fractions named Fraction 1 (F1), Fraction 2 (F2), Fraction 3 (F3), Fraction 4 (F4), and undistilled oil (Unoil) were obtained.

When Origanum extract was analyzed on HPLC, a variety of phenolic compounds were identified:

Phenolic compounds determined by the HPLC method in O. vulgare ssp. vulgare extract.

| | | Retention | UV | MS | Con- |
|---|---|---|---|---|---|
| | [M-H] | Time | Detec- | Detec- | centration |
| Compounds | m/z | (t_{R}), min | tion | tion | (mg/g) |
| Gentisic acid | 153 | 3.69 ± 0.04 | NO | YES | <0.02 |
| Chlorogenic | 353 | 6.43 ± 0.05 | YES | YES | 2.10 ± 0.14 |
| acid | | | | | |
| p-Coumaric | 163 | 9.48 ± 0.08 | NO | YES | <0.02 |
| acid | | | | | |
| Hyperoside | 463 | 18.60 ± 0.12 | YES | YES | 1.05 ± 0.03 |
| Isoquercitrin | 463 | 20.29 ± 0.10 | YES | YES | 0.71 ± 0.19 |
| Rutin | 609 | 20.76 ± 0.15 | YES | YES | 0.64 ± 0.15 |
| Rosmarinic | 360 | 21.80 ± 0.10 | YES | YES | 12.83 ± 2.19 |
| acid | | | | | |
| Quercitrin | 447 | 23.64 ± 0.13 | YES | YES | 0.50 ± 0.08 |
| Quercetin | 301 | 27.55 ± 0.15 | NO | YES | <0.02 |
| Luteolin | 285 | 29.64 ± 0.19 | YES | YES | 0.10 ± 0.04 |

Values are the mean ± SD (n = 3).

Total polyphenol content and antioxidant activity of O. vulgare ssp. vulgare extract.

| | TPC | Flavonoid | Caffeic Acids | CUPRAC | FRAP | SO Scavengin |
|---|---|---|---|---|---|---|
| Sample | (mg GAE/g) | (mg RE/g) | (mg CAE/g) | (μM TE/g) | (μM TE/g) | (μM TE/g) |
| O. vulgare | 94.69 ± 4.03 | 38.46 ± 3.54 | 29.92 ± 1.08 | 1284 ± 66 | 794.40 ± 25.80 | 44.00 ± 0.56 |

Each value is the mean ± SD of three independent measurements. TPC, total polyphenols content; SO, superoxide; GAE, gallic acid equivalents; RE, rutin equivalents; CAE, caffeic acid equivalents; TE, Trolox equivalents.

Also contemplated herein are plants of the genus Origanum.

Origanum is a genus of herbaceous perennials and subshrubs in the family Lamiaceae, native to Europe, North Africa, and much of temperate Asia, where they are found in open or mountainous habitats. A few species also naturalized in scattered locations in North America and other regions.

The plants have strongly aromatic leaves and abundant tubular flowers with long-lasting coloured bracts. The genus includes the important group of culinary herbs: marjoram (Origanum majorana) and oregano (Origanum vulgare).

Examples include, but are not limited to:
Origanum acutidens (Hand.-Mazz.) letsw.—Turkey, Iraq
Origanum×adanense Baser & H. Duman-Turkey (O. bargyli×O. laevigatum)
Origanum×adonidis Mouterde—Lebanon (O. libanoticum×O. syriacum subsp. bevanii)
Origanum akhdarense letsw. & Boulos—Cyrenaica region of eastern Libya
Origanum amanum Post—Hatay region of Turkey
Origanum×barbarae Bornm.—Lebanon (O. ehrenbergii×O. syriacum subsp. bevanii)
Origanum bargyli Mouterde—Turkey, Syria
Origanum bilgeri P. H. Davis—Antalya region of Turkey
Origanum boissieri letsw.—Turkey
Origanum calcaratum Juss.—Greece
Origanum compactum Benth.—Spain, Morocco
Origanum cordifolium (Montbret & Aucher ex Benth.) Vogel—Cyprus
Origanum cyrenaicum Bég. & Vacc.—Cyrenaica region of eastern Libya
Origanum dayi Post—Israel
Origanum dictamnus L.—hop marjoram, Cretan dittany, dittany of Crete-endemic to Crete
Origanum×dolichosiphon P. H. Davis—Seyhan region of Turkey (O. amanum×O. laevigatum)
Origanum ehrenbergii Boiss.—Lebanon
Origanum elongatum (Bonnet) Emb. & Maire—Morocco
Origanum floribundum Munby—Algeria
Origanum×haradjanii Rech.f—Turkey (O. laevigatum×O. syriacum subsp. bevanii)
Origanum haussknechtii Boiss.—Turkey
Origanum husnucan-baseri H. Duman, Aytac & A. Duran—Turkey
Origanum hypericifolium O. Schwarz & P. H. Davis—Turkey
Origanum×intercedens Rech.f.—Greece, Turkey (O. onites×O. vulgare subsp. hirtum)
Origanum×intermedium P. H. Davis—Denizli region of Turkey (O. onites×O. sipyleum)
Origanum isthmicum Danin—Sinai
Origanum jordanicum Danin & Kunne—Jordan
Origanum laevigatum Boiss.—Turkey, Syria, Cyprus
Origanum leptocladum Boiss.—Turkey
Origanum libanoticum Boiss.—Lebanon
Origanum majorana L.—(sweet) marjoram—Turkey, Cyprus; naturalized in scattered locations in Europe, North Africa, North+South America
Origanum×lirium Heldr. ex Halácsy—Greece (O. scabrum×O. vulgare subsp. hirtum)
Origanum×majoricum Cambess.—hardy sweet marjoram—Spain including Balearic Islands (O. majorana×O. vulgare subsp. virens)
Origanum microphyllum (Benth.) Vogel—Crete
Origanum×minoanum P. H. Davis—Crete (O. microphyllum×O. vulgare subsp. hirtum)
Origanum minutiflorum O. Schwarz & P. H. Davis—Turkey
Origanum munzurense Kit Tan & Sorger—Turkey
Origanum×nebrodense Tineo ex Lojac-Sicily (O. majorana×O. vulgare subsp. viridulum)
Origanum onites L.—Greece, Turkey, Sicily
Origanum×pabotii Mouterde—Syria (O. bargyli×O. syriacum subsp. bevanii)
Origanum pampaninii (Brullo & Furnari) letsw—Cyrenaica region of eastern Libya
Origanum petraeum Danin—Jordan
Origanum punonense Danin—Jordan
Origanum ramonense Danin—Israel
Origanum rotundifolium Boiss.—Turkey, Caucasus
Origanum saccatum P. H. Davis—Turkey
Origanum scabrum Boiss. & Heldr. in P. E. Boissier—Greece
Origanum sipyleum L.—Turkey, Greek Islands
Origanum solymicum P. H. Davis—Antalya region of Turkey
Origanum symes Carlström-Islands of the Aegean Sea
Origanum syriacum L.—Turkey, Cyprus, Syria, Lebanon, Jordan, Palestine, Israel, Sinai, Saudi Arabia
Origanum vetteri Briq. & Barbey—Crete
Origanum vogelii Greuter & Burdet—Turkey
Origanum vulgare L.—oregano—Europe, North Africa, temperate Asia (Iran, Siberia, Central Asia, China, etc.); naturalized in parts of North America, New Zealand, Venezuela.

According to a specific embodiment, the active ingredient or combination thereof includes an organic compound component of Origanum extract.

According to a specific embodiment, the active ingredient or combination thereof is selected from the group consisting of α-thujene α-pinene, β-myrcene, Phellandrene, α-terpinene, o-cymene, Limonene, 1,8-cineole, γ-terpinene, Thymol, Carvacrol, Trans-caryophyllene and a-humulene.

According to a specific embodiment, the active ingredient or combination thereof includes a monoterpene hydrocarbon, an oxygenated monoterpene and a sesquiterpene hydrocarbon.

According to a specific embodiment, the active ingredient or combination thereof includes a phenolic compound, e.g., gentisic acid, chlorogenic acid, p-coumaric acid, hyperoside, isoquercitrin, rutin, rosmarinic acid, quercirtin, quercetin and luteolin.

According to a specific embodiment, the active ingredient or combination thereof includes a mineral, e.g., potassium, calcium, magnesium, phosphorus, aluminum, iron, sodium, boron, zinc, cadmium, selenium.

### Sesame

Sesame seeds contain thelignans, sesamolin, sesamin, pinoresinol andlariciresinol. Insoluble 11S globulin and soluble 2S albumin, conventionally termed α-globulin and β-globulin, are the two major storage proteins and constitute 80-90% of total seed proteins in sesame. Comparison of amino acid composition indicated that they are substantially less hydrophobic than the known oleosins, and thus should not be aggregated multimers of oleosins. The results of immuno-recognition to sesame proteins reveals that these three polypeptides are unique proteins gathered in oil bodies, accompanying oleosins and triacylglycerols, during the active assembly of the organelles in maturing seeds. The phospholipid, oleic and linoleic acids, chlorophyll and sesamolin, sesamol and γ-tocopherol are found. 10 compounds [2-furfurylthiol, 2-phenylethylthiol, 2-methoxyphenol, 4-hydroxy2,5-dimethyl-3 [2H]-furanone, 2-pentylpyridine, 2-ethyl-3,5-dimethylpyrazine, acetylpyrazine, [E,E]-2,4-decadienal, 2-acetyl-1-pyrroline and 4-vinyl-2-methoxy-phenol] are quantified. On the basis of high OAVs in oil, especially 2-acetyl-1-pyrroline [roasty], 2-furfurylthiol [coffee-like], 2-phenylethylthiol [rubbery] and 4-hydroxy-2,5-dimethyl3 [2H]-furanone [caramel-like] are elucidated as important contributors to the overall roasty, sulphury odour of the crushed sesame material. The structures of novel sesaminol glucosides isolated from sesame seed are determined to be sesaminol 2′-O-β-d-glucopyranoside, sesaminol 2′-O-β-d-glucopyranosyl [1→2]-O-β-dglucopyranoside and sesaminol 2′-O-β-d-glucopyranosyl [1>>2]-O-[β-d-glucopyransyl [1>>6]]-[β-dglucopyranoside. Also minor sesame lignans such as -(7S,8′R,8R)-acuminatolide piperitol and pinoresinol (as mentioned).

Also contemplated herein are plants of the genus Sesamum.

Examples include, but are not limited to:
Sesamum abbreviatum Merxm.
Sesamum alatum Thonn.
Sesamum angolense Welw.
Sesamum biapiculatum De Wild.
Sesamum calycinum Welw.
Sesamum capense Burm. f.
Sesamum digitaloides Welw. ex Schinz
Sesamum gracile Endl.
Sesamum hopkinsii Suess.
Sesamum indicum L.
Sesamum lamiifolium Engl.
Sesamum latifolium J. B. Gillett
Sesamum lepidotum Schinz
Sesamum macranthum Oliv.
Sesamum marlothii Engl.
Sesamum mombazense De Wild. & T. Durand
Sesamum parviflorum Seidenst.
Sesamum pedalioides Welw. ex Hiern
Sesamum radiatum Schumach. & Thonn.
Sesamum rigidum Peyr.
Sesamum rostratum Hochst.
Sesamum sabulosum A. Chev.
Sesamum schinzianum Asch.
Sesamum somalense Chiov.
Sesamum thonneri De Wild. & T. Durand
Sesamum triphyllum Welw. ex Asch.

Plants that contain Lignan according to some embodiments of the invention include a wide variety of plant foods, including seeds (flax, pumpkin, sunflower, poppy, sesame), whole grains (rye, oats, barley), bran (wheat, oat, rye), beans, fruit (particularly berries), and vegetables (Broccoli and curly kale are rich sources of lignans. Other vegetables such as white and red cabbage, Brussels sprouts, cauliflower, carrots, green and red sweet peppers are also good sources).

Additional plants that contain Sesamin include but are limited to Eleutherococcus senticosus.

Thus, any combination of the above plants is contemplated including 2, 3, 4, 5, 6, 7 of the plants. According to another embodiment, a combination of extracts or fractions including 2, 3, 4, 5, 6, 7 of the different plants.

Examples include, but are not limited to, Nigella sativa, Thymus vulgaris, Origanum syriacum, Thymbra spicata, Satujera thymbra, Sesamum indicum and Rhus coriaria.

Nigella sativa, Thymus capitatus, Origanum syriacum, Thymbra spicata, Satujera thymbra, Sesamum indicum and Rhus coriaria.

Nigella sativa, Thymus capitatus, Thymus vulgaris, Thymbra spicata, Satujera thymbra, Sesamum indicum and Rhus coriaria.

Nigella sativa, Thymus capitatus, Thymus vulgaris, Origanum syriacum, Satujera thymbra, Sesamum indicum and Rhus coriaria.

Nigella sativa, Thymus capitatus, Thymus vulgaris, Origanum syriacum, Thymbra spicata, Sesamum indicum and Rhus coriaria.

Nigella sativa, Thymus capitatus, Thymus vulgaris, Origanum syriacum, Thymbra spicata, Satujera thymbra, and Rhus coriaria.

Nigella sativa, Thymus capitatus, Thymus vulgaris, Origanum syriacum, Thymbra spicata, Satujera thymbra, Sesamum indicum.

Nigella sativa, Origanum syriacum, Thymbra spicata, Satujera thymbra, Sesamum indicum and Rhus coriaria.

Nigella sativa, Thymus capitatus, Thymus vulgaris, Satujera thymbra, Sesamum indicum and Rhus coriaria.

Nigella sativa, Thymus capitatus, Thymus vulgaris, Origanum syriacum, Sesamum indicum and Rhus coriaria.

Nigella sativa, Thymus capitatus, Thymus vulgaris, Origanum syriacum, Thymbra spicata, and Rhus coriaria.

Nigella sativa, Thymus capitatus, Thymus vulgaris, Origanum syriacum, Thymbra spicata, Satujera thymbra.

Nigella sativa, Thymus capitatus.

Nigella sativa, Thymus vulgaris.

Nigella sativa, Origanum syriacum.

Nigella sativa, Thymbra spicata.

Nigella sativa, Satujera thymbra.

Nigella sativa, Sesamum indicum.

Nigella sativa, Rhus coriaria.

Also contemplated are various combinations without Nigella sativa.

According to another embodiment, a combination of active ingredients e.g., thymoquinone, carvacrol, thymol; thymoquinone, carvacrol; thymoquinone, thymol; carvacrol, thymol.

Nigella sativa, Thymus capitatus, Thymus vulgaris.

Nigella sativa, Thymus vulgaris, Origanum syriacum.

Nigella sativa, Origanum syriacum, Thymbra spicata.

Nigella sativa, Thymbra spicata, Satujera thymbra.

Nigella sativa, Satujera thymbra, Sesamum indicum Rhus coriaria.

According to some embodiments the plants and active ingredients thereof are listed in the Table below.

| Origanum | Carvacrol |
|---|---|
| Syricaum | Thymol |
| Thymus Capitatus | Carvacrol |
| | p-cymene |
| | y-terpinene |
| | b-caryophyllene |
| Thymus Vulgaris | Thymol |
| Thymbra Spicata | Carvacrol |
| | y-terpinene |
| | p-cymene |
| Satureja Thymbra | y-terpinene |
| | p-cymene |
| | carvacrol |
| | thymol |
| Sumac | Tannin |
| Seasame | Lignans |
| | Seasamolin |
| | Seasamin |
| | Pinoresinol |
| | Lariciresinol |
| Nigella sativa | Thymoquinone |

Other embodiments, which comprise any of the Nigella sativa, Thymus capitatus, Thymus vulgaris, Origanum syriacum, Thymbra spicata, Satujera thymbra, Sesamum indicum, Rhus coriaria, Panax ginseng and Gynostemme pentaphyllum plants or grenera thereof in combinations of 2, 3, 4, 5, 6, 7 and 8 plants are contemplated herein.

### Mango

Mango fruit components can be grouped into macronutrients (carbohydrates, proteins, amino acids, lipids, fatty, and organic acids), micronutrients (vitamins and minerals), and phytochemicals (phenolic, polyphenol, pigments, and volatile constituents). Mango fruit has the following make up (M. Maldonado-Celis et al. Front Plant Sci. (2019) 10:1073).

### Amino Acid Compostion

| Amino acid | Content of g/100 g |
|---|---|
| Isoleucine | 0-0.029 |
| Leucine | 0-0.050 |
| Lysine | 0-0.066 |
| Methionine | 0-0.008 |
| Phenylalanine | 0-0.027 |
| Tyrosine | 0-0.016 |
| Tryptophan | 0-0.013 |
| Threonine | 0-0.031 |
| Valine | 0-0.042 |
| Histidine | 0-0.019 |
| Arginine | 0-0.031 |
| Alanine | 0-0.082 |
| Aspartic acid | 0-0.068 |
| Glutamic acid | 0-0.096 |
| Glycine | 0-0.034 |
| Proline | 0-0.029 |
| Serine | 0-0.035 |

### Content of Fatty Acids in Mango Fruit

| Carbon | Common | | | Part of |
|---|---|---|---|---|
| skeleton | name | Variety | Content | the fruit |
| 16:0^{a} | Palmitic acid | Malaysia | 6.95-10.93 | Seed |
| | | Mixed Egypt | 5.8 | |
| | | Manila Mexico | 9.29 | |
| | | Kaew Thailand | 5.4 | |
| | | 4 varieties Kenya | 4.87-10.57 | |
| 18:0^{a} | Stearic acid | Malaysia | 32.8-47.62 | Seed |
| | | Mixed Egypt | 38.3 | |
| | | Manila Mexico | 39.07 | |
| | | Kaew Thailand | 46.6 | |
| | | 4 varieties Kenya | 24.22-32.80 | |
| 20:0^{a} | Arachidic acid | Malaysia | 1 77-2.43 | Seed |
| | | Mixed Egypt | — | |
| | | Manila Mexico | 2.48 | |
| | | Kaew Thailand | 1.7 | |
| | | 4 varieties Kenya | 0.67-1.64 | |
| 24:0^{a} | Lignoceric acid | — | — | Seed |
| 18:1 (Δ⁹)^{a} | Oleic acid | Malaysia | 37.01-47.28 | Seed |
| | | Mixed Egypt | 46.1 | |
| | | Manila Mexico | 40.81 | |
| | | Kaew Thailand | 41.1 | |
| | | 4 varieties Kenya | 46.37-58.59 | |
| 18:2 (Δ^{9,12})^{a} | Linoleic acid | Malaysia | 3.66-6.87 | Seed |
| | | Mixed Egypt | 8.2 | |
| | | Manila Mexico | 6.06 | |
| | | Kaew Thailand | 3.8 | |
| | | 4 varieties Kenya | 6.73-10.4 | |
| 18:3 (Δ^{9,12,15})^{a} | α-Linoleic acid | — | — | Seed |
| 14:0^{b} | Myristic acid | Alphonso | 174.29, 231.21 | Pulp, peel |
| | | Pairi | 74.03, 295.16 | |
| | | Kent | 40.57, 323.9 | |
| 16:0^{b} | Palmitic acid | Alphonso | 1,933.43, 2,682.16 | Pulp, peel |
| | | Pairi | 896, 3,460.13 | |
| | | Kent | 560.88, 2,883.29 | |
| 18:0^{b} | Stearic acid | Alphonso | 75.63, 123.57 | Pulp, peel |
| | | Pairi | 33.36, 238.57 | |
| | | Kent | 29.76, 116.39 | |
| 20:0^{b} | Arachidic acid | Alphonso | 19.01, 29.21 | Pulp, peel |
| | | Pairi | 7.2, 55.24 | |
| | | Kent | 3.2, 32.56 | |
| 22:0^{b} | Behenic acid | Alphonso | 24.90, 43.83 | Pulp, peel |
| | | Pairi | 8.88, 55.38 | |
| | | Kent | 3.67, 43.73 | |
| 24:0^{b} | Lignoceric acid | Alphonso | 35.85, 86.16 | Pulp, peel |
| | | Pairi | 27.04, 1,17.24 | |
| | | Kent | 24.88, 71.15 | |
| 16:1, n-7^{b} | Palmitoleic acid | Alphonso | 2,881.90, 1,986.59 | Pulp, peel |
| | | Pairi | 599.84, 533.59 | |
| | | Kent | 314.28, 1,527.72 | |
| 16:1, n-5^{b} | 11-Hexadecenoic acid | Alphonso | 146.22, 119.07 | Pulp, peel |
| | | Pairi | 51.49, 58.01 | |
| | | Kent | 22.42, 147.05 | |
| 17:1, n-7^{b} | 10-Heptadecenoic acid | Alphonso | 11.82, n.d. | Pulp, peel |
| | | Pairi | 8.76, n.d. | |
| | | Kent | 3.76, n.d. | |
| 18:1, n-9^{b} | Oleic acid | Alphonso | 856.59, 2,376.3 | Pulp, peel |
| | | Pairi | 761.79, 2,847.25 | |
| | | Kent | 261.3, 778.48 | |
| 18:1, n-7^{b} | 11-Octadecenoic acid | Alphonso | 646.48, 480.59 | Pulp, peel |
| | | Pairi | 248.78, 321.16 | |
| | | Kent | 176.61, 282.14 | |
| 20:1, n-9^{b} | 11-Eicosenoic acid | Alphonso | 6.57, 10.01 | Pulp, peel |
| | | Pairi | 2.39, 10.49 | |
| | | Kent | n.d., n.d. | |
| 16:2, n-4 | 9,12-Hexadecadienoic acid | Alphonso | 33.86, n.d. | Pulp, peel |
| | | Pairi | 17.71, n.d. | |
| | | Kent | 16.09, n.d. | |
| 18:2, n-6 | Linoleic acid | Alphonso | 83.58, 422.83 | Pulp, peel |
| | | Pairi | 139.44, 1,956.03 | |
| | | Kent | 80,05, 1,277.41 | |
| 18:2, n-3 | 9,15-Octadecadienoic acid | Alphonso | 61.58, n.d | Pulp, peel |
| | | Pairi | 20.24, n.d. | |
| | | Kent | 20.93, n.d. | |
| 7:2, n-3 | Hepta-2,4(E,E)-dienoic acid | Alphonso | 698.01, 265.93 | Pulp, peel |
| | | Pairi | 662.32, 1,152.72 | |
| | | Kent | 835.33, 352.98 | |
| 18:3, n-3 | Linolenic acid | Alphonso | 840.37, 1,149.88 | Pulp, peel |
| | | Pairi | 522.23, 1,991.68 | |
| | | Kent | 408.42, 1,201.18 | |

Vitamin composition in 100 g of edible portion of mango fruit (United States Department of Agriculture, Agricultural Research Service, 2018)

| Vitamin | Value per 100 g | |
|---|---|---|
| Ascorbic acid (Vit C) | 13.2-92.8 | mg |
| Thiamine (Vit B1) | 0.01-0.04 | mg |
| Riboflavin (Vit B2) | 0.02-0.07 | mg |
| Niacin (Vit B3) | 0.2-1.31 | mg |
| Pantothenic acid (Vit B5) | 0.16-0.24 | mg |
| Pyridoxine (Vit B6) | 0.05-0.16 | mg |
| Folate total | 20-69 | μg |
| Folic acid | 0 | μg |
| Folate food | 20-69 | μg |
| B12 | 0.00 | mg |
| Vitamin A | 54 | μg |
| Vitamin B (α-tocopherol) | 0.79-1.02 | mg |
| Vitamin K | 4.2 | μg |

### Mineral Composition in Edible Portion of Mango Fruit

| Mineral | Value (mg) per 100 g^{a} | Value (mg) per 100 g^{b} |
|---|---|---|
| Calcium | 7-16 | 9-21 |
| Iron | 0.09-0.41 | 0.1-0.9 |
| Magnesium | 8-19 | 10-38 |
| Phosphorus | 10-18 | 19-23 |
| Potassium | 120-211 | 147-617 |
| Sodium | 0-3 | 0-4 |
| Zinc | 0.06-0.15 | 0-0.1 |
| Copper | 0.04-0.32 | n.d. |
| Manganese | 0.03-0.12 | 1.6-18.2 |
| Selenium | 0-0.6 | n.d. |

^{a}United States Department of Agriculture, Agricultural Research Service, (2018).
^{b}Instituto Colombiano de Bienestar Familiar (ICBF), (2015).

### Mango Fruit Also Contains:

### Phenolic Acids

Mango pulp includes the two major categories of phenolic acids in plants, hydroxybenzoic and hydroxycinnamic acid derivatives. The phenolic acids may be present free or conjugated forms with glucose or quinic acid (Mattila and Kumpulainen, 2002, Burton-Freeman et al., 2017). Hydroxybenzoic acids detected in the mango pulp are gallic, vanillic, syringic, protocatechuic, and p-hydroxybenzoic acids, while the hydroxycinnamic acid derivatives are p-coumaric, chlorogenic, ferulic, and caffeic acids (Masibo and Qian, 2008; Ediriweera et al., 2017).

The following phenolic acids were identified in the flesh and skin of nine mango varieties cultivated in China (Abbasi et al., 2015). In 100 g FW of pulp: ferulic acid (33.75 mg), protocatechuic (0.77 mg), chlorogenic (0.96-6.20 mg), gallic (0.93-2.98 mg), vanillic (0.57-1.63 mg), and caffeic acids (0.25-0.10 mg) (Abbasi et al., 2015). The major phenolic acids in Ataulfo mango from Mexico were protocatechuic acid (0.48-1.1 mg/100 g dry weight (DW)), vanillic acid (16.9-24.4 mg/100 g DW), gallic acid (94.6-98.7 mg/100 g DW), and chlorogenic acid (28-301 mg/100 g DW) (Palafox-Carlos et al., 2012a; Palafox-Carlos et al., 2012b). The peel extracts of the mango cultivars Ataulfo, Keitt, Osteen, and Sensation have been found to have high concentrations of phenolic acids and derivatives such as gallic, syringic, methyl digallate ester, methyl gallate, gallotannins, galloyl glucose, theogallin, protocatechuic, and ferulic acid (Gómez-Caravaca et al., 2015; López-Cobo et al., 2017; Pacheco-Ordaz et al., 2018).

### Flavonoids and Other Polyphenolic Compounds

The most abundantly occurring polyphenols in plants are flavonoids, stilbenes, and lignans, of which flavonoids account for 60% of dietary polyphenols (Ramos, 2007; Van Breda et al., 2008). Polyphenols in the mango fruit include catechins, quercetin, kaempferol, rhamnetin, anthocyanins, and tannic acid and the class of xanthones: mangiferin (Manach et al., 2004; Masibo and Qian, 2008). The pulp of mango contains glycosides of quercetin (glucose, galactose, rhamnose, xylose, and arabinose), with kaempferol, isorhamnetin, fisetin, and myricetin being present in minor levels (Berardini et al., 2003; Ribeiro et al., 2008; Ramirez et al., 2013; USDA, 2018).

The USDA Nutrient Data Laboratory Flavonoid Database (https://www.ars.usda.gov/northeast-area/beltsville-md-bhnrc/beltsville-human-nutrition-research-center/nutrient-data-laboratory/) list the following in 100 g of edible portion of mango fruit: anthocyanidins (cyanidin, 0.10 mg; delphinidin 0.02 mg; and pelargonidin, 0.02 mg), the flavan-3-ol (+)-catechin (1.72 mg), traces of the flavones apigenin (0.01 mg) and luteolin (0.02), the flavonols kaempferol (0.05 mg) and myricetin (0.06 mg) (Haytowitz et al., 2018). In addition, the Nutrient Database lists the following in mango fruit (Tommy Atkins, Kent, Keitt, and Haden) isoflavones (0.01 mg), proanthocyanidins dimers (1.8 mg), trimers (1.4), and four to six dimers (7.2 mg). Thus, the main flavonoids that have detected in mango flesh are quercetin and glycosides derivatives; with the most relevant being flavonol glycoside quercetin 3-galactoside (22.1 mg/kg), followed by quercetin 3-glucoside (16.0 mg/kg), quercetin 3-arabinoside (5.0 mg/kg), and quercetin aglycone (3.5 mg/kg) (Ediriweera et al., 2017; Matheyambath et al., 2016). Some mango cultivars grown in Thailand (Tommy Atkins, Mani, Ngowe, R2E2, Kent, Jose, Mini-mango, Haden, Heidi, and Kaew Mon Duen Gao) contain glycosides of quercetin between 3.5 and 1,309.1 mg/100 g fruit (diglycoside, 3-O-gal, 3-O-glc, 3-O-xyl, 3-O-arap, 3-O-araf, and 3-O-rha), kaempferol 3-glc (6.7-77.3 mg), rhamnetin-3-O-gal/glc (5.4-734.4 mg), and quercetin (1.7-19.3 mg) (Berardini et al., 2003).

The seed and peel of mango fruit are also contain polyphenols (Ribeiro et al., 2007; Ribeiro et al., 2008), with a total phenolic content for these residues of 6-8% of DM in Uba cultivar from Brazil, which is 4.6 and 7.3 times higher, respectively, than the content of the pulp, and a similar profile was reported for the flavonoids and xanthones of this variety (Ribeiro et al., 2008).

Xanthones are molecules formed by a C6-C3-C6 backbone structure with hydroxyl, methoxyl, and isoprene units linked to the A and B rings, generally occurring as ethers or glycosides (Negi et al., 2013). Six xanthone derivatives have been identified (mangiferin, dimethyl mangiferin, homomangiferin, mangiferin gallate, isomangiferin, and isomangiferin gallate); among this group mangiferin (C2-b-d-glucopyranosyl-1,3,6,7-tetrahydroxyxanthone), a C-glucosyl xanthone, is broadly distributed in higher plants, with demonstrated pharmacological and antioxidant activities. Mangiferin can be obtained from the bark, fruits, roots, and leaves of Mangifera indica Linn (Matheyambath et al., 2016).

The content of mangiferin and derivatives is higher in the peel from Pica and Tommy Atkins mango fruit (22.15 and 9.68 mg/100 g FW, respectively) than in the pulp, 4.24 and 3.25 mg/100 g FW, respectively (Ramirez et al., 2013). In Uba and Tommy Atkins cultivars from Brazil, mangiferin was detected as 12.4 and 2.9 mg/kg DM, respectively, but it was not detected in Palmer pulp (Ribeiro et al., 2008).

Mangos have also been shown to have high levels of choline, folate and beta carotene.

Of particular interest from mango are vitamins B and B-12, as well as magnesium and choline.

Any of the compositions (i.e., plant part, extract thereof, fraction thereof, active ingredient thereof, synthetic analog thereof, mimetic thereof or combination thereof) described herein can be used for the treatment of inflammation. In some embodiments of the present invention there is provided compositions or food supplements comprising Bromelain or pineapple extracts comprising Bromelain.

In some embodiments of the present invention there is provided compositions or food supplements comprising tryptophan, analogs of tryptophan or extract of paint containing tryptophan such as sesame or oregano.

In some embodiments of the present invention there is provided method, vaccine, pharmaceutical composition, composition or food supplement further including “Beduin Tea” comprising at least 3 of dried thyme, dried sage, cardomon pods, cinnamon stick, black tea, Habuk and Marmaraya.

In some embodiments the present invention is directed to a composition having at least the components of one of Complex A to Complex D.

| Component No. | Component |
|---|---|
| 1 | Oregano Oil |
| 2 | Thyme Oil |
| 3 | Nigela Sativa |
| 4 | Sumac Oil |
| 5 | Sesame Oil |
| 6 | Olibanum Oil |
| Complex A | Oils 1 + 2 + 3 |
| Complex B | Oils 1 + 2 + 3 + 4 |
| Complex C | Dils 1 + 2 + 3 + 4 + 5 |
| Complex D | Oils 1 + 2 + 3 + 4 + 5 + 6 |

1. It should be understood that the compositions are not limited to the components No. 1-6 and the compositions may contain additional active agents and/or e.g. flavor enhancers, delivery agents, stabilizers etc. For example, the activity of the Complexes A-D may be enhanced by the addition of further natural products having specific activity. An example, of this would be to include components from one of more of mango plants/fruits; ginsing, pineapple fruit extracts, tryptophan, silibinin (which may be included from an extract of milk thistle), chamomile, anise or star anise, Bedouin tea, α-bisabolol (which may be included from an extract of chamomile), linalool (which may be included from an extract of e.g. lavender, bergamot, rosewood, basil or neroli oil), turmeric or extract thereof, mango ginger or an extract thereof and curcumin, etc.

The term “treating” refers to inhibiting, preventing or arresting the development of regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for-, or predisposed to the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein “inflammation” refers to a part of the complex biological response of body tissues to harmful stimuli, such as pathogens, damaged cells, or irritants,^{[1]} and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair.

The five classical signs of inflammation are heat, pain, redness, swelling, and loss of function. Inflammation is a generic response, and therefore it is considered as a mechanism of innate immunity, as compared to adaptive immunity, which is specific for each pathogen. Too little inflammation could lead to progressive tissue destruction by the harmful stimulus (e.g. bacteria) and compromise the survival of the organism. In contrast, chronic inflammation is associated with various diseases, such as hay fever, periodontal disease, atherosclerosis, and osteoarthritis.

Inflammation can be classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

As used herein “inflammatory disease” refers to a medical condition in which inflammation takes a role in onset or progression.

According to a specific embodiment, the inflammatory disease comprises an autoimmune disease.

According to a specific embodiment, the inflammatory disease comprises an acute inflammatory disease.

According to a specific embodiment, the inflammatory disease comprises a chronic inflammatory disease.

Inflammatory diseases-Include, but are not limited to, chronic inflammatory diseases and acute inflammatory diseases.

Inflammatory diseases associated with hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, psoriasis, rheumatoid autoimmune diseases, rheumatoid arthritis (Krenn V. et al., Histol Histopathol 2000 July; 15 (3): 791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2): 49), sclerosis, systemic sclerosis (Renaudineau Y. et al., Clin Diagn Lab Immunol. 1999 March; 6 (2): 156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl: S125), thyroid diseases, autoimmune thyroid diseases, Graves' disease (Orgiazzi J. Endocrinol Metab Clin North Am 2000 June; 29 (2): 339), thyroiditis, spontaneous autoimmune thyroiditis (Braley-Mullen H. and Yu S, J Immunol 2000 Dec. 15; 165 (12): 7262), Hashimoto's thyroiditis (Toyoda N. et al., Nippon Rinsho 1999 August; 57 (8): 1810), myxedema, idiopathic myxedema (Mitsuma T. Nippon Rinsho. 1999 August; 57 (8): 1759); autoimmune reproductive diseases, ovarian diseases, ovarian autoimmunity (Garza K M. et al., J Reprod Immunol 1998 February; 37 (2): 87), autoimmune anti-sperm infertility (Diekman A B. et al., Am J Reprod Immunol. 2000 March; 43 (3): 134), repeated fetal loss (Tincani A. et al., Lupus 1998; 7 Suppl 2: S107-9), neurodegenerative diseases, neurological diseases, neurological autoimmune diseases, multiple sclerosis (Cross A H. et al., J Neuroimmunol 2001 Jan. 1; 112 (1-2): 1), Alzheimer's disease (Oron L. et al., J Neural Transm Suppl. 1997; 49:77), myasthenia gravis (Infante A J. And Kraig E, Int Rev Immunol 1999; 18 (1-2): 83), motor neuropathies (Kornberg A J. J Clin Neurosci. 2000 May; 7 (3): 191), Guillain-Barre syndrome, neuropathies and autoimmune neuropathies (Kusunoki S. Am J Med Sci. 2000 April; 319 (4): 234), myasthenic diseases, Lambert-Eaton myasthenic syndrome (Takamori M. Am J Med Sci. 2000 April; 319 (4): 204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1): 23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, stoke, cardiovascular autoimmune diseases, atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16): 660); anti-factor VIII autoimmune disease (Lacroix-Desmazes S. et al., Semin Thromb Hemost.2000; 26 (2): 157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3): 178); antiphospholipid syndrome (Flamholz R. et al., J Clin Apheresis 1999; 14 (4): 171); heart failure, agonist-like β-adrenoceptor antibodies in heart failure (Wallukat G. et al., Am J Cardiol. 1999 Jun. 17; 83 (12A): 75H), thrombocytopenia purpura (Moccia F. Ann Ital Med Int. 1999 April-Jun; 14 (2): 114); hemolytic anemia, autoimmune hemolytic anemia (Efremov D G. et al., Leuk Lymphoma 1998 January; 28 (3-4): 285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1): 16), celiac disease (Landau Y E. and Shoenfeld Y. Harefuah 2000 Jan. 16; 138 (2): 122), autoimmune diseases of the musculature, myositis, autoimmune myositis, Sjogren's syndrome (Feist E. et al., Int Arch Allergy Immunol 2000 September; 123 (1): 92); smooth muscle autoimmune disease (Zauli D. et al., Biomed Pharmacother 1999 June; 53 (5-6): 234), hepatic diseases, hepatic autoimmune diseases, autoimmune hepatitis (Manns M P. J Hepatol 2000 Aug; 33 (2): 326) and primary biliary cirrhosis (Strassburg C P. et al., Eur J Gastroenterol Hepatol. 1999 June; 11 (6): 595).

Type IV or T cell mediated hypersensitivity, include, but are not limited to, rheumatoid diseases, rheumatoid arthritis (Tisch R, McDevitt HO. Proc Natl Acad Sci USA 1994 Jan. 18; 91 (2): 437), systemic diseases, systemic autoimmune diseases, systemic lupus erythematosus (Datta SK., Lupus 1998; 7 (9): 591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1): 77); ovarian diseases (Garza K M. et al., J Reprod Immunol 1998 February; 37 (2): 87), prostatitis, autoimmune prostatitis (Alexander R B. et al., Urology 1997 December; 50 (6): 893), polyglandular syndrome, autoimmune polyglandular syndrome, Type I autoimmune polyglandular syndrome (Hara T. et al., Blood. 1991 Mar. 1; 77 (5): 1127), neurological diseases, autoimmune neurological diseases, multiple sclerosis, neuritis, optic neuritis (Soderstrom M. et al., J Neurol Neurosurg Psychiatry 1994 May; 57 (5): 544), myasthenia gravis (Oshima M. et al., Eur J Immunol 1990 December; 20 (12): 2563), stiff-man syndrome (Hiemstra H S. et al., Proc Natl Acad Sci USA 2001 Mar. 27; 98 (7): 3988), cardiovascular diseases, cardiac autoimmunity in Chagas' disease (Cunha-Neto E. et al., J Clin Invest 1996 Oct. 15; 98 (8): 1709), autoimmune thrombocytopenia purpura (Semple J W. et al., Blood 1996 May 15; 87 (10): 4245), anti-helper T lymphocyte autoimmunity (Caporossi A P. et al., Viral Immunol 1998; 11 (1): 9), hemolytic anemia (Sallah S. et al., Ann Hematol 1997 March; 74 (3): 139), hepatic diseases, hepatic autoimmune diseases, hepatitis, chronic active hepatitis (Franco A. et al., Clin Immunol Immunopathol 1990 March; 54 (3): 382), biliary cirrhosis, primary biliary cirrhosis (Jones D E. Clin Sci (Colch) 1996 November; 91 (5): 551), nephric diseases, nephric autoimmune diseases, nephritis, interstitial nephritis (Kelly C J. J Am Soc Nephrol 1990 August; 1 (2): 140), connective tissue diseases, ear diseases, autoimmune connective tissue diseases, autoimmune ear disease (Yoo T J. et al., Cell Immunol 1994 August; 157 (1): 249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

According to a specific embodiment, the inflammatory disease comprises diabetes.

According to a specific embodiment, the diabetes comprises type I diabetes.

According to a specific embodiment, the diabetes comprises type II diabetes.

According to a specific embodiment, the diabetes comprises gestational diabetes.

### Autoimmune Diseases

Autoimmune diseases, include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), Wegener's granulomatosis, Takayasu's arteritis, Kawasaki syndrome (Praprotnik S. et al., Wien Klin Wochenschr 2000 Aug. 25; 112 (15-16): 660), anti-factor VIII autoimmune disease (Lacroix-Desmazes S. et al., Semin Thromb Hemost.2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3): 178), antiphospholipid syndrome (Flamholz R. et al., J Clin Apheresis 1999; 14 (4): 171), antibody-induced heart failure (Wallukat G. et al., Am J Cardiol. 1999 Jun. 17; 83 (12A): 75H), thrombocytopenia purpura (Moccia F. Ann Ital Med Int. 1999 April-Jun; 14 (2): 114; Semple J W. et al., Blood 1996 May 15; 87 (10): 4245), autoimmune hemolytic anemia (Efremov D G. et al., Leuk Lymphoma 1998 January; 28 (3-4): 285; Sallah S. et al., Ann Hematol 1997 March; 74 (3): 139), cardiac autoimmunity in Chagas' disease (Cunha-Neto E. et al., J Clin Invest 1996 Oct. 15; 98 (8): 1709) and anti-helper T lymphocyte autoimmunity (Caporossi A P. et al., Viral Immunol 1998; 11 (1): 9).

Examples of autoimmune rheumatoid diseases include, but are not limited to rheumatoid arthritis (Krenn V. et al., Histol Histopathol 2000 July; 15 (3): 791; Tisch R, McDevitt H O. Proc Natl Acad Sci units S A 1994 Jan. 18; 91 (2): 437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl: S125), autoimmune thyroid diseases, Graves' disease (Orgiazzi J. Endocrinol Metab Clin North Am 2000 June; 29 (2): 339; Sakata S. et al., Mol Cell Endocrinol 1993 March; 92 (1): 77), spontaneous autoimmune thyroiditis (Braley-Mullen H. and Yu S, J Immunol 2000 Dec. 15; 165 (12): 7262), Hashimoto's thyroiditis (Toyoda N. et al., Nippon Rinsho 1999 August; 57 (8): 1810), idiopathic myxedema (Mitsuma T. Nippon Rinsho. 1999 August; 57 (8): 1759), ovarian autoimmunity (Garza K M. et al., J Reprod Immunol 1998 February; 37 (2): 87), autoimmune anti-sperm infertility (Diekman A B. et al., Am J Reprod Immunol. 2000 March; 43 (3): 134), autoimmune prostatitis (Alexander R B. et al., Urology 1997 December; 50 (6): 893) and Type I autoimmune polyglandular syndrome (Hara T. et al., Blood. 1991 Mar. 1; 77 (5): 1127).

Examples of autoimmune gastrointestinal diseases include, but are not limited to, chronic inflammatory intestinal diseases (Garcia Herola A. et al., Gastroenterol Hepatol. 2000 January; 23 (1): 16). e.g. inflammatory bowel disease, bowel obstruction celiac disease (Landau Y E. and Shoenfeld Y. Harefuah 2000 Jan. 16; 138 (2): 122), colitis, ileitis, and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3): 382), primary biliary cirrhosis (Jones D E. Clin Sci (Colch) 1996 November; 91 (5): 551; Strassburg C P. et al., Eur J Gastroenterol Hepatol. 1999 June; 11 (6): 595) and autoimmune hepatitis (Manns M P. J Hepatol 2000 August; 33 (2): 326).

Examples of autoimmune neurological diseases include, but are not limited to, multiple sclerosis (Cross A H. et al., J Neuroimmunol 2001 Jan. 1; 112 (1-2): 1), Alzheimer's disease (Oron L. et al., J Neural Transm Suppl. 1997; 49:77), myasthenia gravis (Infante A J. And Kraig E, Int Rev Immunol 1999; 18 (1-2): 83; Oshima M. et al., Eur J Immunol 1990 December; 20 (12): 2563), neuropathies, motor neuropathies (Kornberg A J. J Clin Neurosci. 2000 May; 7 (3): 191); Guillain-Barre syndrome and autoimmune neuropathies (Kusunoki S. Am J Med Sci. 2000 April; 319 (4): 234), myasthenia, Lambert-Eaton myasthenic syndrome (Takamori M. Am J Med Sci. 2000 April; 319 (4): 204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7): 3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1): 23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5): 544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1): 92) and smooth muscle autoimmune disease (Zauli D. et al., Biomed Pharmacother 1999 June; 53 (5-6): 234) and fibromyalgia.

Examples of autoimmune nephric diseases include, but are not limited to, nephritis and autoimmune interstitial nephritis (Kelly C J. J Am Soc Nephrol 1990 August; 1 (2): 140).

Examples of autoimmune diseases related to reproduction include, but are not limited to, repeated fetal loss (Tincani A. et al., Lupus 1998; 7 Suppl 2: S107-9).

Examples of autoimmune connective tissue diseases include, but are not limited to, ear diseases, autoimmune ear diseases (Yoo T J. et al., Cell Immunol 1994 August; 157 (1): 249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2): 49) and systemic sclerosis (Renaudineau Y. et al., Clin Diagn Lab Immunol. 1999 March; 6 (2): 156); Chan O T. et al., Immunol Rev 1999 June; 169:107) and Familial Mediterranean fever (FMF).

### Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

According to a specific embodiment, the inflammatory disease is an infectious disease.

According to a specific embodiment, the inflammatory disease is a viral disease.

Viral associate diseases include, but are not limited to, diseases caused by corona viruses, including Sars-COV 2/Covid-19, HIV, EBV, varcerella viruses and associated conditions such as shingles, Hep C and Hep B virues.

Fungal diseases include, but are not limited to, aspergillosis and chronic lung diease resulting from aspergillosis, Candida auris infections.

According to a specific embodiment, the inflammatory disease is a Coronavirus infection (e.g., COVID19).

### Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

### Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy, poison oak, and poison sumac allergy and food allergies, such as milk allergy, tree or peanut allergies, wheat/gluten allergies, egg allergy etc.

Further examples of diseases with an inflammatory component include cholesterol disorders, hair loss, depression, hormonal disorders.

### Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a specific embodiment, the cancer is a non-solid tumor, e.g., blood/hematologic cancer.

According to a specific embodiment, the inflammatory disease is not a solid tumor.

### Skin Conditions

Skin conditions associated with skin aging and inflammation include, but are limited to, wound repair and wound scarring, age spots, freckles and other uneven pigmentation of the skin, lose of elasticity in the skin from aging, acne, body odor, eczema, proriasis, and rashes or hives and premature and/or general graying of hair, and scalp issues (e.g., dandruff, seborrheic dermatitis and hair loss) . . .

### Neuropsychological Conditions

Numerous neuropsychological condition have been shown to have a link with chronic inflammation, including but not limited to, anxiety, depression, loss of focus, ADHD, ADD, PTSD, deliurm, irregular sleep pattern including, sleep apnea, insomnia, manic depression, schizophrenia, borderline personality disorder, OCD, Aspergers, eating disorders, addiction, autism, brain fog, verbal memory deficit, vascular diseases of the brain etc; Parkinson's disease and associated symptoms including tremors and freezing gait; Alzheimer's disease symptoms including memory loss, anxiety, motor and psychological function; panic disorder, disorientation, restless leg syndrome, tremors and twitches; tics associated with Tuerettes, irregular speech, stutters etc

Also contemplated with the invention is the alleviation of secondary symptoms associated with mental retardation, Down Syndrome individuals, including but not limited to the early onset of Alzheimer's and problems associated with abnormal growth hormone levels.

Also contemplated are other neurologically related diseases and conditions related to vision loss, e.g. macular degeneration, cataract, diabetic retinopathy, and glaucoma.

### Hormonal and Metabolic-Related Conditions or Diseases

Various hormonal-related conditions or diseases are connected with chronic inflammation including, but not limited to, diseases associated with sex hormones such as, osteoporosis, osteopenia hyperthyroidism and symptoms associated with perimenopause and/or menopause, such as mood changes, anxiety, depression, low self-esteemm, brain fog, hot flashes, joint pain, irregular sleep, insomnia, night sweats, heart palpitations/flutters, headaches and migraines, weight gain, skin changes, including itchy and dry skin, reduced sex drive, vaginal dryness, pain itchig and discomfort during sex, recurrent UTIs and endometriosis; diseases associated with growth hormones, such as gigantism, acromegaly, and pituitary dwarfism; diseases associated with thyroid hormones, such as hyperthyroidism, Grave's disease, Toxic nodular Goiter, Exophthalmos, Hypothyroidism, Myxodema, Congenital hypothyroidism/Cretinism, and autoimmune thyroiditis; diseases associated with adrenal hormones, such as Mineralocorticoid Deficiency, Glucocorticoid Deficiency, and Addison's disease, Cushing's Syndrome, disorders associated with parathyroid hormone, including hyposparathyroidism. Also contemplated with the invention is the treatment of law testosterone or low estrogen.

Assoicated with improved metabolic efficiency of the body is weight loss and an ability of reduce fat storage, thereby treating obsesity. Associated with treating obesity and weight is a reduction of the appearance of cellulite. Also associated with metabolic diseases are hypoxis; uremia, renal failure and encephalitis and tuberculous memomgotos.

In addition, to hormonal disorders affecting adults contemplated as an embodiment of the invention is treating diseases and conditions associated with growth hormones, e.g. an improper level of insulin-like growth factor 1 (IGF-1), insulin-like growth factor binding protein 3 (IGFBP3), somatotropin; pituitary growth hormone), placental growth hormone or growth hormone variant.

### Cholesterol Reduction/Heart Disease/Stroke

Lowering of cholesterol is a key component for reducing the occurrence of heart disease and stroke and ischaemia. The compositions of the invention have an ability to reduce cholesterol levels in patients. Lowering cholesterol has also been connected with increase ability to fight cancer.

Also associated with heart disease and stroke is high blood pressure, which may be reduced with the compositions of the invention. The compostions of the invention may also be used to reduce the risk of or treat heart disease, stroke ischaemia, hypoxia, myocarditis

### Renal Diseases and Disorders

The compositions of the inventions may be useful in preventing or treating conditions associated with diseases and conditions of the kidneys such as, uraemia, interstitial cystitis, and kidney stones.

### Biomarker Modulation/Regulation

The compositions of the invention have been shown to modulate biomarkers, which are involved in inflammatory diseases and other diseases. For example, the compositons of the invention may be used to modulate levels of cholesterol, blood glucose, albumin, alkaline phosphatase calcium, iron, potassium and vitamin D in the blood, C-reactive protein RBC Hematocrit; Haemoglobin and/or platelets.

Also included as possible embodiments of the invention are the treatment of glaucoma and fibrotic formation/function in the cornea,

The composition of matter comprising the component(s) (a plant species or genus thereof-derived component selected from the group consisting of a plant part, extract thereof, fraction thereof, active ingredient thereof, synthetic analog thereof, mimetic thereof or combination thereof, wherein said component is capable of treating inflammation) of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition of matter comprising the components accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intrapulmonary or intraocular injections.

For some embodiments the invention may be administered as a topical composition, e.g. for the treatment of skin and scalp conditions.

In various exemplary embodiments of the invention, the composition is provided as a pharmaceutical or dietary supplement dosage form suitable for oral administration. Dosage forms suitable for oral administration include tablets, soft capsules, hard capsules, pills, granules, powders, emulsions, suspensions, sprays, syrups and pellets. In various other embodiments of the invention, the composition is provided as a pharmaceutical dosage form suitable for parenteral administration such as liquid formulations for administration as drops or by injection, or as solid or semisolid dosage forms for suppositories. The composition of the invention may also be delivered in the form of a slow release depot compostion, which may be administered by injection e.g. subcutaneously or intramuscularly, allowing for dosing every month, once every 2 months, once every 3 months etc.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport polypeptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin polypeptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

In specific embodiments, the components and/or compositions of the invention are provided in a form suitable for administration by inhalation or nasal administration.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Compositions for depot administration may be in the form of a polymer matrix or micro or nano particles.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (composition of matter comprising the components accountable for the biological effect) effective to prevent, alleviate or ameliorate symptoms or progress of a disorder (e.g. inflammatory disease) or prolong the survival of the subject being treated.

Determination of a therapeutically effective amount is well within the capability of those skilled in the art, especially in light of the detailed disclosure provided herein.

For example, any in vivo or in vitro method of evaluating the severity of the inflammation or related symptoms may be employed.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans. Description of some relevant animal models for inflammatory diseases and autoimmune diseases are provided infra.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. 30 (See e.g., Fingl, et al., 1975, in “The Pharmacological Basis of Therapeutics”, Ch. 1 p.1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In another embodiment, the invention provides a nutritional or dietary compositions in the form of foods or beverages, which comprise the component(s) described herein. These foods or beverages comprise various exemplary embodiments of the inventive compositions. These foods or beverages can be prepared or provided as cereals, baby foods, healthy foods, or food for specified health uses such as solid food like chocolate or nutritional bars, semisolid food like cream or jam, or gel; and also as beverages. Specific and non-limiting examples of such food or beverage items include refreshing beverages, lactic acid bacteria beverages, drops, candies, chewing gum, chocolate, gummy candy, yoghurts, ice creams, puddings, soft adzuki bean jellies, jellies, cookies and the like.

The plant-derived component or components of the present invention can be administered with other medications to increase therapeutic bioavailability, boost therapeutic efficacy, and minimize side effects.

Anti inflammatory drugs that can be administered in combination with the compositions of some embodiments of the invention include NSAIDS and steroids such as corticosteroids. Examples of anti-inflammatory drugs include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; llonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Mefenamic Dibutyrate; Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

### Systemic Lupus Erythematosus (SLE)

The New Zealand Black (NZB) and the New Zealand White (NZW) mouse, (NZB×NZW) F1, is a spontaneous model which develops a lupus-like disease.

### Multiple Sclerosis

For example, experimental autoimmune encephalomyelitis (EAE) has been widely used as a model of multiple sclerosis. In this model, spinal cord homogenate or a protein derivative such as myelin basic protein is injected with a mixture of potent immunostimulants, most commonly in mice from the SJL strain.

### Diabetes

Five animal models of spontaneous diabetes are mainly preferred for studying autoimmune diabetes: the NOD mouse, the diabetes-prone BB rat, the LETL rat, the KDP rat and the LEW-iddm rat. NOD mouse and BB rat are the most widely used.

### Rheumatoid Arthritis

Animal models have been used extensively in studies of rheumatoid arthritis pathogenesis. Despite the inherent limitations of all animal models, several rodent models have significantly progressed our understanding of the fundamental mechanisms underpinning rheumatoid arthritis and contributed to several current major advances in treatment. These models include the induced arthritis models such as collagen-induced arthritis, collagen-antibody-induced arthritis, zymosan-induced arthritis, and the methylated BSA model, and the genetically manipulated or spontaneous arthritis models such as the TNF-alpha-transgenic mouse, K/BxN mouse, and the Skg mouse.

### Crohn's Disease (CD) and Ulcerative Cholitis (UC)

### Dextran Sulfate Sodium

Dextran sulfate sodium (DSS) is a polyanionic derivative of dextran with a chemical formula of (C₆H₇ Na₃O₁₄S₃)n. DSS is most commonly administered in the drinking water for peroral treatment of the animals with the compound. The concentration of the compound which is often used is 3%. An inflammatory response is initiated by DSS in wild-type animals which starts distally after about 5 days and is limited to the colonic mucosa. It is still not well understood how DSS starts the inflammation in the colon. However, a recent study investigating DSS both in vitro and in vivo revealed that DSS has a direct effect on the inner mucus layer, leading to bacterial penetration of this layer before any inflammatory signs could be seen. Thus, it can be concluded that a loss of the inner colon mucus layer is the initial episode leading to bacterial penetration and ultimately, the development of an inflammatory response.

### 2,4,6-Trinitrobenzene Sulfonic Acid

Trinitrobenzene sulfonic acid (TNBS) is an oxidizing Nitroaryl compound which is administered intrarectally in animals to induce IBD. It causes induction of colonic damage which leads to necrotic regions associated with inflammatory areas. High myeloperoxidase activity causes damage mainly characterized by neutrophilic infiltration into the colonic tissue. An increase in the mucosal permeability is a result of the damage to the colonic epithelium and interstitium. TNBS may cause a decrease in the mucosal hydrophobicity by interacting with the phospholipids present on the surface of the colonic mucosa. This decreased hydrophobicity is believed to contribute to TNBS-induced inflammation of the colon. TNBS causes necrosis and deep tissue damage which mimics the transmural involvement of CD; hence, it may be preferred to be a better experimental model of CD rather than UC.

TNBS-induced colitis models have helped to be an important source for generating vital information about the cytokines involved in the human IBD. It has also helped in shaping the therapy regimens of the human disease.

### Oxazolone Colitis

Intrarectal administration of the hapten compound oxazolone along with ethanol in animals causes acute colitis. Oxazolone leads to acute superficial mucosal inflammation in the distal part of colon. There is colonic infiltration by lymphocytes and neutrophils along with associated edema in lamina propria. There is type helper 2 (Th2) cell-mediated immune response with an elevation in the production of interleukins. This animal model is distinguished from TNBS-induced colitis by the presence of Th2-mediated response instead of Th1 found in the TNBS model.

### Acetic Acid-Induced Colitis

Administration of diluted acetic acid through the rectal route is another method to induce colitis in rodents. The treatment with acetic acid causes colonic mucosal damage which leads to a condition similar to UC. [10] MacPherson and Pfeiffer were the first ones to demonstrate this model where they administered 10%-50% acetic acid intrarectally to the rat for 10 s, followed by flushing the lumen three times with saline. Acetic acid caused diffuse colitis in a dose-dependent manner in these rodents, with histopathological features including ulceration of the distal colon and crypt abnormalities. The latest practice utilizing 4% acetic acid for 15-30 s. The low cost of the chemical as well as the ease of administration are few advantages of acetic acid-induced colitis model. The epithelial injury induced by acetic acid is not immunological in the first 24 h. Thus, drugs which target the immune responses should be evaluated after 24 h of induction.

### Salmonella-Induced Colitis

Salmonella typhimurium and Salmonella Dublin are Gram-negative bacteria that can cause foodborne intestinal diseases. Direct administration of S. typhimurium to mice orally causes a systemic infection that may resemble the picture of intestinal inflammation after pretreatment with oral antibiotics. The pretreatment helps to disturb the normal bacterial microflora causing high growth of S. typhimurium within 1 day. The intestinal inflammation caused by such colonization has histopathological characteristics which are similar to the human UC in terms of epithelial crypt damage and infiltration of neutrophils. The induction of colitis causes the systemic infection within 5-7 days of infection.

Adherent-Invasive Escherichia coli

Adherent-invasive Escherichia coli (AIEC) could adhere to the epithelial cells of both small and large intestine with equal affinity. However, AIEC infection cannot induce colitis on its own. During the entire course of AIEC infection, colonic inflammation is induced in animal models using the infection along with low-dose DSS administration to cause mild epithelial damage. Disruption of the intestinal microflora, including the probiotic biofilm, is caused by certain antibiotics which lead to the development of an ideal environment for the opportunistic AIEC to adhere to and invade IECs and macrophages. The changes induced by this model closely resemble the human UC.

### Adoptive Transfer Models of Colitis

The adoptive transfer model includes the process of transferring T-cells or immune tissue from one mouse into an adoptive host leading to the development of colitis. The various donors and hosts which have been used include:
CD4+ T-cells transferred into severe combined immunodeficiency (SCID) mice;
hsp60-specific CD8+ T-lymphocytes into T-cell receptor−/− or SCID mice;
CD4+CD25-T-cells into SCID mice.

The adoptive models are well-characterized models of chronic colitis induced by disturbing the T-cell homeostasis. These models are particularly useful in understanding how different T-cell populations might contribute to the pathogenesis of IBD as they rely on the transfer of T cells.

### Genetic Models of Colitis

The advancement in the genetic technologies has resulted in the development of multiple genes whose variants may be related to elevated predisposition to IBD. Tools such as genome-wide association study have recognized susceptibility genes. The various murine models containing relevant genetic variants, or those incorporating these newly identified variants, have been used to further explore the genetic contribution to colitis.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein the term “viral entry mechanism” refers to viral proteins that mediate entry into cells. The viral entry mechanism proteins include attachment proteins and other proteins that are required for entry of non-enveloped and enveloped viruses into cells. Different viruses use different entry proteins, however, both non-enveloped and enveloped viruses share the same two main steps and routes of virus entry; (1) attachment to cell-surface receptors (2) conformational changes of the viral entry proteins or the host-cell receptors, the viral entry can occur either by penetration of the cell membrane (for non-enveloped viruses) or fusion (for enveloped viruses) to the cell membrane (see “Virus entry: molecular mechanisms and biomedical applications”, Dimitrov, 2004)

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Case Report

A 34 years old pregnant woman diagnosed with gestational diabetes and treated with glutamin was administered daily with 5 ml sesame oil 100% w/v (commencing on Sep. 23, 2020 for 4 days) and then co-administered with 5 ml sesame oil 100% w/v (Naissance) and 5 ml 100% w/v Nigella sativa oil (Better Flex). During oil treatment glucamin was not administered.

Blood glucose levels (mg/dL) were determined at the indicated hours.

Results are shown below.

| 6 AM | 11 AM | 15 PM | 21 PM | Date |
|---|---|---|---|---|
| 172 | 154 | 122 | 97 | 10 Sep. 2020 |
| 125 | 109 | 126 | 103 | 11 Sep. 2020 |
| 101 | 88 | 106 | 95 | 12 Sep. 2020 |
| 113 | 84 | 125 | 80 | 13 Sep. 2020 |
| 94 | 90 | 134 | 125 | 14 Sep. 2020 |
| 158 | 159 | 98 | 83 | 15 Sep. 2020 |
| 148 | ND | 154 | 88 | 16 Sep. 2020 |
| 99 | 124 | 90 | 77 | 17 Sep. 2020 |
| 94 | 84 | 89 | 84 | 18 Sep. 2020 |
| ND | ND | 109 | 74 | 21 Sep. 2020 |
| 151 | 86 | 73 | 81 | 23 Sep. 2020 |
| 166 | 154 | 89 | 85 | 24 Sep. 2020 |
| 112 | 134 | 99 | 80 | 25 Sep. 2020 |
| 126 | 112 | 94 | 90 | 26 Sep. 2020 |
| ND | ND | 70 | 83 | 27 Sep. 2020 |
| 91 | 136 | 74 | 80 | 28 Sep. 2020 |
| 130 | 83 | 113 | 85 | 29 Sep. 2020 |
| 116 | 92 | 104 | 82 | 30 Sep. 2020 |
| 101 | 96 | 96 | 95 | 1 Oct. 2020 |
| ND | ND | 151 | 78 | 2 Oct. 2020 |

ND = Not determined

### Example 2

### a) Reduction of Blood Glucose in a Canine Subject

Tests were carried out on a canine subject before and after oral dosage of the composition containing Complex D of the present invention.
Animal details: Simba, dog, female
History/Physical examination/Other findings:
Type of test: Blood count and biochemistry

The treatment reduced blood glucose from 123 mg/dL to 64 mg/dL measured 2 weeks after beginning treatment.

| | BEFORE | AFTER |
|---|---|---|
| Blood Glucose | 123 mg/dL | 64 mg/dL |
| NEU# | 3.17 | 5.47 |
| MONO | 2.6 | 5.0 |
| MONO# | 0.15 | 0.45 |

### b) Reduction of Blood Glucose in Human Patient

A patient with diabetes Type I who is on insulin was given the composition of the invention. The patient's average blood glucose prior to treatment with the composition of the invention was 182 mg/dL. After taking the composition of the invention significantly reduced to 117 mg/dL and a day later to 53 mg/dL.

### Example 3: Reversal of Alopecia and Weight Loss in a Human Subject

A woman was treated with mixtures of the present invention over a period of 2-3 months resulting in the reversal of hair loss and weight loss of 4 kg.

### Example 4

Assays for destroying viral entry mechanism proteins by plant components.

COVID-19 serves as a model for viral entry mechanism attenuation and modification. COVID-19 is caused by severe acute respiratory syndrome coronavirus 2 (SARS-COV-2). The cell membrane ACE-2 receptor is an attachment and entry site for SARS-COV-2. The ACE-2 receptor is a type I transmembrane metallocarboxypeptidase with homology to ACE, an enzyme long-known to be a key player in the Renin-Angiotensin system (RAS) and a target for the treatment of hypertension. There is evidence that SARS-COV-2 utilizes ACE-2 as a cellular entry receptor. Zhou et al. showed that SARS-COV-2 could use ACE-2 from humans, Chinese horseshoe bats, civet cats, and pigs to gain entry into ACE-2-expressing Hela cells (See Zhou, P., Yang, X L., Wang, X G. et al. A pneumonia outbreak associated with a new coronavirus of probable bat origin. Nature 579, 270-273 (2020)). The spike(S) protein of SARS-COV-2, which plays a key role in the receptor recognition and cell membrane fusion process, is composed of two subunits, S1 (120 kDa) and S2 (80 kDa). The S1 subunit contains a receptor-binding domain that recognizes and binds to the host receptor angiotensin-converting enzyme 2 (ACE-2). The S2 subunit mediates viral cell membrane fusion by forming a six-helical bundle via the two-heptad repeat domain (see Huang, Y., Yang, C., Xu, Xf. et al. Structural and functional properties of SARS-COV-2 spike protein: potential antivirus drug development for COVID-19. Acta Pharmacol Sin 41, 1141-1149 (2020)). Interfering, attenuating, impairing the function of the S1 and S2 subunits will eventually lead to an attenuated, impaired and less infective virus.

It is noted that in addition to is role in viral infectivity ACE2 may also play a role in inflammation generally. ACE2 generates Ang 1-7, which serves a critical role in counterbalancing the vasoconstrictive, pro-inflammatory, and pro-coagulant effects of ACE-induced Ang II.

### Viral Protein Digestion Assays

The materials used in all the following viral protein digestion assays are disclosed in table 1.

TABLE 1

| | Company | cat # |
|---|---|---|
| Recombinant SARS-CoV-2 S1 Subunit | RayBiotech | 230-30161 |
| Recombinant SARS-CoV-2 S2 Subunit | RayBiotech | 230-30163 |
| Recombinant SARS-CoV-2 Nucleocapsid | RayBiotech | 230-30164 |
| Proteinase K | Sigma | P4850 |
| Instant Blue | Expedeon | I581L |
| DMSO | SIGMA | D2650 |
| Criterion ™ TGX Stain-Free Precast | BIORAD | 5671084 |
| Midi Gels 4-15%, 18 wells, 30 μl | | |
| 10X Tris/Glycine/SDS | BIORAD | 161-0772 |
| 4X Laemmil Sample Buffer | BIORAD | 1610747 |
| 8-Mercaptoethanol 14.2M | SIGMA | M3148 |
| Precision protein standard | BIORAD | 161-0373 |

Each of the tested plant based treatment was numbered as disclosed in table 2 and the tested combinations complexes were classified as disclosed in table 3.

| Treatment No. | Name |
|---|---|
| 1 | Oregano Oil |
| 2 | Thyme Oil |
| 3 | Nigela Sativa |
| 4 | Sumac Oil |
| 5 | Sesame Oil |
| 6 | Olibanum Oil |
| Complex A | Oils 1 + 2 + 3 |
| Complex B | Oils 1 + 2 + 3 + 4 |
| Complex C | Olls 1 + 2 + 3 + 4 + 5 |
| Complex D | Oils 1 + 2 + 3 + 4 + 5 + 6 |

Oils mixtures were prepared by mixing equal amounts of each oil. The mix was then diluted 1:2 with DMSO, to acquire a solution of 50% DMSO, 50% Oil mix and the final reaction concentration was 5% oil mix, 5% DMSO.

For each assay reaction, 1 μg protein per reaction was incubated with 3 μl of the oil mixture at final reaction volume of 30 μl. The reaction was incubated for 6 hours at 37° C.

Following incubation, the reaction was stopped by adding 10 μl/reaction of sample buffer 4X and incubation 10 minutes at 72° C. Samples were then run in 4-15% TGX Criterion Gel (BIORAD) for 50 minutes at 200 Volt. Following run, the gel was incubated for 1 hour with Instant Blue reagent (Expedeon) and further washed with water until distinct bands were observed.

Densitometry was preformed, pictures were analyzed with ImageJ software.

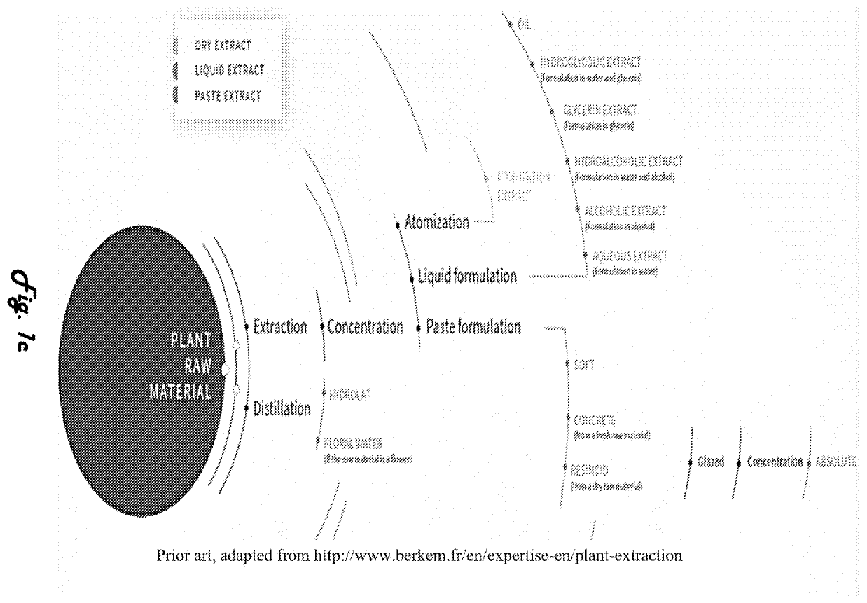

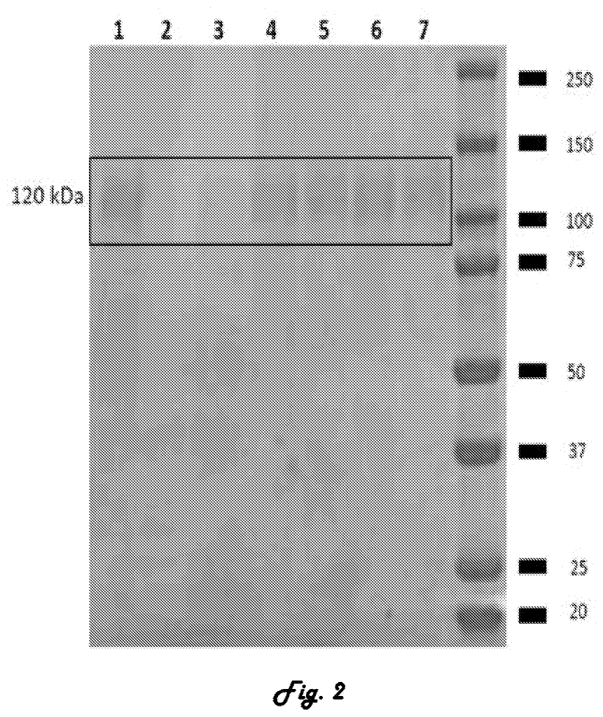

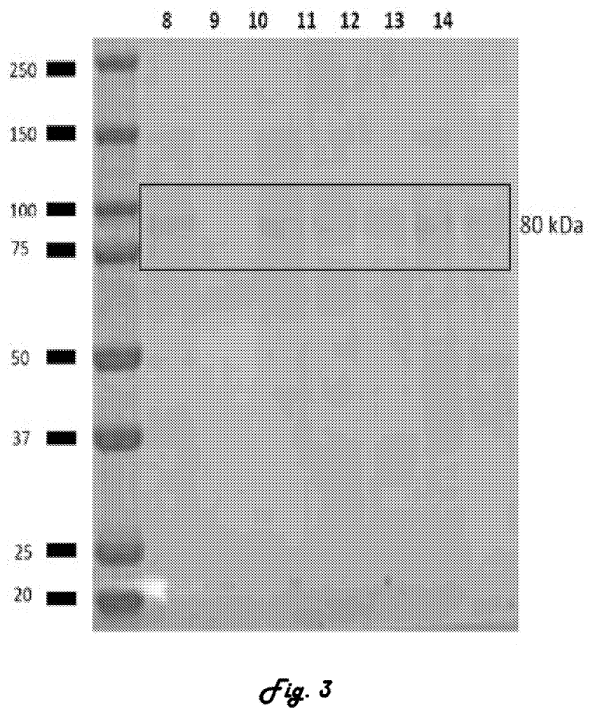

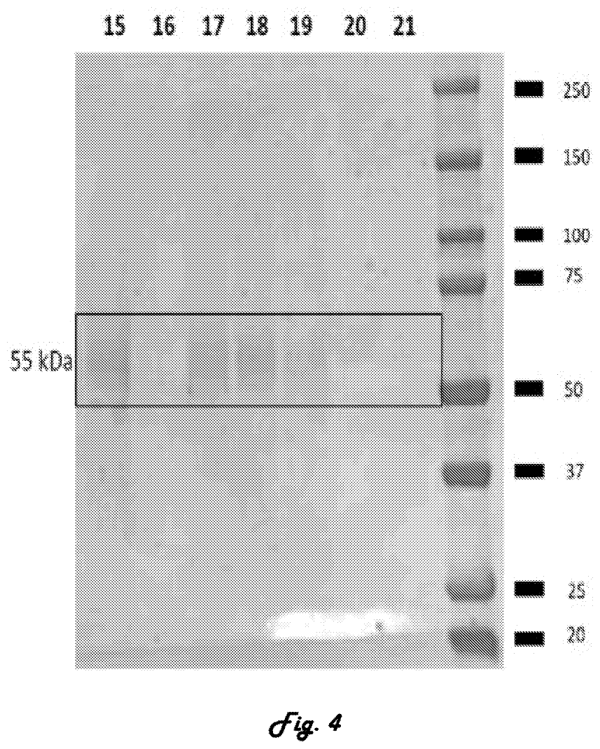

The protein digestion assay was conducted as disclosed in table 4. SARS-COV-2 S1 subunit, SARS-COV-2 S2 subunit, SARS-COV-2 Nucleocapsid protein and a negative control with no protein, were incubated with different plant oils or combinations for 6 h at 37° C. and subsequently run on SDS-page, stained with “instant blue” for the presence of proteins in the gel. The untreated control appeared at the expected molecular weight and the effect of different treatments was compared to this control (see FIGS. 2-4). Significant disappearance of the protein was observed following treatment with the protease proteinase K.

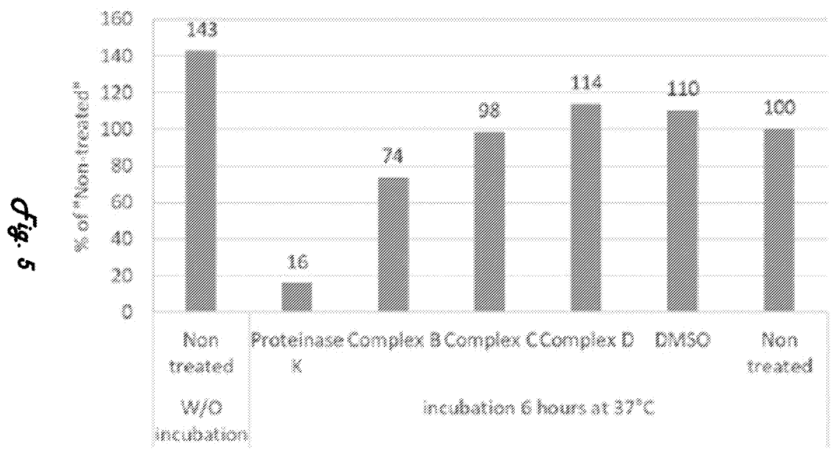

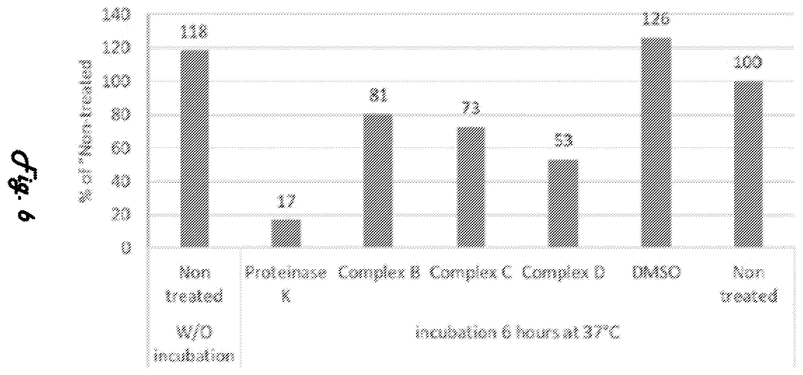

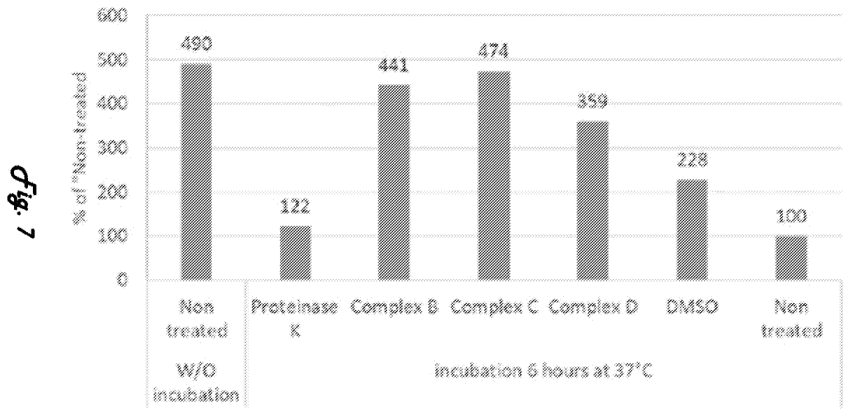

Densitometry of the SARS-COV-2 S1 subunit, SARS-COV-2 S2 subunit, SARS-COV-2 and the Nucleocapsid protein assays disclose that although the Nucleocapsid protein underwent little to no digestion with either of the tested treatments as compared to the protein K treatment the two SARS-COV-2 subunits S1 and S2 underwent a substantial digestion (see FIGS. 5-7 respectively).

To conclude, the viral protein digestion assay demonstrates that there is a significant digestion of the S1 and S2 subunits without destroying the Nucleocapsid protein.

### Recombinant SARS-Cov-2 S1 Subunit

Following incubation of the protein with a mix prepared from equal volumes from items 1+2+3+4 (complex B), a 26% reduction in the protein signal was observed.

### Recombinant SARS-Cov-2 S2 Subunit Protein

Following incubation of the protein with a mix prepared from equal volumes from items 1+2+3+4 (complex B), a 19% reduction in the protein signal was observed. Following incubation of the protein with a mix prepared from equal volumes from items 1+2+3+4+5 (complex C), a 27% reduction in the protein signal was observed Following incubation of the protein with a mix prepared from equal volumes from items 1+2+3+4+5+6 (complex D), a 47% reduction in the protein signal was observed.

These significant digestion rates of both S1 and S2 subunits of the spike protein are likely to result in the subsequent attenuation of the Coronavirus cell attachment and internalization mechanism. It is clear that attenuating a virus cell attachment and internalization mechanism, entry mechnism, disease levels and viral load can be reduced in an infected subject, or prevent an uninfected subject from getting infected. Subsequently, viral mediated or/and triggered inflammatory and viral mediated or/and triggered Diabetes Mellitus (type 1 or type 2) occurrence may be reduced.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### Example 5—Case Report Weight Loss

A woman was treated with mixtures of the present invention.

Within up to 17 days she has lost 5 kg.

Ronit, a volunteer from Israel with a weight of 80 kg has lost 3 kg in two weeks after taking the composition of the invention.

Inbar, another volunteer from Israel went from 74 kg to 68 kg in 6 weeks after taking the composition of the invention.

### Example 6—case report Dementia

A man was treated with mixtures of the present invention including one or more essential oils comprise one or more of carvacrol, thymol, nigella sativa, sumac, sesame, Boswellia Sacra and combination thereof.

Within the first 3 week of treatment, the patient demonstrated very significant improvement in his cognitive ability and including speech communication—he started communicating after two years of silence, recognition of devices, relaxed mood and smiles, mobility—he started walking after 6 months of sitting, vitality—the color in his face and his eyes returned

### Example 7—Case Report Dementia and Anemia and Increase in Protein and Iron and Blood Results

A woman in her early 80s with Alzheimer's disease for 3 years diagnosed with a “confusion” state, and apathy by a geriatric doctor was treated with mixtures of the present invention.

\Within the first 21 days of treatment, the patient demonstrated very significant improvement in her cognitive ability. Prior to receiving the treatment she was in her own world, did not talk much and had no appetite. Within the first 3 weeks she started talking more, greeting her neighbors with good evening and good morning, commenting on her daughter's appearance, asking her to contact her more often, smiling more, asking questions, being more interested in activities in her care home and for the first time in 3 years-prepared her own coffee. Her breathing got better and she also was able to stand longer without the aid of a chair in the shower.

In addition changes in her blood results include (first number is before the treatment, second number is after her treatment): WBC: 7.2→8 | RBC: 3.82→4.17 | Hemoglobin: 10.9→11.6 | Hematocrit: 34.7 →37.2| Protein 5.8→6.3 | Fe-Iron: 39→53 |

### Example 8—Case Report Dementia

A woman diagnosed with Alzheimer's disease was treated with a composition of the invention.

Prior to receiving the treatment, the patient did not speak rationally at all. Within the first 21 days of treatment the woman became more aware of her environment. E.g when her son took her photo she asked “Am I being photographed?” or “Why are you upset” She also started sleeping much better than before the start of the treatment.

### Example 9

A woman by the name of Kinnert was treated with a composition of the invention. The following symptoms have significantly improved—the numbers reflect her subjective feeling from 0 to 10 whereas 10 is the most severe: the numbers reflect the day before the treatment, first day of treatment, second day, third day respectively.

Headaches: 10,7,4,4 | Throat aches: 8,5,2,2 | Diarrhea: 2,0,0,0 | Nausea: 10,0,0,1

Vomiting: 10,0,0,0 | Muscles aches: 10,8,3,4 | Fatigue: 10,7,5 | Loss of smell and taste: 6,3,1,1 | Runny Nose: 4,3,2,1 |hearing problems: 8,6,6,5 |short memory loss problem: 8,6,6,5

### Example 10

A man by the name of Gilad who was tested Positive to Covid was treated with mixtures of the present invention.

After 24 hours from the start of the treatment-his Covid 19 test was negative.

The following symptoms have significantly improved—the numbers reflect her subjective feeling from 0 to 10 whereas 10 is the most severe: the numbers reflect the day before the treatment, first day of treatment, second day, third day respectively.
Headaches: 8,7,6,6
Diarrhea: 6,0,0,0
Muscle aches: 8,7,5,5
Fatigue and tiredness: 8,7,6,5
Loss memory: 7,5,5,5
Dizziness: 8,5,5,5
Focus: 7.5,5,5

### Example 11

A man by the name of Uri who was tested Positive to Covid was treated with mixtures of the present invention.

After 24 hours from the start of the treatment—his Covid 19 test was negative. The following symptoms have significantly improved—the numbers reflect her subjective feeling from 0 to 10 whereas 10 is the most severe: the numbers reflect the day before the treatment, first day of treatment, second day, third day respectively
Fever: 37.8, 36.5, 36.5
Heavy coughing 8,5,4
Headaches 5,0,0
Throat aches 5,0,0
Muscles aches 9,0,0
Fatigue and Tirdeness: 7,3,2
Loss of taste and smell: 10,4,2
Loss of Sleep 2,0

### Example 12—Reduction of Irregular Skin Pigmentation/Freckles

An Israeli some showe marked reduction of sun and age-induced face freckling after application of a composition of the invention.

### Example 13—Mango and Sleep

A patient suffering from chronic insomnia was given a mixture of a composition of the invention in combination with a mango extract. The patient reported sleeping for six hours after taking a composition of the invention.

### Example 14—Modulation of Blood Iron Levels, Glucose, Vitamin D and/or C-Reactive Protein

Patient 1 was a composition of the invention.

| | Jul. 5, 2022 | Jul. 20, 2022 | Aug. 8, 2022 |
|---|---|---|---|
| Glucose mmol/L | 6.1 | 5.6 | 6.0 |
| C-reactive | 10.8 | 4.3 | 5.0 |
| protein mg/L) | | | |
| Haemoglobin (g/L) | 125 | 142 | 140 |
| Iron | | | |
| Ca (mmol/L) | 2.29 | 2.32 | 2.30 |
| Potassium (mmol/L) | 4.3 | 4.5 | 4.5 |
| Creatine (umol/L) | 47 | 54 | 58 |
| Cholesterol (mmol/L) | 4.4 | 4.8 | 4.0 |

Patient 2 was given a composition of the invention.

| | Jul. 6, 2021 | Aug. 8, 2021 |
|---|---|---|
| RBC (M/μl) | 3.82 | 4.17 |
| Hematocrit (g/dl) | 34.7 | 37.2 |
| Haemoglobin % | 10 | 11.6 |
| Iron (Ug/dL) | 39 | 53 |

Patient 3 was a composition of the invention.

| | Dec. 16, 2021 | Jan. 6, 2022 |
|---|---|---|
| Glucose (mg/dL) | 100 | 81 |
| Cholesterol (mg/dL) | 256.6 | 244.9 |

Patient 4 was given a composition of the invention.

| | Sep. 9, 2021 | Oct. 11, 2021 |
|---|---|---|
| C-reactive | 1.66 | 0.07 |
| protein (mg/dL) | | |
| Glucose (mg/dL) | 103 | 87 |
| Vitamin D (nmol/L) | 82.2 | 138.0 |
| RBC (M/μl) | 4.47 | 4.38 |
| Hematocrit % | 39.6 | 37.7 |
| Haemoglobin (g/dl) | 13.5 | 12.9 |
| Cholesterol (mg/dL) | 114.6 | 99.3 |

Patient 5 was given a composition of the invention . . .

| | Dec. 17, 2021 | Jan. 6, 2022 |
|---|---|---|
| Glucose (mg/dL) | 100 | 81 |
| Cholesterol (mg/dL) | 256.6 | 244.9 |

Patient 6 was given a composition of the invention.

| | May 23, 2022 | Jul. 21, 2022 | Aug. 15, 2022 |
|---|---|---|---|
| C-reactive | 42.27 | | 0.09 |
| protein (mg/dL) | | | |
| Glucose (mg/dL) | 102 | 97 | 94 |

Patient 7, who has chronic lung disease was given the composition of the invention.

| | 16 Dec. 2021 | 6 Jan. 2022 |
|---|---|---|
| Glucose | 100 | 81 |
| Urea | 30.9 | 25.2 |
| Creatinine | 1.05 | 0.84 |
| Sodium | 141.6 | 140.2 |
| Potassium | 5.2 | 4.4 |
| Calcium | 9.12 | 8.49 |
| Phosphorus | 2.15 | 2.04 |
| Protein Total | | 6.86 |
| Albumin | | 3.99 |
| Cholesterol | 254 | 244 |
| TriGlyCerides | 185 | |
| Cholesterol HDL | | 60 |
| Cholesterol LDL | | 148 |
| AlkPhospatase | 70 | 54 |

Patient 8—with three years dementia

| | First test | Second test | Normal |
|---|---|---|---|
| | 15 Jul. 2021 | 5 Sep. 2021 | Values |
| Protein total | 5.8 | 6.3 | 6.6-8.3 |
| Fe-Iron | 39 | 53 | 60-180 |
| RBC-Red Blood Check | 3.82 | 4.17 | 4-5.2 |
| Hemoglobin | 10.9 | 11.6 | 12.5-16 |
| WBC | 7.2 | 8 | |
| Hematocrit | 34.7 | 37.2 | 36-37.2 |

1.-49. (canceled)

50. A method of treating an inflammatory disease or condition associated with inflammation in a subject in need thereof, the method comprising administering to the subject an effective amount of a pharmaceutical composition or food supplement comprising extracts of Nigella sativa, Thymus, Origanum and Rhus coriaria.

51. The method of claim 50, wherein said pharmaceutical composition or food supplement comprises at least one of thymoquinone or an analog thereof, thymol or an analog thereof, carvacrol or an analog thereof, bromelain or an analog thereof, tryptophan or an analog thereof.

52. The method of claim 50, wherein said inflammatory disease comprises an autoimmune disease.

53. The method of claim 50, wherein said inflammatory disease comprises an acute inflammatory disease.

54. The method of claim 50, wherein said inflammatory disease comprises Type I, Type II or gestational diabetes.

55. The method of claim 50, wherein said inflammatory disease is selected from Hashimoto's disease, Grave's disease, Goiter disease, Thyroid Nodules disease, irritable bowel syndrome, Crohn's disease, colitis, gastritis, dyspepsia, of allergies, candidiasis, cold related disorders, cholesterol disorders, hair loss, depression, hormonal disorders, obesity and any combination thereof.

56. The method of claim 50 wherein the pharmaceutical composition or food supplement further comprises “Beduin Tea” comprising Rose Leaves Micromeria fruticose, Salvia, cymbopgon (Citral,) Aloysia, verbena officinalis, Origanum majorana, and/or menthe.

57. The method of claim 50 wherein the pharmaceutical composition or food supplement further comprises “Beduin Tea” comprising sage, cardamom, cinnamon, black tea, habuk, and/or Marmaya.

58. The method of claim 50, wherein pharmaceutical composition or food supplement further comprises at least one of Sesamum indicum, Gynostemma pentaphyllum, Boswellia sacra and Panax ginseng.

59. A method for treating gestational diabetes and lowering blood glucose levels in a patient, comprising administering 5 ml daily of Sesame oil of 100% w/v to said patient for first prescribed period of time; and co-administering 5 ml daily of said Sesame oil 100% w/v and 5 ml daily of Nigella Sativa oil of 100% w/v for second prescribed period of time.

60. The method of claim 59, wherein said first prescribed period of time is four days, and said second prescribed period of time is 18 days.

61. A pharmaceutical treatment regimen for reversing alopecia and/or weight loss in a patient, said regimen comprising administering to said patient 2-15, per day of a pharmaceutical composition or food supplement comprising extracts of Nigella sativa, Thymus, Origamim and Rhus coriaria for a prescribed period of between two and three months.

62. The pharmaceutical treatment regimen according to claim 61, wherein said extract mixtures further comprise compounds selected from thymoquinone, thymol, carvacrol, bromelain and tryptophan or plant extract containing tryptophan and analogs thereof.

63. The method according to claim 50, wherein said inflammation is triggered by said viral infection.

64. A pharmaceutical composition or food supplement comprising extracts of Nigella sativa, Thymus, Origanum and Rhus.

65. A method of reducing cholesterol which comprises administering to a patient in need thereof the pharmaceutical composition or food supplement of claim 64.

66. A method of reducing blood glucose levels which comprises administering to a patient in need thereof the pharmaceutical composition or food supplement of claim 64.

67. A method of modulating at least one of albumin, alkaline phosphatase calcium, iron, potassium and vitamin D in the blood which comprises administering to a patient in need thereof the pharmaceutical composition or food supplement of claim 64.

68. A method of modulating C-reactive protein which comprises administering to a patient in need thereof the pharmaceutical composition or food supplement of claim 64.

69. A method of modulating at least one of RBC Hematocrit; Hemoglobin and platelets which comprises administering to a patient in need thereof the pharmaceutical composition or food supplement of claim 64.

70. The pharmaceutical composition of claim 64 further comprising at least one of mango plants or fruits; ginseng, pineapple fruit extracts, tryptophan, silybin, milk thistle extract, chamomile, anise, star anise, α-bisabol, linalool, an extract of lavender, an extract of bergamot, an extract of rosewood, an extract of basil, an extract of neroli oil, turmeric or extract thereof, mango ginger or an extract thereof and curcumin.
